(19) United States
(12) Patent Application Publication
(10) Pub. No.: US 2024/0409596 A1
(43) Pub. Date: Dec. 12, 2024

(54) NOVEL IMMUNE REGULATOR

(71) Applicant: Virothera Limited, Cambridge, Cambridgeshire (GB)

(72) Inventor: Ursula Adele Gompels, London (GB)

(21) Appl. No.: 18/697,913

(22) PCT Filed: Oct. 6, 2022
(86) PCT No.: PCT/EP2022/077849

§ 371 Date: Apr. 2, 2024

(30) Foreign Application Priority Data
Oct. 6, 2021 (LU) 500712

(51) Int. Cl. C07K 14/52; A61K 38/19

(52) U.S. Cl. CPC C07K 14/521; A61K 38/195

(57) ABSTRACT

A chemokine mixture comprising a first chemokine, or an agonist or antagonist variant thereof, which is suitable for binding to CCR4 and a second chemokine, or an agonist or antagonist variant thereof, which is suitable for binding to CCR5 and/or CCR6. The chemokine mixture is suitable for modulating an immune response.

### FIELD OF INVENTION

The present invention relates to chemokine mixtures, polynucleotide mixtures and compositions suitable for modulating an immune response, and corresponding nucleic acid construct mixtures, nucleic acid constructs, host cells, pharmaceutical compositions and kits. The invention also relates to use of the chemokine mixtures, polynucleotide mixtures and compositions for the treatment of diseases and conditions, and methods of treatment using the chemokine mixtures, polynucleotide mixtures and compositions of the invention.

### BACKGROUND

Many diseases and conditions are associated with an excessive, dysregulated or aberrant immune response. Examples of these are autoimmune diseases such as rheumatoid arthritis, inflammatory diseases, infectious diseases which are associated with excessive immune responses, such as antibody or cytokine storms, as in severe Covid or influenza, or in chronic obstructive pulmonary disease, and conditions such as asthma and allergies. Effective treatment and prevention of these diseases and conditions is currently often limited.

Immunotherapy using advanced therapy strategies holds much promise for the treatment of wide areas of pathology, including infectious disease, cancer and autoimmunity, inflammatory diseases and diseases or conditions associated with an aberrant or dysregulated immune system.

An appropriate immune response is central to control of disease, including infectious, inflammatory or neoplastic, as well as efficient immunisation to prevent or treat infections or cancer. For this there is a key balance between conventional T cells, such as those that mediate adaptive immunity, and regulatory T cell (T-regs), which dampen down an induced response and prevent autoimmunity (Mohr, Atif et al. 2019). In adaptive immunity, the therapeutic response recognises damaged or infected cells and removes it through a complex interplay of innate and adaptive immune responses, with antigen presentation to T-cell subsets resulting in specific memory and effector cells stimulated to clear infected or damaged cells. Equally important is the regulation of this response; after disabling and clearing a pathogen-infected cell or cancer cell, the immune response has to be turned off, otherwise there is the potential for pathogenic autoimmune responses, which can be equally lethal.

The focus for immunisation against pathogens or treating cancer has been to increase immune responses, using adjuvants. The focus for treatment of autoimmunity has been more limited, to decrease the immune response, for example using steroids such as dexamethasone for Covid runaway responses. Immunisation of some pathogens has not been successful, for example, persistent infections such as herpesvirus or HIV, which can establish latent infections. Moreover, durability of the response is a concern, for example, the new RNA vaccines for Covid have limited efficiency over time, lasting only a few months for efficient antibody responses and require booster vaccines (Bar-On, Goldberg et al. 2021; Eyre, Taylor et al. 2021). Immunisation against cancer have equally not been successful as the cancer can also evade immune responses or attract regulatory leukocytes. On the other side, the limitation of autoimmune responses, such as runaway responses as in severe Covid, influenza cytokine storms, Crohn's disease, or sepsis, has similarly not provided efficient treatments.

New cancer therapies have turned to strategies for ‘regulating the regulators’ by providing an inhibiting antibody treatment for ‘checkpoints’ of the immune response.

However, these only work for a minority of patients. Nonetheless, a focus on regulation of the response could be equally applicable for new solutions to autoimmunity as well as immunisation. Central to this are the T-reg populations and the chemokine system that can mediate recruitment of regulator leukocytes (Lu, Barbi et al. 2017, Luo and Li 2018, Mohr, Atif et al. 2019). Chemoattracting T-regs can dampen down an aberrant immune response, for example, late stage severe Covid or inflammatory conditions such as arthritis or skin inflammatory conditions (Ikebuchi, Fujimoto et al. 2019, Mohr, Atif et al. 2019). Conversely blocking chemoattraction of T-regs can could keep immune stimulatory conditions favouring the balance of conventional T-cells mediating adaptive immunity, for example in cancer therapy (Ohue and Nishikawa 2019).

Previous studies show that blocking immune-suppressors is a well-defined mechanism for the application of antibody molecules, namely “immune check-point inhibitors” in oncology. They are directed either to the receptor or ligand pair of signaling molecules on immunesuppresive T-lymphocytes which normally add ‘brakes’ to immune signalling, for instance to prevent breaking of immune tolerance and unleashing the damaging self immune responses of an ‘autoimmune’ reaction. This strategy has been used successfully on terminal cancer patients; however it has inherent safety risks and only works on a minority of patients (<30%) (Ikebuchi, Fujimoto et al. 2019), with a success rate that would not favour small molecule development. This strategy also demands additional resources in genetic profiling of patients and their cancers to determine susceptibility to this method via specific receptor expression on the tumours. There is, therefore, a need to develop new methods for applying immunotherapy to more people and with greater success.

Chemokines have been utilised as molecular adjuvants in experimental vaccine formulations and also for development of potential cancer immunotherapy (Mohan, Zhu et al. 2018, Ohue and Nishikawa 2019). They can activate and mobilise immune cell subsets through chemoattraction, to treat disease or enhance immune responses in immunisation (Bobanga et al., 2013). Virus modification of chemokines presents unique combinations of properties which have utility as vaccine immune modulators or in immunotherapy of diseases, such as cancer or in autoimmunity (Vilgelm et al., 2019). Chemokines as adjuvants have been used to amplify protective immunity by their modulation of primer and effector functions of lymphocytes as well as their development (Mohan et al., 2018).

Interestingly, chemokines can also chemoattract regulatory leukocytes to suppress an immune response, as opposed to increasing a response. Therefore, chemokines can modulate the immune response by increasing or decreasing the immune response, depending on the chemokine receptors presenting on the interacting leukocyte sub-population. T-regs, have markers, such as transcription factor FOXP3, and subsets have been shown to express the CC chemokine receptos CCR4, CCR5, CCR6 or CCR8 (Bayry et al., 2008, Schlecker et al., 2012, Barsheshet et al,. 2017, Ohue and Nishikawa 2019, Snelgrove et al., 2019). Ligands to these receptors can chemoattract the T-regs to dampen an immune response. CCR5 can indicate the activation status of the T-reg subset, for example, for immune suppression during pregnancy to prevent rejection of the fetus, or similarly in graft vs host disease during transplantation (Kallikourdis, Andersen et al. 2007, Schlecker, Stojanovic et al. 2012). Attraction of CCR5 expressing T-regs by ligand secreting monocytic-myelocid derived suppressor cells, and thereby facilitated tumor growth via evading conventional T-cells (Schlecker, Stojanovic et al. 2012). Moreover, CCR6-expressing T-regs can modulate traffic into the Thymus and also inflamed endothelium (Snelgrove, Abeynaike et al. 2019, Peligero-Cruz, Givony et al. 2020). In addition, CCR4- and CCR8-expressing T-regs can modulate immunity in cancer by inhibiting anti-tumour immune responses, and CCR8-expressing T-regs modulate autoimmune encephalitis (Barsheshet, Wildbaum et al. 2017, Villarreal, L'Huillier et al. 2018, Ohue and Nishikawa 2019). WO2011/138785 discloses that CCL1 is specific for CCR8 on T-regs to treat for inflammatory, autoimmune, neuroinflammatory, such as encephalitis, and transplantation-related, graft vs host, diseases.

The effector part of the activated T-reg can include a number of mechanisms, which suppress the activation and function of other leukocytes (Lu, Barbi et al. 2017, Mohr, Atif et al. 2019). They can express co-inhibitory molecules, such as CTLA4 and LAG3, or secrete anti-inflammatory cytokines, as well as deplete growth factors, such as IL-2 via interaction with CD25. For example, CCR4 has been shown to be expressed on CD4+CD25+ T-regs (Iellem, Mariani et al. 2001). T-regs play a role in dampening down the immune response, to allow self-tolerance and also to prevent pathogenic over-responses to infection (Miyara and Sakaguchi 2007, Lu, Barbi et al. 2017, Luo and Li 2018). CCR4 binds both chemokine CCL17 and CCL22, which are secreted by dendritic cells (DC) and can chemoattract T-regs, where they enhance interactions between DC and CCR4+ T cells (Tang and Cyster 1999, Iellem, Mariani et al. 2001, Katou, Ohtani et al. 2001, Wu, Fang et al. 2001, Bayry, Tchilian et al. 2008, Snelgrove, Abeynaike et al. 2019). Different subsets of T-regs exist with CCR4 alone or with CCR6 (Mohr 2018). On the other hand, CCR5 has been shown to mark activated ‘effector’ T-regs (Kallikourdis, Andersen et al. 2007) effective for immune tolerance, for example, for the fetus or T-regs modulating inflamed skin (Ikebuchi, Fujimoto et al. 2019). T-regs can suppress DC maturation and co-stimulatory molecules expression, thereby decreasing their roles in activating T cells. Therefore, chemoattraction of T-regs can inhibit immune responses, while antagonising their recruitment during immunisation could increase immune responses that are induced by immunisation.

Modified ligands to individual receptors, for example, CCL1 directed to CCR8, have shown some utility in animal models in repressing an immune response (Barsheshet, Wildbaum et al. 2017). However, due to redundancy in the system, other receptors on T-regs could still function and there is a need to develop novel treatments acting via this group of regulatory cells.

Modified or mutated chemokines have also been developed and studied for their effects in treating diseases. For example, Met-CCL5 (also known as Met-RANTES) is an amino-terminally modified mature CCL5 protein, having a methionine residue added to the amino-terminal of the mature protein. Met-CCL5 was produced by recombinant expression of cDNA coding for mature CCL5 by E. coli (Proudfoot et al., 1996) and has been shown to antagonise migration of T-cells (Proudfoot et al, 1996) and antagonise monocyte migration and CCL5/RANTES-induced chemotaxis (Proudfoot et al, 1999). This modification of the amino-terminus of CCL5 disturbs its ability to fully activate certain signaling events, while not affecting other receptor activation states that lead to events such as receptor internalization (Proudfoot et al., 1999).

WO 96/17935 discloses that a modified version of CCL5/RANTES acts as an antagonist to CCL5/RANTES due to the presence of one or more N-terminal amino acids which are not present at the corresponding position in CCL5/RANTES. The modified CCL5/RANTES can be Met-RANTES, Leu-RANTES or Gln-RANTES.

CA2468790 discloses that mutants of CCL5, containing a single non-conservative substitution in a consensus sequence common to a subset of CC chemokines act as antagonists of CCL5. The human CC chemokines sharing this consensus sequence are CCL1, CCL2, CCL3, CCL4, CCL5, CCL7, CCL11, CCL13 and CCL15.

WO 2009/150433 discloses small molecule antagonists of CCR4, which enhance dendritic cell-mediated human T-cell proliferation.

KR 20210003550 discloses a composition of one or more of CCL4, CCL5, CCL20 and CCL21 for treating or preventing infertility, by improving the proliferation of inner membrane cells and reducing the expression of endoplasmic reticulum stress-inducing proteins.

US 2013344119 discloses an implantable composition comprising at least one of IL-8, MIP-3a (aka CCL20) and derivatives thereof for treating tissue damage or promoting tissue regeneration through an immune response.

The above examples have restricted use, and, therefore, there is a need to develop further specific and more effective immunotherapies, which are useful as treatments and vaccines for diseases by regulating the immune response. In particular, there is a need to develop further treatments and vaccines for the wide spectrum of diseases and conditions associated with an aberrant immune response, such as cancer (Karin 2018, Ohue and Nishikawa 2019), HIV infections (Catusse, Parry et al. 2007), autoimmune and inflammatory diseases (Mohr, Atif et al. 2019), diseases associated with runaway or excessive immune responses, such as cytokine storms, sepsis (Dong, Wang et al. 2020), and allergies (Mikhak et al., 2009).

### SUMMARY OF INVENTION

The inventor reasoned that the administering appropriate mixtures of agonist ligands of all T-regs could block an immune response and conversely with antagonist ligands could block the T-regs thereby stimulating an appropriate immune response. However, the correct mixture is not obvious, given in the CC chemokine family alone there are at least 23 chemokine receptors and 51 chemokine ligands (Hughes and Nibbs 2018, Mohan, Zhu et al. 2018). Further there are multiple subsets of T-regs with different tissue expression and multiple chemokine receptor utilisation both marking different T-reg subsets as well as their activation status (Lu, Barbi et al. 2017, Luo and Li 2018, Mohr, Atif et al. 2019). To decipher this, the inventor investigated herpesvirus genomes, as these viruses can modulate the chemokine system with homologues for chemokines and chemokine receptors. Human betaherpesvirus 6A, HHV-6A, encodes a chemokine homologue that binds and activates multiple chemokine receptors, U83A (Dewin, Catusse et al. 2006, Catusse, Parry et al. 2007). It was disclosed in U.S. Pat. Nos. 9,850,286 and 8,940,686 that the utility of full length U83A was to increase immune responses. Studies subsequent to this identify an endogenous form of this genome in human chromosomes integrated at the telomere, an inherited human adapted set of virus genes. These encode a human adapted form of the virus full-length chemokine homologue expressed in some people (Tweedy, Spyrou et al. 2015, Tweedy, Spyrou et al. 2016). The encoded chemokine receptor binding and signaling domains are retained and combines activity for CCR4, CCR5, CCR6 and CCR8 (Dewin, Catusse et al. 2006, Catusse, Parry et al. 2007). A novel spliced form has been identified by the inventor, and the cDNA for this, iciU83A-N, (SEQ ID NO: 16; referred to as VIT1 herein), encodes the chemokine receptor binding domain but does not retain the signaling domain. Thus, VIT1 could act as an antagonist to these chemokine receptors. These receptors are on immune stimulatory leukocytes as well as T-reg cells (Hughes and Nibbs 2018, Mohan, Zhu et al. 2018). Therefore, the inventor tested a human chemokine mixture to agonise these receptors and compared functional activities to the novel humanised virus gene cDNA antagonist to these receptors. Surprisingly, compared in a preclinical model for immunisation against HSV2 including combining the chemokines formulations with a well established, known immunogen, glycoprotein gD, the results for the novel antagonist, VIT1, stimulated protective immunity, whereas the agonist mixture of human chemokines (referred to herein as VTL3) inhibited immunity. This was reflected in the specific antibody response produced: the novel antagonist, VIT1, induced a specific antibody response, but the novel human chemokine agonist mixture, VTL3, completely blocked any antibody production. This is consistent with recent results defining CCR4, CCR5, CCR6 and CCR8 on T-reg populations, and the combined activity to all these receptors could eliminate recruitment of all the T-reg populations, thereby inhibiting helper and effector T-cell proliferation stimulating antibody and effector cytotoxic T-cell production. On the other hand, blocking chemoattraction of the T-regs with the novel antagonist allows conventional adaptive T-cell immunity to progress unchecked, thereby improving cellular immune responses. This efficient blocking of host cell immunity by the agonist mixture could treat autoimmune or inflammatory diseases with runaway pathogenic immune responses.

By modulating interactions with regulatory leukocytes by novel chemokines or chemokine mixtures, this can control aberrant immune responses, such as in autoimmune conditions or inflammation. Conversely, if these interactions are inhibited this can provide a transient stimulation by preventing dampening down of the immune response by regulatory leukocytes, with utility for vaccines for infectious disease or inducing immunological control of cancers.

Thus, the present invention arises because it has now been surprisingly found that mixtures of chemokines, or agonist or antagonist variants thereof, targeting CC chemokine receptors present on regulatory T-cells or other regulatory leukocytes, such as MDSC, monocytic myeloid-derived suppressor cells, can be used to regulate the immune response. For example, MDSC can secrete chemokines to attract T-regs (Schlecker et al., 2012). In particular, it has now been unexpectedly found that a mixture comprising a CCR5 agonist and a CCR4 and/or CCR6 agonist suppresses the immune response. This is surprising because it is not possible to predict how different chemokines and variants thereof will interact with any reasonable expectation of success. For illustration, different chemokine receptor chemokine interactions are present on different cancers and individual chemokines are ligands for both activator and regulatory T-cells (Korbecki, Grochans et al. 2020, Korbecki, Kojder et al. 2020; Hughes and Nibbs 2018; Mohan et al 2018). As discussed above, a human herpesvirus encodes a chemokine homologue U83A with specificities for these receptors interpreted as acting as an agonist, since it was demonstrated to effectively bind and signal to these receptors expressed separately (Dewin, Catusse et al. 2006, Catusse, Parry et al. 2007). Indeed, as disclosed, utility was based on use as an agonist to stimulate immune responses U.S. Pat. No. 9,850,286 B2 and U.S. Pat. No. 8,940,686. However, it has now been surprisingly found that a mixture of human chemokine agonists to these same set of receptors results instead in immune suppression. In addition, a novel cDNA from a human integrated HHV-6A genome (VIT1) has only the binding domain, and its utility has been shown to be the inverse, namely, to stimulate immune responses, in line with a dominant activity of antagonising the receptors on T-reg cells. Therefore, combined targeting of these receptors present on T-regs by agonists can chemoattract the combined sub-populations and lead to an inhibition of immune responses, while antagonism leads to the converse, namely immune stimulation.

The group of chemokine receptors targeted are on T-reg subsets. CCR5 is present on activated T-cell subsets including regulatory T-cells, while CCR4, CCR8 and CCR6 are present on regulatory T-cell subsets. While this set of chemokine receptors can define those present as characterised on regulatory T-cells or monocytic sub-populations, individually these receptors are also present on conventional T-cells (Hughes and Nibbs 2018, Mohan, Zhu et al. 2018). Thus, while not limited by theory, it is believed that the agonist or antagonist chemokine mixtures of the invention can modulate the immune response by either agonising receptors present on regulatory T-cells, thereby recruiting regulatory T-cells which act to dampen or inhibit the immune response, or by antagonising receptors present on regulatory T-cells, thereby preventing the recruitment of regulatory T-cells, leading to inducement or enhancement of the immune response.

Thus, in a first aspect of the invention here is provided a chemokine mixture comprising a first chemokine, or an agonist or antagonist variant thereof, which is suitable for binding to CCR4 and a second chemokine, or an agonist or antagonist variant thereof, which is suitable for binding to CCR5 and/or CCR6, wherein the chemokine mixture is suitable for modulating an immune response.

Preferably, the first chemokine or agonist or antagonist variant thereof comprises CCL17 or an agonist or antagonist variant thereof.

Conveniently, the second chemokine or agonist or antagonist variant thereof comprises CCL5 or CCL20 or an agonist or antagonist variant thereof.

Advantageously, the first chemokine or agonist or antagonist variant thereof comprises an amino acid sequence which has at least 70% sequence identity to SEQ ID NO: 5, or SEQ ID NO: 12 and/or the second chemokine or agonist or antagonist variant thereof comprises an amino acid sequence which has at least 70% sequence identity to one of SEQ ID NOs: 2, 8, 10, 14 or 26.

Preferably, the chemokine mixture comprises a third chemokine or an agonist or antagonist variant thereof, wherein the third chemokine or agonist or antagonist variant thereof is suitable for binding to a different one of CCR5 and CCR6 from the second chemokine or agonist or antagonist variant thereof.

Advantageously, the third chemokine or agonist or antagonist variant thereof comprises CCL20 or an agonist or antagonist variant thereof.

Conveniently, the third chemokine or agonist or antagonist variant thereof comprises an amino acid sequence which has at least 70% sequence identity to SEQ ID NO: 8 or SEQ ID NO: 14.

Preferably, a) the first chemokine comprises CCL17, the second chemokine comprises CCL5 or SEQ ID NO: 26, and the chemokine mixture comprises a third chemokine which comprises CCL20; or
b) the first chemokine comprises Met-CCL17, the second chemokine comprises Met-CCL5 or SEQ ID NO: 20, and the chemokine mixture comprises a third chemokine which comprises Met-CCL20, or
c) the first chemokine comprises CCL17, the second chemokine comprises Met-CCL5 or SEQ ID NO: 20, and the chemokine mixture comprises a third chemokine which comprises CCL20.

Conveniently, a) the first chemokine comprises the amino acid sequence of SEQ ID NO: 5, the second chemokine comprises the sequence of SEQ ID NO: 2 or 26 and the chemokine mixture comprises a third chemokine which comprises the amino acid sequence of SEQ ID NO: 8, or
b) the first chemokine comprises the amino acid sequence of SEQ ID NO:12, the second chemokine comprises the amino acid sequence of SEQ ID NO: 10 or 20 and the chemokine mixture comprises a third chemokine which comprises the amino acid sequence of SEQ ID NO: 14, or
c) the first chemokine comprises the amino acid sequence of SEQ ID NO: 5, the second chemokine comprises the amino acid sequence of SEQ ID NO: 10 or 20 and the third chemokine comprises the amino acid sequence of SEQ ID NO: 8.

In a second aspect of the invention, there is provided a polynucleotide mixture comprising a first polynucleotide and a second polynucleotide, wherein the first polynucleotide comprises a nucleic acid sequence which encodes a first chemokine, or an agonist or antagonist variant thereof, which is suitable for binding to CCR4 and the second polynucleotide comprises a nucleic acid sequence which encodes a second chemokine, or an agonist or antagonist variant thereof, which is suitable for binding to CCR5 and/or CCR6, wherein the polynucleotide mixture is suitable for modulating an immune response.

Preferably, the first polynucleotide comprises a nucleic acid sequence which encodes CCL17 or an agonist or antagonist variant thereof.

Advantageously, the second polynucleotide comprises a nucleic acid sequence which encodes CCL5 or an agonist or antagonist variant thereof, CCL20, or an agonist or antagonist variant thereof, or one of SEQ ID NOs: 20 and 26.

Conveniently, the first polynucleotide comprises a nucleic acid sequence which has at least 70% sequence identity to one of SEQ ID NOs: 6 and 13, and wherein the second polynucleotide comprises a nucleic acid sequence which has at least 70% sequence identity to one of SEQ ID NOs: 3, 9, 11, 15, 16-18 and 21-24.

Preferably, the second chemokine or functional variant thereof is suitable for binding to CCL5 and the polynucleotide mixture comprises a third polynucleotide, wherein the third polynucleotide comprises a nucleic acid sequence which encodes a third chemokine, or an agonist or antagonist variant thereof, wherein the third chemokine or agonist or antagonist variant thereof is suitable for binding to CCR6.

Advantageously, the third chemokine or agonist or antagonist variant thereof comprises CCL20 or a agonist or antagonist variant thereof.

Conveniently, the third polynucleotide comprises a nucleic acid sequence which has at least 70% sequence identity to SEQ ID NO: 13 or SEQ ID NO: 15.

Preferably, a) the first polynucleotide comprises a nucleic acid sequence which encodes CCL17, the second polynucleotide comprises a nucleic acid sequence which encodes CCL5 or which has an amino acid sequence which has at least 70% sequence identity to one of SEQ ID NO: 21-24, and the polynucleotide mixture comprises a third polynucleotide, wherein the third polynucleotide comprises a nucleic acid sequence which encodes CCL20; or
b) the first polynucleotide comprises a nucleic acid sequence which encodes Met-CCL17, the second polynucleotide comprises a nucleic acid sequence which encodes Met-CCL5 or which has at least 70% sequence identity to one of SEQ ID NOs: 16-18, and the polynucleotide mixture comprises a third polynucleotide, wherein the third polynucleotide comprises a nucleic acid sequence which encodes Met-CCL20, or
c) the first polynucleotide comprises an amino acid sequence which encodes CCL17, the second polynucleotide which encodes Met-CCL5 or which comprises an amino acid sequence which has at least 70% sequence identity to one of SEQ ID NOs: 16-18, and the polynucleotide mixture comprises a third polynucleotide, wherein the third polynucleotide comprises a nucleic acid sequence which encodes CCL20.

Advantageously, a) the first polynucleotide comprises a nucleic acid sequence which has at least 70% sequence identity to SEQ ID NO6, the second polynucleotide comprises a nucleic acid sequence which has at least 70% sequence identity to one of SEQ ID NOs: 3 and 21-24, and the polynucleotide mixture comprises a third polynucleotide, wherein the third polynucleotide comprises a nucleic acid sequence which at least 70% sequence identity to SEQ ID NO: 9; or
b) the first polynucleotide comprises a nucleic acid sequence which has at least 70% sequence identity to SEQ ID NO: 13, the second polynucleotide comprises a nucleic acid sequence which has at least 70% sequence identity to one of SEQ ID NOs: 11 and 16-18, and the polynucleotide mixture comprises a third polynucleotide, wherein the third polynucleotide comprises a nucleic acid sequence which has at least 70% sequence identity to SEQ ID NO: 15, or
c) the first polynucleotide comprises an amino acid sequence which has at least 70% sequence identity to SEQ ID NO: 6, the second polynucleotide comprises an amino acid sequence which has at least 70% sequence identity to one of SEQ ID NOs: 11 and 16-18, and the polynucleotide mixture comprises a third polynucleotide, wherein the third polynucleotide comprises a nucleic acid sequence which has at least 70% sequence identity to SEQ ID NO: 9.

Conveniently, a) the first polynucleotide comprises a nucleic acid sequence which has the sequence of SEQ ID NO: 6, the second polynucleotide comprises a nucleic acid sequence which has the sequence of SEQ ID NO: 3 and the polynucleotide mixture comprises a third polynucleotide, wherein the third polynucleotide comprises a nucleic acid sequence which has the sequence of SEQ ID NO: 9; or
b) the first polynucleotide comprises a nucleic acid sequence which has the sequence of one of SEQ ID Nos: 13, the second polynucleotide comprises a nucleic acid sequence which has the sequence of SEQ ID NO: 11, and the polynucleotide mixture comprises a third polynucleotide, wherein the third polynucleotide comprises a nucleic acid sequence which has the sequence of SEQ ID NO: 15, or
c) the first polynucleotide comprises a nucleic acid sequence which has the sequence of SEQ ID NO: 6, the second polynucleotide comprises a nucleic acid sequence which has the sequence of SEQ ID NO: 11 and the polynucleotide mixture comprises a third polynucleotide, wherein the third polynucleotide comprises a nucleic acid sequence which has the sequence of SEQ ID NO: 9.

In a third aspect of the invention, there is provided a composition comprising a first polynucleotide comprising a nucleic acid sequence which encodes a first chemokine or an agonist or antagonist variant thereof which is suitable for binding to one of CCR5, CCR4 and/or CCR6, and comprising a second chemokine or an agonist or antagonist variant thereof which is suitable for binding to a different one of CCR5, CCR4 and/or CCR6 from the first chemokine or agonist or antagonist thereof, wherein the composition is suitable for modulating an immune response.

Preferably, one of the first and second chemokines or agonist or antagonist variant thereof is suitable for binding to CCR4.

Advantageously, one of the first and second chemokines or agonist or antagonist variant thereof is suitable for binding to CCR4 and the other of the first and second chemokines or agonist or antagonist variant thereof is suitable for binding to CCR5 and/or CCR6.

Conveniently, the first polynucleotide encodes CCL5, CCL17, CCL20, or an agonist or antagonist variant thereof, or encodes an amino acid sequence having at least 70% sequence identity to one of SEQ ID Nos: 20 and 26.

Advantageously, the second chemokine or agonist or antagonist variant thereof comprises CCL5, CCL17, CCL20, or an agonist or antagonist variant thereof, or comprises an amino acid sequence which has at least 70% sequence identity to one of SEQ ID Nos: 20 and 26.

Preferably, the composition comprises a second polynucleotide which comprises a nucleic acid sequence which encodes a third chemokine or agonist or antagonist variant thereof, or the composition comprises a third chemokine or agonist or antagonist variant thereof, wherein the third chemokine or agonist or antagonist variant thereof is suitable for binding to a different one of CCR5, CCR4 and/or CCR6 from each of the first and second chemokines or agonist or antagonist variants thereof.

Advantageously, the third chemokine or agonist or antagonist variant thereof comprises CCL5, CCL17, CCL20, or agonist or antagonist variant thereof, or comprises an amino acid sequence which has at least 70% sequence identity to one of SEQ ID NOs: 20 and 26.

Conveniently, the first polynucleotide comprises a nucleic acid sequence which has at least 70% sequence identity to one of SEQ ID NOs: 3, 6, 9, 11, 13, 15-18 and 21-24.

Preferably, the composition comprises a second polynucleotide, wherein the second polynucleotide comprises a nucleic acid sequence which has at least 70% sequence identity to a different one of SEQ ID NOs: 3, 6, 9, 11, 13, 15-18 and 21-24 from the first polynucleotide.

Advantageously, the first chemokine or agonist or antagonist variant thereof comprises CCL5 or an agonist or antagonist variant thereof, or comprises an amino acid sequence having at least 70% sequence identity to one of SEQ ID NOs: 20 and 26, and the second chemokine or agonist or antagonist variant thereof comprises CCL17 or an agonist or antagonist variant thereof, and the composition comprises a third chemokine or agonist or antagonist variant thereof which comprises CCL20 or an agonist or antagonist variant thereof.

Preferably, the first chemokine or agonist or antagonist variant thereof comprises an amino acid sequence having at least 70% sequence identity to SEQ ID NO: 26, the second chemokine or agonist or antagonist variant thereof is CCL17 and the third chemokine or functional variant thereof is CCL20.

Advantageously, the first polynucleotide comprises a nucleic acid sequence having at least 70% sequence identity to one of SEQ ID NOs: 21-24, the second chemokine or agonist or antagonist variant thereof comprises an amino acid sequence having at least 70% sequence identity to SEQ ID NO: 5 and the third chemokine, or agonist or antagonist variant thereof, comprises an amino acid sequence having at least 70% sequence identity to SEQ ID NO: 8.

Conveniently, the first chemokine, or agonist or antagonist variant thereof, comprises an amino acid sequence having at least 70% sequence identity to SEQ ID NO: 20, the second chemokine or agonist or antagonist variant thereof is Met-CCL17 and the third chemokine or agonist or antagonist variant thereof is Met-CCL20.

Preferably, the first polynucleotide comprises a nucleic acid sequence having at least 70% sequence identity to one of SEQ ID NOs: 16-18, the second chemokine or agonist or antagonist variant thereof comprises an amino acid sequence having at least 70% sequence identity to SEQ ID NO: 12 and the third chemokine, or agonist or antagonist variant thereof, comprises an amino acid sequence having at least 70% sequence identity to SEQ ID NO: 14.

In a fourth aspect of the invention, there is provided a chemokine mixture or a polynucleotide mixture, wherein the chemokine mixture comprises a first chemokine, or an agonist or antagonist variant thereof, and a second chemokine, or agonist or antagonist variant thereof, or the polynucleotide mixture comprises a first polynucleotide which comprises a nucleic acid sequence which encodes a first chemokine, or an agonist or antagonist variant thereof, and a second polynucleotide which comprises a nucleic acid sequence which encodes a second chemokine, or an agonist or antagonist variant thereof, wherein the first chemokine, or agonist or antagonist variant thereof, is suitable for binding to a first CC chemokine receptor, wherein the first CC chemokine receptor is a marker of regulatory T-cells, and the second chemokine, or agonist or antagonist variant thereof, is suitable for binding to a second CC chemokine receptor, wherein the second CC chemokine receptor is a marker of activated and/or regulatory T-cells, and wherein the first and second chemokines, or agonist or antagonist variant thereof, are different from each other, for use in the treatment or prevention of a disease or disorder characterized by altered levels of CCR1, 4, 5, 6, and 8 or their binding chemokines, or a disease or disorder associated with a dysregulated immune response, or for use as an adjuvant.

In a fifth aspect of the invention, there is provided a composition comprising a first polynucleotide comprising a nucleic acid sequence which encodes a first chemokine, or an agonist or antagonist variant thereof, which is suitable for binding to a first CC chemokine receptor, wherein the first CC chemokine receptor is a marker of regulatory and/or activated T-cells, and comprising a second chemokine, or an agonist or antagonist variant thereof, which is suitable for binding to a second CC chemokine receptor, wherein the second CC chemokine receptor is a marker of regulatory and/or activated T-cells, wherein the first and second chemokines are different from each other, for use in the treatment or prevention of a disease or disorder characterized by altered levels of CCR1, 4, 5, 6, and 8 or their binding chemokines, or a disease/condition associated with a dysregulated immune response, or for use as an adjuvant.

Preferably, the disease or disorder is cancer, a viral infection, Alzheimer's disease, an autoimmune disease, an inflammatory disease or condition, an allergy or a disease or condition associated with an overactive immune system or runaway immune response.

Advantageously, in the fourth aspect, the first CC chemokine receptor is CCR4 and the second CC chemokine receptor is CCR5 or CCR6.

Conveniently, in the fourth aspect, the first chemokine comprises CCL17 or an agonist or antagonist variant thereof.

Preferably, in the fourth aspect, the second chemokine, or an agonist or antagonist variant thereof, is CCL5 or CCL20 or an agonist or antagonist variant thereof, or comprises an amino acid sequence which has at least 70% sequence identity to SEQ ID NO: 20 or SEQ ID NO: 26.

Advantageously, in the fourth aspect, the chemokine mixture comprises a third chemokine, or an agonist or antagonist variant thereof or the polynucleotide mixture comprises a third polynucleotide which comprises a nucleic acid which encodes a third chemokine or an agonist or antagonist variant thereof, respectively, wherein the second CC chemokine receptor is a marker of activated T-cells, and the third chemokine, or agonist or antagonist variant thereof, is suitable for binding to a third CC chemokine receptor, wherein the third CC chemokine receptor is a marker of regulatory T-cells and is different from the first CC chemokine receptor.

Conveniently, in the fourth aspect, the second CC chemokine receptor is CCR5 and the third CC chemokine receptor is CCR6.

Preferably, in the fourth aspect, the second chemokine, or agonist or antagonist variant thereof, comprises CCL5, or an agonist or antagonist variant thereof, and the third chemokine, or agonist or antagonist variant thereof, comprises CCL20, or a agonist or antagonist variant thereof.

Advantageously, in the fourth aspect, the first chemokine or agonist or antagonist variant thereof comprises an amino acid sequence which has at least 70% sequence identity to SEQ ID NO: 5, and the second chemokine or agonist or antagonist variant thereof comprises an amino acid sequence which comprises a sequence having at least 70% sequence identity to SEQ ID NO: 2, SEQ ID NO:8 or SEQ ID NO: 26, and the disease or disorder is an autoimmune disease, an inflammatory disease or condition, an allergy or a disease or condition associated with an overactive immune system or runaway immune response.

Conveniently, in the fourth aspect, the second chemokine, or agonist or antagonist variant thereof, comprises an amino acid sequence having at least 70% sequence identity to SEQ ID NO: 2 or 26 and wherein the chemokine mixture comprises a third chemokine, or an agonist or antagonist variant thereof, and the polynucleotide mixture comprises a third polynucleotide which comprises a nucleic acid sequence which encodes a third chemokine or an agonist or antagonist variant thereof, respectively, wherein the third chemokine or agonist or antagonist variant thereof comprises an amino acid sequence which has at least 70% sequence identity to SEQ ID NO: 8.

Preferably, in the fourth aspect, the first chemokine, or agonist or antagonist variant thereof, comprises an amino acid sequence which has at least 70% sequence identity to SEQ ID NO: 12, and the second chemokine, or agonist or antagonist variant thereof, comprises an amino acid sequence which comprises a sequence having at least 70% sequence identity to SEQ ID NO: 10, SEQ ID NO: 14 or SEQ ID NO: 20, for use in the treatment or prevention of a disease or disorder selected from cancer, a viral infection or Alzheimer's disease, or for use as an adjuvant.

Advantageously, in the fourth aspect, the second chemokine, or agonist or antagonist variant thereof, comprises an amino acid sequence having at least 70% sequence identity to SEQ ID NO: 10 or SEQ ID NO: 20, and wherein the chemokine mixture comprises a third chemokine, or an agonist or antagonist variant thereof, or the polynucleotide mixture comprises a third polynucleotide which comprises a nucleic acid sequence which encodes a third chemokine, or an agonist or antagonist variant thereof, respectively, wherein the third chemokine, or agonist or antagonist variant thereof, comprises an amino acid sequence which has at least 70% sequence identity to SEQ ID NO: 14.

Conveniently, in the fourth aspect, the first chemokine or agonist or antagonist variant thereof comprises an amino acid sequence which has at least 70% sequence identity to SEQ ID NO: 5 or SEQ ID NO: 8, and the second chemokine or agonist or antagonist variant thereof comprises an amino acid sequence which comprises a sequence having at least 70% sequence identity to SEQ ID NO: 10 or SEQ ID NO: 20, for use in the treatment or prevention of a disease or disorder selected from cancer, a viral infection or Alzheimer's disease, or for use as an adjuvant.

Preferably, in the fourth aspect, the first chemokine or agonist or antagonist variant thereof comprises an amino acid sequence which has at least 70% sequence identity to SEQ ID NO: 5, the second chemokine or agonist or antagonist variant thereof comprises an amino acid sequence which comprises a sequence having at least 70% sequence identity to SEQ ID NO: 10 or SEQ ID NO: 20, and the chemokine mixture comprises a third chemokine, or an agonist or antagonist variant thereof, or the polynucleotide mixture comprises a third polynucleotide which comprises a nucleic acid sequence which encodes a third chemokine, or an agonist or antagonist variant thereof, wherein the third chemokine or agonist or antagonist variant thereof, comprises an amino acid sequence which has at least 70% sequence identity to SEQ ID NO: 8.

Advantageously, in the fourth aspect, the first polynucleotide comprises a nucleic acid sequence which has at least 70% sequence identity to SEQ ID NO: 6, the second polynucleotide comprises a nucleic acid sequence which has at least 70% sequence identity to one of SEQ ID NOs: 3 and 21-24 and the third polynucleotide comprise a nucleic acid sequence which has at least 70% sequence identity to SEQ ID NO: 9.

Conveniently, in the fourth aspect, the first polynucleotide comprises a nucleic acid sequence which has at least 70% sequence identity to SEQ ID NO: 13, the second polynucleotide comprises a nucleic acid sequence which has at least 70% sequence identity to one of SEQ ID NOs: 11 and 16-18, and the third polynucleotide comprises a nucleic acid molecule which has at least 70% sequence identity to SEQ ID NO: 15.

Advantageously, in the fourth aspect, the first polynucleotide comprises a nucleic acid sequence which has at least 70% sequence identity to SEQ ID NO: 6, the second polynucleotide comprises a nucleic acid sequence which has at least 70% sequence identity to one of SEQ ID NOs: 11 and 16-18, and the third polynucleotide comprises a nucleic acid molecule which has at least 70% sequence identity to SEQ ID NO: 9.

Preferably, in the fifth aspect, each of the first and second CC chemokine receptors is, independently, CCR5, CCR4 or CCR6, wherein the first CC chemokine receptor is different from the second CC chemokine receptor.

Advantageously, in the fifth aspect, the first CC chemokine receptor is CCR5 and the second CC chemokine receptor is CCR4 or CCR6.

Conveniently, in the fifth aspect, the first chemokine, or agonist or antagonist variant thereof, comprises CCL5, or an agonist or antagonist variant thereof, or comprises an amino acid sequence having at least 70% sequence identity to one of SEQ ID NOs: 20 and 26, and the second chemokine or agonist or antagonist variant thereof comprises CCL17 or CCL20, or an agonist or antagonist variant thereof.

Preferably, in the fifth aspect, the composition comprises a second polynucleotide which comprises a nucleic acid sequence which encodes a third chemokine, or agonist or antagonist variant thereof, or the composition comprises a third chemokine, or agonist or antagonist variant thereof, wherein the third chemokine or agonist or antagonist variant thereof is suitable for binding to a third CC chemokine receptor which is a marker of activated and/or regulatory T-cells, wherein the third CC chemokine receptor is different from each of the first and second CC chemokine receptors.

Advantageously, in the fifth aspect, the first CC chemokine receptor is CCR5, the second CC chemokine receptor is one of CCR4 and CCR6 and the third chemokine receptor is the other of CCR4 and CCR6.

Conveniently, in the fifth aspect, the first chemokine, or agonist or antagonist variant thereof, comprises CCL5, or an agonist or antagonist variant thereof, or comprises an amino acid sequence which has at least 70% sequence identity to SEQ ID NO: 20 or 26, the second chemokine comprises one of CCL17 and CCL20, or an agonist or antagonist variant thereof, and the third chemokine comprises the other of CCL17 and CCL20, or an agonist or antagonist variant thereof.

Preferably, in the fifth aspect, the first chemokine comprises Met-CCL5, or an agonist or antagonist variant thereof, or comprises an amino acid sequence which has at least 70% sequence identity to SEQ ID NO: 20.

Advantageously, in the fifth aspect, the first polynucleotide comprises a nucleic acid sequence having at least 70% sequence identity to one of SEQ ID NOs: 10 and 16-18, the second chemokine comprises an amino acid sequence which has at least 70% sequence identity to one of SEQ ID NOs: 5 and 8, and the composition comprises third chemokine which has at least 70% sequence identity to the other of SEQ ID NOs: 5 and 8.

Conveniently, in the fifth aspect, the disease or disorder is selected from cancer, a viral infection or Alzheimer's disease, or the composition is for use as an adjuvant.

In a sixth aspect of the invention, there is provided a nucleic acid construct mixture comprising:
a first nucleic acid construct which comprises the first polynucleotide of the second aspect and a second nucleic acid construct which comprises the second polynucleotide of the second aspect;
a first nucleic acid construct which comprises the first polynucleotide of the third aspect and a second nucleic acid construct which comprises the second polynucleotide of the third aspect;
a first nucleic acid construct which comprises the first polynucleotide of the fourth aspect and a second nucleic acid construct which comprises the second polynucleotide of the fourth aspect; or a first nucleic acid construct which comprises the first polynucleotide of the fifth aspect and a second nucleic acid construct which comprises the second polynucleotide of the fifth aspect.

Preferably, the nucleic acid construct mixture comprises:
a first nucleic acid construct which comprises the first polynucleotide of the second aspect, a second nucleic acid construct which comprises the second polynucleotide of the second aspect and a third nucleic acid construct which comprises the third polynucleotide of the second aspect; or
a first nucleic acid construct which comprises the first polynucleotide of the fourth aspect, the second nucleic acid construct which comprises the polynucleotide of the fourth aspect and the third nucleic acid construct which comprises the polynucleotide of the fourth aspect.

In a seventh aspect of the invention, there is provided a nucleic acid construct comprising:
the first polynucleotide of the second aspect and the second polynucleotide of the second aspect;
the first polynucleotide of the third aspect and the second polynucleotide of claim 25 or 26;
the first polynucleotide of the fourth aspect and the second polynucleotide of the fourth aspect; or
the first polynucleotide of the fifth aspect and a second nucleic acid construct which comprises the second polynucleotide of the fifth aspect.

Preferably, the nucleic acid construct comprises:
the first polynucleotide of the second aspect, the second polynucleotide of the second aspect and the third polynucleotide of the second aspect; or
the first polynucleotide of the fourth aspect, the second the polynucleotide of the fourth aspect and the third polynucleotide of the fourth aspect.

In an eighth aspect of the invention, there is provided a host cell comprising the polynucleotide mixture of the second aspect, the polynucleotide mixture for use of the fourth aspect, the nucleic acid construct mixture of the sixth aspect, or the nucleic acid construct of the seventh aspect.

In a ninth aspect of the invention, there is provided a pharmaceutical formulation comprising the chemokine mixture of the first aspect, the polynucleotide mixture of the second aspect, the composition of the third aspect, the chemokine mixture or polynucleotide mixture for use of the fourth aspect, the composition for use of the fifth aspect, the nucleic acid construct mixture of the sixth aspect, the nucleic acid construct of the seventh aspect, or the host cell of the eighth aspect, and a pharmaceutically-acceptable carrier, excipient or diluent.

In a tenth aspect of the invention, there is provided a chemokine mixture of the first aspect; a polynucleotide mixture of the second aspect; a composition of the third aspect; a pharmaceutical composition comprising the chemokine mixture of the first aspect, the polynucleotide mixture of the second aspect, or the composition of the third aspect, and a pharmaceutically-acceptable carrier, diluent or excipient; a nucleic acid construct mixture of the sixth aspect; a nucleic acid construct of the seventh aspect; or a host cell of the eighth aspect; for use in the treatment or prevention of a disease or disorder characterized by altered levels of CCR1, 4, 5, 6, and 8 or their binding chemokines, or a disease or disorder associated with an aberrant immune response, or for use as an adjuvant.

In an eleventh aspect of the invention, there is provided a kit comprising the chemokine mixture of the first aspect, the polynucleotide mixture of the second aspect, the composition of the third aspect, the chemokine mixture or the polynucleotide mixture for use of the fourth aspect, the composition for use of the fifth aspect, the nucleic acid construct mixture of the sixth aspect, the nucleic acid construct of the seventh aspect, the host cell of the eighth aspect or the pharmaceutical formulation of the ninth aspect.

In a twelfth aspect of the invention, there is provided use of the chemokine mixture of the first aspect, the polynucleotide mixture of the second aspect, the composition of the third aspect, the nucleic acid construct mixture of the sixth aspect, the nucleic acid construct of the seventh aspect, or the host cell of the eighth aspect in the manufacture of a medicament.

Preferably, the medicament is for the treatment of a disorder characterised by altered levels of one or more of CCR1, CCR4, CCR5, CCR6 and CCR8 or their binding chemokines, or a disease or disorder associated with an aberrant immune response.

In a thirteenth aspect of the invention, there is provided a method of treating or preventing a disease or disorder characterized by altered levels of CCR1, 4, 5, 6, and 8 or their binding chemokines, or a disease or disorder associated with an aberrant immune response, wherein the method comprises administering the chemokine mixture of any one of claims 1-9, the polynucleotide mixture of any one of claims 10-19, the composition of any one of claims 20-33, the chemokine mixture or the polynucleotide mixture for use according to any one of claims 34 and 36-51, the composition for use according to any one of clams 35, 36 and -52-60, the nucleic acid construct mixture of claim 61 or 62 the nucleic acid construct of claim 63 or 64, the host cell of claim 65 or the pharmaceutical formulation of claim 66 to a patient in need thereof.

In a fourteenth aspect of the invention, there is provided use of the chemokine mixture of the first aspect, the polynucleotide mixture of the second aspect, the composition of the third aspect, the chemokine mixture or the polynucleotide mixture for use of the fourth aspect, the composition for use of the fifth aspect, the nucleic acid construct mixture of the sixth aspect, the nucleic acid construct of the seventh aspect, the host cell of the eighth aspect or the pharmaceutical formulation of the ninth aspect as an adjuvant in a patient in need thereof.

### Definitions

The term “protein”, as used herein, refers to amino acids in polymeric form of any length, linked together by peptide bonds.

The terms “nucleic acid”, “nucleic acid sequence”, “nucleotide”, “nucleic acid molecule” or “polynucleotide”, as used herein, are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The term “agonist variant”, as used herein, refers to a variant of a compound which binds to and activates the same receptor as the compound, thereby mimicking the biological effect of the compound.

The term “antagonist variant”, as used herein, refers to a variant of a compound which binds to, but does not activate, the same receptor as the compound, thereby preventing or reducing the biological effect of the compound.

The term “variant”, as used herein, refers to a variant nucleotide or amino acid sequence or part of the nucleotide or amino acid sequence (such as a fragment). The variant retains the ability to bind to the same receptor as the full non-variant sequence but may have an agonist or antagonist activity compared to the full non-variant. Also encompassed is a variant that is substantially identical, i.e. has only some sequence variations, for example in non-conserved residues, compared to the wild type sequences as shown herein and retains the non-variant binding activity. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Variants can also comprise one or more amino acid addition to or deletion from the amino acid sequence of the non-variant, and/or can comprise chemical modifications of the non-variant. Each of the proposed modifications is well within the routine skill in the art, as is determination of retention of biological activity of the encoded products. The variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence described herein. It is preferred that a variant has at least 70%, 75%, 80%, 85%, 90%, 95%, 96%, 97%, 98% or 99% sequence identity to the non-variant nucleic acid or amino acid sequence.

Alternatively, a variant may be a sequence that hybridises under stringent conditions to the nucleic or amino acid sequence. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

The term “immune response”, as used herein, refers in some embodiments to a T-cell- or B-cell-mediated immune response. A T-cell-mediated immune response occurs upon presentation of a peptide by major histocompatibility (MHC) molecules on the surface of cells, and in particular refers to activation of T-cells upon presentation of a peptide. A B-cell-mediated immune response refers, in particular, to the production of antibodies in response to recognition of an antigen.

The percentage “identity” between two sequences may be determined using the BLASTP algorithm version 2.2.2 (Altschul, Stephen F., Thomas L. Madden, Alejandro A. Schsffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

The term “pharmaceutical composition”, as used herein, means a pharmaceutical preparation suitable for administration to an intended human or animal subject for therapeutic purposes.

### BRIEF DESCRIPTION OF THE FIGURES

FIG. 1 shows the nucleic acid and corresponding amino acid sequence of an integrated iciHHV-6A iciU83A gene.

FIG. 2 shows the DNA sequence of spliced integrated iciHHV-6A U83A. This figure demonstrates our unexpected findings; splicing occurs as shown by cDNA analyses in transient gene expressed cells, through DR, direct repeat, TACC, and non-consensus splice donor/acceptor pairs despite a 3′ splice site proximal mutation, TGA-TGG, and that the non-synonymous SNP in the full length gene transforms this spliced product, unlike circulating virus, into mutation of the original spliced stop codon. Although thought to disrupt the non-consensus splicing, as this cDNA was not observed in samples from a set of donors (Tweedy et al., 2015, 2016), in vitro analyses in transfected cells (FIGS. 3 and 4) herein show that this unexpectedly now allows read through encoding 8 further amino acids with hydrophobic interactive properties as indicated.

FIG. 3 shows in vitro expression in cells with splicing of iciU83A into iciU83A-N. The gene iciU83A was cloned into a plasmid expression construct and transfected into HEK293 cells. Lanes 1-3 are negative controls, reaction mix with no oligonucleotide primers, reaction mix with oligonucleotide primers, reaction mix with oligonucleotide primers and water only template. Lanes 4-7 are one step RT-PCR reactions of total RNA extracted from the transfected cells primed using primers from the plasmid vector, pCMV (also with primers amplifying the iciU83A gene, not shown). Lanes 5 and 7 are untreated with DNase and show the residual DNA from transfection. Lane 4 and 6 are DNase treated. Lanes 4 and 5 include reverse transcriptase. Lane 5 shows the full length DNA and Lane 4 shows the expressed spliced cDNA product, iciU83A-N.

FIG. 4 shows in vitro expression in cells of iciU83A-N cDNA (SEQ ID NO: 16). The cDNA of iciU83A-N was cloned into a plasmid expression construct and transfected into HEK293 cells. Two days after transfection total RNA was extracted. The RNA was DNAse treated, lanes 2 and 3, then reverse transcriptase treated, lane 2, or untreated, lane 3. Negative control in lane 1 is water template only. Lane 4 shows the DNA markers.

FIG. 5 shows efficacy of immunisation with (A) iciU83A-N DNA construct, referred to as VTL1 (also referred to herein as VIT1 or or VIT), and (B) VTL3 protein, in an in vivo preclinical model of HSV2 evaluating protection from disease from acute infection to 14 days post virus challenge. The VTL1 or VTL3 were formulated with known immunogen gD, either as DNA with VTL1 DNA (VTL1016; SEQ ID NO: 17) or as protein with VTL3 protein (SEQ ID NOs: 2, 5 and 8). Comparisons were made to negative control (no vaccine) or positive control (gD protein with mpl/alum adjuvant). VTL1 DNA formulation showed almost complete protection while VTL3 protein formulation removes protection induced by the immunogen.

FIG. 6 shows efficacy of immunisation of known immunogen gD formulated as in FIG. 5 with (A) VIT1 DNA, and (B) VTL3 protein, in an in vivo preclinical model of HSV2 evaluating protection in individuals from total severity of disease from acute infection. With VIT1 (A), the total mean acute lesions show total elimination for 75% of the animals. This is similar to the positive control (gD protein mpl/alum immunisation). On the other hand, this effect is removed with VTL3 (SEQ ID NOs: 2, 5 and 8) (B); only 25% of the animals eliminate acute lesions and this is not significantly different from the negative control (no vaccine).

FIG. 7 shows efficacy of immunisation of a formulation with (A) VIT1 DNA and (B) VTL3 protein combined with known immunogen gD as DNA and protein respectively, in an in vivo preclinical model of HSV2 to evaluate effects of immunisation on protection from acute infection in. Comparisons were made to immunisation with positive control (gD protein plus adjuvant MPL plus alum). FIG. 7a shows significant reduction by the positive control immunisation in virus load in individual animals after virus challenge following immunisation, compared to the negative control (no vaccine). Evaluation of the immune modulators show that the VIT1 DNA (VTL1016; SEQ ID NO: 17) combined known immunogen gD DNA was effective in reducing viral load. FIG. 7b shows, in contrast, that immunisation with VTL3 blocked this effect and resulted in minimal reduction in virus load at day 2 post-infection, compared to the positive control (gD subunit protein).

FIG. 8 shows efficacy of immunisation of immunomodulators combined with known immunogen gD, (A) VIT DNA, and (B) VTL3 protein, evaluated in an in vivo preclinical model of HSV2 in protecting from recurrent disease. FIG. 8a shows that VIT provided protection from recurrent disease, with significant reduction in recurrent disease shown by cumulative recurrent lesion days in all animals after virus challenge following immunisation. Protection from recurrences in comparison to the negative control (no vaccine treatment) is only shown with the gD+VIT1 (SEQ ID NO: 17) combination, but was removed with the VIT1 removed. Therefore, VIT increased immune modulation with gD immunisation to provide protection from recurrent disease. Similarly, immunisation with the positive control (gD protein with adjuvant MPL/alum) also showed protection from recurrent disease. In contrast, FIG. 8b shows that immunisation of gD protein with the VTL3 chemokine formulation (SEQ ID NOs: 2, 5 and 8) abrogated protection from recurrent disease compared to the positive control, and rendered responses to the immunogen now similar to the negative control (no vaccine).

FIG. 9 shows efficacy of immunisation with (A) VIT1 DNA and (B) VTL3 protein, combined in formulation with known immunogen gD as DNA or protein, respectively, and evaluated in an in vivo preclinical model of HSV2 in providing protection from recurrent disease. FIG. 9a shows that immunisation with VIT1 resulted in a significant reduction in recurrent disease shown in individual animals as total severity of lesions which have recurred days 15-63 after virus challenge following immunisation. Significant protection from recurrences in comparison to the negative control (no vaccine treatment) is only shown with the gD+VIT1 (SEQ ID NO: 17) combination, but with the VIT1 removed, as immunisation with the gD immunogen alone shows reduced protection. FIG. 9b shows that immunisation using a gD protein with VTL3 completely removes any reduction in recurrent disease, rendering it similar to the negative control (no vaccine).

FIG. 10 shows efficacy of immunisation with immunomodulator (A) VIT1 DNA and (B) VTL3 protein, formulated with known immunogen gD as DNA or protein respectively, evaluated in an in vivo preclinical model of HSV2 in preventing asymptomatic recurrent shedding virus, as shown by total recurrences in individual animals, with the mean shown, as measured by quantitative DNA PCR (qPCR). FIG. 10a shows reduction in virus shedding only by immunisation with VIT1 DNA (VTL1016; SEQ ID NO: 17) with gD DNA. In contrast, FIG. 10b shows immunisation with VTL3 with gD, provides no protection and the result is similar to the negative control (no vaccine).

FIG. 11 shows efficacy of immunisation with immunomodulator (A) VIT1 DNA (VTL1016; SEQ ID NO: 17) and (B) VTL3 protein, formulated with known immunogen gD as DNA or protein, respectively, evaluated in an in vivo preclinical model of HSV2 in providing protection from establishment of latent infection in the dorsal root ganglia (DRG) of the animals, as measured by qPCR. FIG. 11a shows that the VIT1 formulation provided significant protection from establishment of latent infection in the DRG, halving that detected in the negative control, while FIG. 11b shows that the VTL3 formulation did not provide any protection.

FIG. 12 shows efficacy of immunisation using (A) VIT1 DNA (VTL1016; SEQ ID NO: 17) and (B) VTL3 protein, each formulated with known immunogen gD, evaluated in an in vivo preclinical model of HSV2 in providing protection from establishment of latent infection in the DRG of individual animals, as measured by qPCR. FIG. 12a shows that VIT1 provided significant protection, with over half of the animals being protected with no detectable DNA in 58% of the animals. FIG. 12b shows that VTL3 formulated with gD did not provide protection from establishment of latent infection in the DRG.

FIG. 13 shows efficacy of immunisation using (A) VIT1 DNA (VTL1016; SEQ ID NO: 17) and (B) VTL3 protein, each formulated with known immunogen gD, evaluated in an in vivo preclinical model of HSV2, in providing protection from establishment of latent infection in the spinal cord of the animals, as measured by qPCR. FIG. 13a shows that VIT1 provided significant protection, halving that detected in the negative control (no vaccine). FIG. 13b shows that VTL3 formulated with gD did not provide any protection compared to the negative control (no vaccine).

FIG. 14 shows efficacy of immunisation with (A) VIT1 DNA (VTL1016; SEQ ID NO: 17) and (B) VTL3 protein, each formulated with known immunogen gD, evaluated in an in vivo preclinical model of HSV2, in providing protection in individual animals from establishment of latent infection in the spinal cord of individual animals, as measured by qPCR. FIG. 14a shows that VIT1 provided significant protection in individual animals, compared to the negative control (no vaccine), with half of the animals being protected, with no detectable DNA in 50% of the animals. FIG. 14b shows that VTL3 did not provide any protection compared to the negative control (no vaccine).

FIG. 15 is a graph showing Herpes simplex virus type 2 (HSV-2) neutralising antibody titers in guinea pig serum after two intramuscular immunisations with the following vaccine formulations: gD protein combined with the immune regulator formulation, VTL3; gD DNA with VIT1; gD protein combined with MPL and alum; gD DNA with CCL5 DNA and no vaccine treatment. The dashed line shows the limit of detection. *** p<0.001 compared to no vaccine. Neutralising antibody was induced using gD combined with CCL5 or VIT1, compared to the negative control (no vaccine), but was completely inhibited by VTL3 protein, with the immune response remaining undetectable similar to the negative control (no vaccine).

### DETAILED DESCRIPTION

The invention relates, in general terms, to a chemokine mixture, comprising a first chemokine, or agonist or antagonist variant thereof, and a second chemokine, or agonist or antagonist thereof, targeting regulatory T-cells. The invention also relates to a polynucleotide mixture comprising a first polynucleotide which comprises a nucleic acid sequence encoding a first chemokine, or agonist or antagonist variant thereof, and a second polynucleotide which comprises a nucleic acid sequence which encodes a second chemokine, or agonist or antagonist variant thereof, targeting regulatory T-cells. The invention further relates to a composition comprising a first polynucleotide which comprises a nucleic acid sequence which encodes a first chemokine, or agonist or antagonist variant thereof, and a second chemokine, or agonist or antagonist variant thereof, targeting regulatory T-cells. The chemokine mixture, polynucleotide mixture and composition is each suitable for modulating an immune response. In each aspect of the invention, the first chemokine, or agonist or antagonist variant thereof, is suitable for binding to a first CC chemokine receptor, and the second chemokine, or agonist or antagonist variant thereof, is suitable for binding to a second CC chemokine receptor. Where present, the third chemokine is suitable for binding to a third CC chemokine receptor. The CC chemokine receptors are markers of activated and/or regulatory T-cells, and at least one of the CC chemokine receptors is a marker of regulatory T-cells. The descriptions below of the first and second chemokine, or agonist or antagonist variant thereof, and any further chemokine, or agonist or antagonist variant thereof, apply to the chemokines of, or encoded by, any of the chemokine mixtures, polynucleotide mixtures and compositions of the invention described herein. Thus, any combination of first and second, and optional third and further chemokines, or agonist or antagonist variant thereof, applies to any chemokine mixture, polynucleotide mixture and composition of the invention.

### Chemokines and CC Chemokine Receptors

Chemokines are a class of signalling proteins which are vital in cell migration, and, in particular, in the recruitment of immune cells, through chemotaxis. Human chemokines have been classified into four main subfamilies according to their amino acid composition, and particularly according to the first two cysteine residues of a conserved tetra-cysteine motif: CXC, CC, CX3C and C. However, other types of human chemokines, for example, a chemokine from a human chromosomally integrated endogenous form of human herpesvirus 6A (HHV-6A), referred to herein as iciHHV-6A.

Chemokines also include virokines, which are virally-encoded proteins that are secreted from the infected host cell and which can act as chemokine agonists or antagonists.

Chemokines exert their effects by binding to chemokine receptors expressed on the surface of leukocytes. In humans, there are 23 known chemokine receptors, which are all G protein-coupled receptors (GPCRs) containing seven transmembrane domains. The receptors are divided into four families depending on the type of chemokine they bind: CXC receptors (CXCR) bind CXC chemokines, CC receptors (CCR) bind CC chemokines, CX3C receptor 1 (CX3CR1) binds the only CX3C chemokine (CX3CL1) and XC receptor 1 (XCR1) which binds the two C chemokines. In particular, the CC receptor family consists of CCRs1-10, and their ligands are shown in Table 1 (adapted from Mohan et al. 2018 and Hughes and Nibbs 2018):

TABLE 1

| Receptor | Ligands |
|---|---|
| CCR1 | CCL3, CCL5, CCL6, CCL7, CCL8, CCL13, CCL16, CCL23 |
| CCR2 | CCL2, CCL7, CCL8, CCL12, CCL13 |
| CCR3 | CCL11, CCL26, CCL7, CCL13, CCL15, CCL24, CCL5, |
| | CCL8, CCL6 |
| CCR4 | CCL17, CCL22 |
| CCR5 | CCL3, CCL4, CCL5, CCL11, CCL13 |
| CCR6 | CCL20 |
| CCR7 | CCL19, CCL21 |
| CCR8 | CCL1, CCL4, (CCL17*) |
| CCR9 | CCL25 |
| CCR10 | CCL27, CCL28 |

*CCL17 was defined as binding CCR4 and CCR8, but other studies define only CCR4, and many TH2 cells express both receptors together (Mikhak et al, 2009; Fox et al 2006, Bernardini et al 1998).

### Modified Chemokines

Chemokines can also be modified to form agonist or antagonist variants. Protein modifications can include, amongst others, single amino acid additions, deletions or substitutions, or the addition of a chemical moiety. Met-CCL5 (Proudfoot et al., 1996; Proudfoot et al., 1999; WO 96/17935), AOP-CCL5 (Proudfoot et al., 1999), Gln-RANTES and Leu-RANTES (WO 96/17935) are examples of known modified chemokines. Met-CCL5, Gln-RANTES and Leu-RANTES all have an amino acid addition at the N-terminal thereof, while AOP-CCL5 is a chemical modification of CCL5.

In addition, the nucleotide sequence encoding a chemokine can be modified, for example, to promote expression of a functional protein, to modify the biological activity of the expressed protein, or to stabilise expression of the nucleotide sequence. Modifications of nucleotide sequences are well-known in the art, and Specific examples of such modifications are shown in SEQ ID NOs: 17, 18 and 21-24, herein.

Thus, any agonist variant of a chemokine, which is suitable for binding to and activating the CC chemokine receptor in question, or any antagonist variant of a chemokine, which is suitable for binding to, but does not activate, the CC chemokine receptor in question, may be used in the present invention. Methods of determining the binding affinity and biological activity of molecules are known, for example, using chemotaxis assays and receptor binding assays, such as those described in Proudfoot et al., 1996. Thus, the agonist and antagonist variants used in the present invention are not limited to the specific examples described herein.

### Mixtures

In the present invention, the first and second chemokine, or agonist or antagonist variant thereof, is suitable for binding to a CC chemokine receptor, wherein the first chemokine, or agonist or antagonist variant thereof, is suitable for binding a different CC chemokine receptor from the second chemokine, or agonist or antagonist variant thereof. The first and second chemokine, agonist or antagonist variant thereof, together, target regulatory T-cells, and either promote or prevent recruitment of regulatory T-cells, thereby modulating the immune response. The first chemokine, or agonist or antagonist variant thereof, may be suitable for binding to one of CCR5, CCR4 and/or CCR6 and the second chemokine, or agonist or antagonist variant thereof, may be suitable for binding to a different one of CCR5, CCR4 and/or CCR6. In some embodiments, one of the first and second chemokines, or agonist or antagonist variant thereof, is suitable for binding to CCR4 or CCR6. In some embodiments, the first chemokine, or agonist or antagonist variant thereof, is suitable for binding to CCR4, and the second chemokine, agonist or antagonist variant thereof, is suitable for binding to CCR5 and/or CCR6. In other embodiments, the first chemokine, or agonist or antagonist variant thereof, is suitable for binding to CCR4 and the second chemokine, or agonist or antagonist variant thereof, is suitable for binding to CCR5.

In some embodiments, the chemokine, or agonist or antagonist variant thereof, which is suitable for binding to CCR4 is also suitable for binding to CCR8. This applies to any chemokine, or agonist or antagonist variant thereof, which is suitable for binding to CCR4 in any of the mixtures and compositions described below.

In some embodiments, the chemokine mixture comprises a third chemokine, or agonist or antagonist variant thereof; the polynucleotide mixture comprises a third polynucleotide comprising a nucleic acid sequence encoding a third chemokine, or agonist or antagonist variant thereof; and/or the composition comprises a second polynucleotide which comprises a nucleic acid sequence which encodes a third chemokine, or agonist or antagonist variant thereof, or comprises a third chemokine, or agonist or antagonist variant thereof. The third chemokine, or agonist or antagonist variant thereof, is suitable for binding to a different one of CCR5, CCR4 and/or CCR6 from each of the first and second chemokines, or agonist or antagonist variant thereof. Thus, the first, second and third chemokines, or agonist or antagonist variant thereof, may be suitable for binding CCR5, CCR4 and/or CCR6 in any of the combinations shown in Table 2:

TABLE 2

| First chemokine, or | Second chemokine, or | Third chemokine, or |
|---|---|---|
| agonist or antagonist | agonist or antagonist | agonist or antagonist |
| variant thereof | variant thereof | variant thereof |
| CCR4 | CCR6 | CCR5 |
| CCR4 | CCR5 | CCR6 |
| CCR5 | CCR6 | CCR4 |
| CCR5 | CCR4 | CCR6 |
| CCR6 | CCR5 | CCR4 |
| CCR6 | CCR4 | CCR5 |

In preferred embodiments, the first chemokine, or agonist or antagonist variant thereof, is suitable for binding to CCR4. In some embodiments, the first chemokine, or agonist or antagonist variant thereof, is suitable for binding to each of CCR4 and CCR8.

In some embodiments, the chemokine, or agonist or antagonist variant thereof, suitable for binding to CCR5 comprises CCL5, Met-CCL5, SEQ ID NO: 19, SEQ ID NO: 20, SEQ ID NO: 25 or SEQ ID NO: 26, or an agonist or antagonist variant thereof. In some embodiments, the chemokine, or agonist or antagonist variant thereof, suitable for binding to CCR4 comprises CCL17, Met-CCL17 or an agonist or antagonist variant thereof. In some embodiments, the chemokine, or agonist or antagonist variant thereof, suitable for binding to CCR6 comprises CCL20, Met-CCL20 or an agonist or antagonist variant thereof. Any of these chemokines, or agonist or antagonist variant thereof, can be used in any of the combinations of the first and second chemokine, or agonist or antagonist variant thereof, and in any of the combinations of Table 2. Thus, in some embodiments, the first chemokine, or agonist or antagonist variant thereof, comprises CCL17, Met-CCL17, or an agonist or antagonist variant thereof, and the second chemokine, or agonist or antagonist variant thereof, comprises, CCL5, Met-CCL5, SEQ ID NO: 19, SEQ ID NO: 20, SEQ ID NO: 25, SEQ ID NO: 26, CCL20 or Met-CCL20, or an agonist or antagonist thereof. In some embodiments, the first chemokine, or agonist or antagonist variant thereof, comprises CCL17, Met-CCL17, or an agonist or antagonist variant thereof, the second chemokine, or agonist or antagonist variant thereof, comprises CCL5 Met-CCL5, SEQ ID NO: 19, SEQ ID NO: 20, SEQ ID NO: 25 or SEQ ID NO: 26, or an agonist or antagonist variant thereof, and the third chemokine comprises CCL20, Met-CCL20 or an agonist or antagonist variant thereof.

In some embodiments, the chemokine, or agonist or antagonist variant thereof, suitable for binding to CCR5 comprises an amino acid sequence which has at least 70%, 75%, 80%, 85%, 90%, 95%, 96%, 97%, 98%, 99% or 100% sequence identity to one of SEQ ID NOs: 1, 2, 10, 12, 14, 19, 20, 25 and 26. In some embodiments, the amino acid sequence has at least 70%, 75%, 80%, 85%, 90%, 95%, 96%, 97%, 98%, 99% or 100% sequence identity to one of SEQ ID NOs: 2, 10, 20 and 26. In some embodiments, the amino acid sequence has at least 90% sequence identity to one of SEQ ID NOs: 2, 10, 20 and 26. In some embodiments, the amino acid sequence has at least 95% sequence to one of SEQ ID NOs: 2, 10, 20 and 26. In some embodiments, the amino acid sequence has at least 99% sequence identity to one of SEQ ID NOs: 2, 10, 20 and 26. In some embodiments, the amino acid sequence is identical to one of SEQ ID NOs: 2, 10, 20 and 26.

In some embodiments, the chemokine, or agonist or antagonist variant thereof, suitable for binding to CCR4 comprises an amino acid sequence which has at least 70%, 75%, 80%, 85%, 90%, 95%, 96%, 97%, 98%, 99% or 100% sequence identity to one of SEQ ID NOs: 4, 5 and 12. In some embodiments, the amino acid sequence has at least 90% sequence identity to one of SEQ ID NOs: 5 and 12. In some embodiments, the amino acid sequence has at least 95% sequence to one of SEQ ID NOs: 5 and 12. In some embodiments, the amino acid sequence has at least 99% sequence identity to one of SEQ ID NOs: 5 and 12. In some embodiments, the amino acid sequence is identical to one of SEQ ID NOs: 5 and 12.

In some embodiments, the chemokine, or agonist or antagonist variant thereof, suitable for binding to CCR6 comprises an amino acid sequence which has at least 70%, 75%, 80%, 85%, 90%, 95%, 96%, 97%, 98%, 99% or 100% sequence identity to one of SEQ ID NOs: 7, 8 and 14. In some embodiments, the amino acid sequence has at least 90% sequence identity to one of SEQ ID NOs: 8 and 14. In some embodiments, the amino acid sequence has at least 95% sequence to one of SEQ ID NOs: 8 and 14. In some embodiments, the amino acid sequence has at least 99% sequence identity to one of SEQ ID NOs: 8 and 14. In some embodiments, the amino acid sequence is identical to one of SEQ ID NOs: 8 and 14.

Any of the above amino acid sequences and percentage sequence identities thereto can be used in any of the combinations of the first and second chemokine, and in any of the combinations of Table 2. Thus, in some embodiments, the first chemokine, or agonist or antagonist variant thereof, comprises an amino acid sequence which has at least 70%, 75%, 80%, 85%, 90%, 95%, 96%, 97%, 98%, 99% or 100% sequence identity to one of SEQ ID NOs: 5 and 12, and the second chemokine, or agonist or antagonist variant thereof, comprises an amino acid sequence which has at least 70%, 75%, 80%, 85%, 90%, 95%, 96%, 97%, 98%, 99% or 100% sequence identity to one of SEQ ID NOs: 2, 8, 10, 12, 14, 20 and 26. In some embodiments, the first chemokine, or agonist or antagonist variant thereof, comprises an amino acid sequence which has at least 70%, 75%, 80%, 85%, 90%, 95%, 96%, 97%, 98%, 99% or 100% sequence identity to one of SEQ ID NOs: 5 and 12, the second chemokine, or agonist or antagonist variant thereof, comprises an amino acid sequence which has at least 70%, 75%, 80%, 85%, 90%, 95%, 96%, 97%, 98%, 99% or 100% sequence identity to one of SEQ ID NOs: 2, 10, 20 and 26, and the third chemokine or agonist or antagonist variant thereof, comprises an amino acid sequence which has at least 70%, 75%, 80%, 85%, 90%, 95%, 96%, 97%, 98%, 99% or 100% sequence identity to one of SEQ ID NOs: 8 and 14. Preferably, said amino acid sequence of each chemokine, or agonist or antagonist variant thereof, has at least 90%, 95% or 99%, and more preferably 100%, sequence identity to the SEQ ID NO in question.

### Agonist Chemokine Mixtures

In some embodiments, each of the first and second chemokine, or variant thereof, is an agonist of the respective CC chemokine receptor. In other words, in some embodiments, the first and second chemokine, or variant thereof, binds to and activates the relevant CC chemokine receptor, thereby recruiting regulatory T-cells. Thus, in some embodiments, the first chemokine or variant thereof is a CCR4 agonist and the second chemokine or variant thereof is a CCR5 or a CCR6 agonist. In some embodiments, the first chemokine or variant thereof is a CCR4 agonist, the second chemokine or variant thereof is a CCR5 agonist and the third chemokine or variant thereof is a CCR6 agonist. Methods of determining the binding affinity and biological activity of molecules are known, for example, using chemotaxis assays and receptor binding assays, such as those described in Proudfoot et al., 1996. Thus, the CC chemokine receptor agonists used in the present invention are not limited to the specific examples described herein.

In some embodiments, the first chemokine, or agonist variant thereof, comprises CCL17, and the second chemokine, or agonist variant thereof, comprises CCL5, SEQ ID NO: 26, CCL17 or CCL20. In some embodiments, the first chemokine, or agonist variant thereof, comprises CCL17, and the second chemokine, or agonist variant thereof, comprises CCL5 or CCL20. In some embodiments, the first chemokine, or agonist variant thereof, comprises CCL17 and the second chemokine, or agonist variant thereof, comprises SEQ ID NO: 26 or CCL20. In some embodiments, the first chemokine, or agonist variant thereof, comprises CCL17, the second chemokine, or agonist variant thereof, comprises CCL5 or SEQ ID NO: 26 and the third chemokine, or agonist variant thereof, comprises CCL20.

In some embodiments, the first chemokine, or agonist variant thereof, comprises an amino acid sequence which has at least 70%, 75%, 80%, 85%, 90%, 95%, 96%, 97%, 98%, 99% or 100% sequence identity to SEQ ID NO: 5 and the second chemokine, or agonist variant thereof, comprises an amino acid sequence which has at least 70%, 75%, 80%, 85%, 90%, 95%, 96%, 97%, 98%, 99% or 100% sequence identity to one of SEQ ID NOs: 2, 8 and 26. In some embodiments, the first chemokine, or agonist variant thereof, comprises an amino acid sequence which has at least 70%, 75%, 80%, 85%, 90%, 95%, 96%, 97%, 98%, 99% or 100% sequence identity to SEQ ID NO: 5, and the second chemokine, or agonist variant thereof, comprises an amino acid sequence which has at least 70%, 75%, 80%, 85%, 90%, 95%, 96%, 97%, 98%, 99% or 100% sequence identity to one of SEQ ID NOs: 2 and 8. In some embodiments, the first chemokine or agonist thereof comprises an amino acid sequence which has at least 70%, 75%, 80%, 85%, 90%, 95%, 96%, 97%, 98%, 99% or 100% sequence identity to SEQ ID NO: 5, and the second chemokine, or agonist variant thereof, comprises an amino acid sequence which has at least 70%, 75%, 80%, 85%, 90%, 95%, 96%, 97%, 98%, 99% or 100% sequence identity to one of SEQ ID NOs: 8 and 26. In some embodiments, the first chemokine, or agonist variant thereof, comprises an amino acid sequence which has at least 70%, 75%, 80%, 85%, 90%, 95%, 96%, 97%, 98%, 99% or 100% sequence identity to SEQ ID NO: 5, the second chemokine, or agonist variant thereof, comprises an amino acid sequence which has at least 70%, 75%, 80%, 85%, 90%, 95%, 96%, 97%, 98%, 99% or 100% sequence identity to one of SEQ ID NOs: 2 and 26, and the third chemokine or agonist variant thereof comprises an amino acid sequence which has at least 70%, 75%, 80%, 85%, 90%, 95%, 96%, 97%, 98%, 99% or 100% sequence identity to SEQ ID NO: 8.

In some embodiments, the first chemokine or agonist variant thereof comprises an amino acid sequence which consists of SEQ ID NO: 5 and the second chemokine or agonist variant thereof comprises an amino acid sequence which consists of SEQ ID NO: 2, 8 or 26. In some embodiments, the first chemokine or agonist variant thereof comprises an amino acid sequence which consists of SEQ ID NO: 5 and the second chemokine or agonist thereof comprises an amino acid sequence which consists of SEQ ID NO: 2 or 8. In some embodiments, the first chemokine or agonist variant thereof comprises an amino acid sequence which consists of SEQ ID NO: 25 and the second chemokine or agonist thereof comprises an amino acid sequence which consists of SEQ ID NO: 8 or 26. In some embodiments, the first chemokine or agonist variant there of comprises an amino acid sequence which consists of SEQ ID NO: 5, the second chemokine or agonist variant thereof comprises an amino acid sequence which consists of SEQ ID NO: 2 or 26 and the third chemokine or agonist variant thereof comprises an amino acid sequence which consists of SEQ ID NO: 8. In some embodiments, the first chemokine consists of SEQ ID NO: 5, the second chemokine consists of SEQ ID NO: 2 or 26 and the third chemokine consists of SEQ ID NO: 8. One embodiment, in which the first chemokine consists of SEQ ID NO: 5, the second chemokine consists of SEQ ID NO: 2 and the third chemokine consists of SEQ ID NO: 8, is referred to herein as “VTL3”. In particular, FIGS. 5b, 6b, 7b, 8b, 9b, 10b, 11b, 12b, 13b, 14b, 15b and 16b show that VTL3 abrogated any protection against viral challenge, and FIG. 17 shows that VTL3 inhibited an antibody response.

It has been surprisingly found that a mixture of chemokines which can target regulatory T-cells have utility to inhibit an immune response. This is surprising because the interactions between chemokines can have different effects, which are not predictable, as discussed in Proudfoot et al., 2016. Moreover, individual agonists of the individual receptors can be immune stimulatory, for example, by chemoattracting dendritic cells or helper T cell subsets TH17 (CCR6 with CCL20) or skin homing effector TH2 cells (CCR4 or CCR8 with CCL17) (Hughes and Nibbs 2018; Mohan et al 2018). With specific regard to VTL3, it is shown herein that, while a CCR5 agonist, such as CCL5, induces an immune response, the combination of an agonist mixture targeting CCR5, CCR4, CCR8 and CCR6 inhibits an immune response. Conversely, a novel antagonist molecule, VTL1 (also referred to herein as VIT1), which is specific for all of CCR5, CCR4, CCR8 and CCR6, induces an immune response. Thus, the combination of VTL1 with other antagonists is expected to have a opposite effect to an agonist mixture targeting these four CC chemokine receptors.

### Antagonist Chemokine Mixtures

In some embodiments, each of the first and second chemokine, or variant thereof, is an antagonist of the respective CC chemokine receptor. In other words, in some embodiments, the first and second chemokine, or variant thereof, binds to, but does not activate, the relevant CC chemokine receptor, thereby inhibiting the recruitment of regulatory T-cells and, consequently, permitting or enhancing an immune response. Thus, in some embodiments, the first chemokine or variant thereof is a CCR4 antagonist and the second chemokine or variant thereof is a CCR5 antagonist or a CCR6 antagonist. In some embodiments, first chemokine or variant thereof is a CCR4 antagonist, the second chemokine or variant thereof is a CCR5 antagonist and the third chemokine or variant thereof is a CCR6 antagonist.

It is expected that mixtures of antagonists of CC chemokine receptors, targeting activated regulatory T-cells, will be effective in inducing, enhancing or permitting an immune response. In particular, and as discussed above, the Figures show that the chemokine mixture VTL3, which is made up of CC chemokine receptor agonists which target both conventional and activated regulatory T-cells, surprisingly inhibits an immune response. This mixtures targets all T-reg subsets via the combined chemokine receptor specificity and the net effect is inhibitory. It is therefore expected that a mixture which contains one or more antagonists of the chemokines contained in VTL3, and particularly a mixture which contains antagonists of all of the chemokines contained in VTL3, will have the opposite effect, namely to increase or permit an immune response.

As discussed above, a number of CC chemokine receptor antagonists are known, for example, Met-CCL5 (Proudfoot et al., 1996; Proudfoot et al., 1999; WO 96/17935), AOP-CCL5 (Proudfoot et al., 1999), Gln-RANTES and Leu-RANTES (WO 96/17935). These four specific CCL5 variants are N-terminally modified and, as it is the N-terminal of CC chemokines which interacts with CC chemokine receptors (as discussed above), it is expected that N-terminal modifications of chemokines will alter activity and the Met N-terminal modification, in particular, transitioning an agonist to antagonist, as demonstrated by Met-CCL5. Thus, in the present invention, antagonistic variants of chemokines include N-terminally-modified chemokines, such as -terminally met-modified chemokines.

In addition, it is shown herein that VTL1 (also referred to herein as VIT1) is an antagonist of CC chemokine receptors, and specifically of CCR5, together with CCR4, CCR6 and CCR8. This is because VTL1 retains binding specificity but lacks any signalling domains. VTL1 is specific for the same receptors as VTL3. The encoded signaling domain has been previously characterised (Dewin et al., 2006; Catusse et al 2007). The novel cDNA encoding VTL1 has the signaling domain removed by splicing, but retains the binding domain. Furthermore, VTL1 has been shown to protect against HSV2 infection by vaccination together with a known immunogen (FIGS. 5a, 6a, 7a, 8a, 9a, 10a, 11a, 12a, 13a, 14a, 15). As mentioned above, this is because VTL1 retains the binding domain which includes specificity for CCR5, as well as CCR4, CCR6 and CCR8, and so can act as antagonist by binding without signaling. Thus, it is expected that VTL1 includes CCR5 antagonist properties that are the same or similar to Met-CCL5. Therefore, VTL1, with the combined chemokine receptors' activity, could inhibit chemoattraction of the complete T-reg populations, thereby altering the balance to conventional T cell proliferation, leading more efficient antibody and effector T cell production.

It is also expected that Met-CCL17 and Met-CCL20 will antagonise CCR4 and CCR6, respectively. In particular, CCL17 and CCL20 have a similar structure to CCL5, and, as discussed above, it is the N-terminus of each of these chemokines which interacts with the relevant receptor. Met-CCL5 comprises an additional methionine residue at its N-terminus compared to wild-type CCL (Proudfoot et al, 1996), and it is expected that the same modification of CCL17 and CCL20 will have similar effects as methionylation of CCL5, namely, to convert CCL17 and CCL20 into antagonists of CCR4 and CCR6, respectively.

Thus, any known CC chemokine receptor antagonist, which is suitable for binding to, but does not activate, the CC chemokine receptor in question may be used. In addition, methods of determining the binding affinity and biological activity of molecules are known, for example, using chemotaxis assays and receptor binding assays, such as those described in Proudfoot et al., 1996. Thus, the CC chemokine receptor antagonists used in the present invention are not limited to the specific examples described herein.

Furthermore, when the mixture comprises a CCR5 antagonist, it is expected that the CCR5 antagonist can be combined with a CCR4 and/or a CCR6 agonist and still antagonise prevent or reduce an immune response. This is because CCR5 is a marker of activated T-cells, such that a CCR5 antagonist will prevent the recruitment of activated regulatory T-cells. Thus, although the CCR4 and/or the CCR6 agonist promotes the recruitment of regulatory T-cells, these are not expected to be, or to become, activated regulatory T-cells, such that the regulatory T-cells will not be effective in dampening the immune response.

The first and second chemokines, and optional third chemokine, or their antagonist variants thereof, can be combined in any of the combinations shown in Table 3. Each chemokine or variant thereof comprises the specified sequence and can comprise further modifications.

TABLE 3

| First chemokine or | Second chemokine or | Third chemokine or |
|---|---|---|
| agonist or antagonist | agonist or antagonist | agonist or antagonist |
| variant thereof | variant thereof | variant thereof |
| Met-CCL17 | Met-CCL5 | n/a |
| Met-CCL17 | SEQ ID NO: 20 | n/a |
| Met-CCL17 | Met-CCL20 | n/a |
| CCL17 | Met-CCL5 | n/a |
| CCL17 | SEQ ID NO: 20 | n/a |
| Met-CCL17 | Met-CCL5 | Met-CCL20 |
| CCL17 | Met-CCL5 | Met-CCL20 |
| Met-CCL17 | Met-CCL5 | CCL20 |
| CCL17 | Met-CCL5 | CCL20 |
| CCL17 | SEQ ID NO: 20 | Met-CCL20 |
| Met-CCL17 | SEQ ID NO: 20 | CCL20 |
| CCL17 | SEQ ID NO: 20 | CCL20 |
| Met-CCL17 | Met-CCL5 | Met-CCL20 |
| Met-CCL17 | Met-CCL20 | Met-CCL5 |
| Met-CCL17 | Met-CCL20 | SEQ ID NO: 20 |
| Met-CCL17 | SEQ ID NO: 20 | Met-CCL20 |

In some embodiments, the first chemokine, or antagonist variant thereof, comprises Met-CCL17, the second chemokine, or antagonist variant thereof, comprises Met-CCL5 or SEQ ID NO: 20. In some embodiments, the first chemokine, or antagonist variant thereof, comprises CCL17, and the second chemokine, or antagonist variant thereof, comprises Met-CCL5 or SEQ ID NO: 20. In some embodiments, the first chemokine, or antagonist variant thereof, comprises Met-CCL17, the second chemokine or antagonist variant thereof comprises Met-CCL20 or SEQ ID NO: 20 and the third chemokine or antagonist variant thereof comprises Met-CCL20. In some embodiments, the first chemokine or antagonist variant thereof is CCL17, the second chemokine or antagonist variant thereof is Met-CCL5 or SEQ ID NO: 20, and the third chemokine or antagonist variant thereof is CCL20.

In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, and the second chemokine, or antagonist variant thereof, comprises an amino acid sequence which has at least 70%, 75%, 80%, 85%, 90%, 95%, 96%, 97%, 98%, 99% or 100% sequence identity to one of SEQ ID NOs: 10, 14 and 20. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, and the second chemokine, or antagonist variant thereof, comprises an amino acid sequence which has at least 70%, 75%, 80%, 85%, 90%, 95%, 96%, 97%, 98%, 99% or 100% sequence identity to one of SEQ ID NOs: 10 and 20. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, and the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 14.

In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 5, and the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 10 or 20.

In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 10, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 14. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 20, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 14.

In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 5, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 10, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 14. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 10, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 8. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 5, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 10, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 8. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 5, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 20, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 14. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 20, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 8. In some embodiments, the first chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 5, the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 20, and the third chemokine or antagonist variant thereof comprises an amino acid sequence which has at least 70%, 75%, 80%, 85%, 90%, 95%, 96%, 97%, 98%, 99% or 100% sequence identity to SEQ ID NO: 8.

In some embodiments, the first chemokine or antagonist variant thereof comprises an amino acid sequence which consists of SEQ ID NO: 12 and the second chemokine or antagonist variant thereof comprises an amino acid sequence which consists of SEQ ID NO: 10, 14 or 20. In some embodiments, the first chemokine or antagonist variant thereof comprises an amino acid sequence which consists of SEQ ID NO: 12 and the second chemokine or antagonist thereof comprises an amino acid sequence which consists of SEQ ID NO: 10 or 20. In some embodiments, the first chemokine or antagonist variant thereof comprises an amino acid sequence which consists of SEQ ID NO: 12 and the second chemokine or antagonist thereof comprises an amino acid sequence which consists of SEQ ID NO: 14. In some embodiments of the mixtures above, each chemokine or antagonist variant thereof consists of the specified sequence.

In some embodiments, the first chemokine or antagonist variant thereof comprises an amino acid sequence which consists of SEQ ID NO: 5 and the second chemokine or antagonist variant thereof comprises an amino acid sequence which consists of SEQ ID NO: 10 or 20. In some embodiments of the mixture above, each chemokine or antagonist variant thereof consists of the specified sequence.

In some embodiments, the first chemokine or antagonist variant thereof comprises an amino acid sequence which consists of SEQ ID NO: 12, the second chemokine or antagonist variant thereof comprises an amino acid sequence which consists of SEQ ID NO: 10, and the third chemokine or antagonist variant thereof comprises an amino acid sequence which consists of SEQ ID NO: 14. In some embodiments, the first chemokine or antagonist variant thereof comprises an amino acid sequence which consists of SEQ ID NO: 12, the second chemokine or antagonist variant thereof comprises an amino acid sequence which consists of SEQ ID NO: 20, and the third chemokine or antagonist variant thereof comprises an amino acid sequence which consists of SEQ ID NO: 14. In some embodiments of the mixtures above, each chemokine or antagonist variant thereof consists of the specified sequence.

In some embodiments, the first chemokine or antagonist variant thereof comprises an amino acid sequence which consists of SEQ ID NO: 5, the second chemokine or antagonist variant thereof comprises an amino acid sequence which consists of SEQ ID NO: 10 and the third chemokine or antagonist variant thereof comprises an amino acid sequence which consists of SEQ ID NO: 14. In some embodiments, the first chemokine or antagonist variant thereof comprises an amino acid sequence which consists of SEQ ID NO: 5, the second chemokine or antagonist variant thereof comprises an amino acid sequence which consists of SEQ ID NO: 20 and the third chemokine or antagonist variant thereof comprises an amino acid sequence which consists of SEQ ID NO: 14. In some embodiments, the first chemokine or antagonist variant thereof comprises an amino acid sequence which consists of SEQ ID NO: 12, the second chemokine or antagonist variant thereof comprises an amino acid sequence which consists of SEQ ID NO: 10 and the third chemokine or antagonist variant thereof comprises an amino acid sequence which consists of SEQ ID NO: 8. In some embodiments, the first chemokine or antagonist variant thereof comprises an amino acid sequence which consists of SEQ ID NO: 12, the second chemokine or antagonist variant thereof comprises an amino acid sequence which consists of SEQ ID NO: 20 and the third chemokine or antagonist variant thereof comprises an amino acid sequence which consists of SEQ ID NO: 8. In some embodiments, the first chemokine or antagonist variant thereof comprises an amino acid sequence which consists of SEQ ID NO: 5, the second chemokine or antagonist variant thereof comprises an amino acid sequence which consists of SEQ ID NO: 10 and the third chemokine or antagonist variant thereof comprises an amino acid sequence which consists of SEQ ID NO: 8. In some embodiments, the first chemokine or antagonist variant thereof comprises an amino acid sequence which consists of SEQ ID NO: 5, the second chemokine or antagonist variant thereof comprises an amino acid sequence which consists of SEQ ID NO: 20 and the third chemokine or antagonist variant thereof comprises an amino acid sequence which consists of SEQ ID NO: 8. In some embodiments of the mixtures above, each chemokine or antagonist variant thereof consists of the specified sequence.

In some embodiments, the first chemokine consists of SEQ ID NO: 12, the second chemokine consists of SEQ ID NO: 10 and the third chemokine consists of SEQ ID NO: 14. In some embodiments, the first chemokine consists of SEQ ID NO: 5, the second chemokine consists of SEQ ID NO: 10 and the third chemokine consists of SEQ ID NO: 8.

### Polynucleotide Mixtures

The above mixtures of chemokines, or agonist or antagonist variants thereof, can be encoded by nucleic acid sequences comprised in polynucleotides. There, the present invention also provides polynucleotide mixtures comprising first and second, and optionally third, polynucleotides which encode any of the above combinations of first and second, and optionally third, chemokines, and agonist or antagonist variants thereof shown in Table 3 and described above. The polynucleotides may be DNA, cDNA or RNA, and, in some embodiments, it is preferred that the polynucleotides are cDNA. In some embodiments, each polynucleotide may also encode a signal sequence required for correct processing of each translated protein into the mature, functional form inside cells, such as human cells. If the protein is produced in bacteria, for example, it would not require the signal sequence, and the mature protein can be produced in that expression system. For example, the amino acid sequence of each of CCL5, CCL17 and CCL20, including the signal sequence thereof, is shown in SEQ ID NOs: 1, 4 and 7, respectively, and the nucleic acid sequence encoding these proteins are shown in SEQ ID NOs: 27-29, respectively. The amino acid sequences of the mature forms of CCL5, CCL17 and CCL20 are shown in SEQ ID NOs: 2, 5 and 8. However, the signal sequence used in the present invention does not have to be the wild-type signal sequence, and may be replaced with signal sequences from other secreted or membrane associated proteins, including from other species. Signal sequences are well-known, as discussed in Nielsen et al., 2019, and can be predicted using, for example, the program SIgnaIP (Bendtsen et al., 2004). Moreover, it is well-known in the art that signal sequences can be replaced with signal sequences from other proteins and/or other species, such as signal sequences from HSV gD or IgE.

Thus, the present invention also provides a polynucleotide mixture comprising a first polynucleotide and a second polynucleotide, wherein the first polynucleotide comprises a nucleic acid sequence which encodes a first chemokine, or an agonist or antagonist variant thereof, which is suitable for binding to CCR4 and the second polynucleotide comprises a nucleic acid sequence which encodes a second chemokine, or an agonist or antagonist variant thereof, which is suitable for binding to CCR5 and/or CCR6, wherein the polynucleotide mixture is suitable for modulating an immune response. In some embodiments, the first polypeptide encodes CCL17 or an agonist or antagonist variant thereof, and the second polynucleotide encodes CCL5 or CCL20, or an agonist or antagonist variant thereof, or SEQ ID NO: 20 or 26.

In some embodiments, the polynucleotide mixture comprising a first polynucleotide which comprises a nucleic acid sequence which encodes a first chemokine, or an agonist or antagonist variant thereof, which is suitable for binding to CCR4, a second polynucleotide which comprises a nucleic acid sequence which encodes a second chemokine, or agonist or antagonist variant thereof, which is suitable for binding to CCR5, and a third polynucleotide which comprises a nucleic acid sequence which encodes a third chemokine, or an agonist or antagonist variant thereof, which is suitable for binding to CCR20. In some embodiments, the first polypeptide encodes CCL17 or an agonist or antagonist variant thereof, and the second polynucleotide encodes CCL5, or an agonist or antagonist variant thereof, or SEQ ID NO: 20 or 26, and the third polynucleotide encodes CCL20 or an agonist or antagonist variant thereof.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6 or 13, and the second polynucleotide comprises a nucleic acid sequence which has at least 70%, 75%, 80%, 85%, 90%, 95%, 96%, 97%, 98%, 99% or 100% sequence identity to one of SEQ ID NO: 3, 9, 11, 15-18 and 21-24. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6 or 13, and the second polynucleotide comprises a nucleic acid sequence which has at least 70%, 75%, 80%, 85%, 90%, 95%, 96%, 97%, 98%, 99% or 100% sequence identity to one of SEQ ID NO: 3, 11, 16-18 and 21-24. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6 or 13, and the second polynucleotide comprises a nucleic acid sequence which has at least 70%, 75%, 80%, 85%, 90%, 95%, 96%, 97%, 98%, 99% or 100% sequence identity to one of SEQ ID NO: 9 or 15.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6 or 13, the second polynucleotide comprises a nucleic acid sequence which has at least 70%, 75%, 80%, 85%, 90%, 95%, 96%, 97%, 98%, 99% or 100% sequence identity to one of SEQ ID NO: 3, 11, 16-18 and 21-24, and the third polynucleotide comprises a nucleic acid sequence which has at least 70%, 75%, 80%, 85%, 90%, 95%, 96%, 97%, 98%, 99% or 100% sequence identity to SEQ ID NO: 9 or 15.

### Agonist Polynucleotide Mixtures

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, and the second polynucleotide comprises a nucleic acid sequence which has at least 70%, 75%, 80%, 85%, 90%, 95%, 96%, 97%, 98%, 99% or 100% sequence identity to SEQ ID NO: 3. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, and the second polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, and the second polynucleotide comprises a nucleic acid sequence which has at least 70%, 75%, 80%, 85%, 90%, 95%, 96%, 97%, 98%, 99% or 100% sequence identity to one of SEQ ID NOs: 21-24.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to one of SEQ ID NOs: 3 and 21-24, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to one of SEQ ID NO: 3, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to one of SEQ ID NOs: 21-24, and the third polynucleotide comprises a nucleic acid sequence which has at least 70%, 75%, 80%, 85%, 90%, 95%, 96%, 97%, 98%, 99% or 100% sequence identity to SEQ ID NO: 9.

In some embodiments, the first polynucleotide comprises a nucleic acid sequence which comprises SEQ ID NO: 6, the second polynucleotide comprises a nucleic acid sequence which has comprises SEQ ID NO: 3, and the third polynucleotide comprises a nucleic acid sequence which comprises SEQ ID NO: 9. In some embodiments, the first polynucleotide comprises a nucleic acid sequence which consists of SEQ ID NO: 6, the second polynucleotide comprises a nucleic acid sequence which has consists of SEQ ID NO: 3, and the third polynucleotide comprises a nucleic acid sequence which consists of SEQ ID NO:9.

### Antagonist Polynucleotide Mixtures

In some embodiments, the first polynucleotide encodes Met-CCL17, the second polynucleotide encodes Met-CCL20, Met-CCL5 or SEQ ID NO: 20. In some embodiments, the first polynucleotide encodes CCL17, and the second polynucleotide encodes Met-CCL5 or SEQ ID NO: 20. In some embodiments, the first polynucleotide encodes Met-CCL17, the second polynucleotide encodes Met-CCL5 or SEQ ID NO: 20 and the third polynucleotide encodes Met-CCL20.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, and the second polynucleotide comprises a nucleic acid sequence which has at least 70%, 75%, 80%, 85%, 90%, 95%, 96%, 97%, 98%, 99% or 100% sequence identity to any one of SEQ ID NO: 11 and 15-18. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, and the second polynucleotide comprises a nucleic acid sequence which has at least 70%, 75%, 80%, 85%, 90%, 95%, 96%, 97%, 98%, 99% or 100% sequence identity to any one of SEQ ID NO: 11 and 16-18. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, and the second polynucleotide comprises a nucleic acid sequence which has at least 70%, 75%, 80%, 85%, 90%, 95%, 96%, 97%, 98%, 99% or 100% sequence identity to SEQ ID NO: 11. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, and the second polynucleotide comprises a nucleic acid sequence which has at least 70%, 75%, 80%, 85%, 90%, 95%, 96%, 97%, 98%, 99% or 100% sequence identity to any one of SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, and the second polynucleotide comprises a nucleic acid sequence which has at least 70%, 75%, 80%, 85%, 90%, 95%, 96%, 97%, 98%, 99% or 100% sequence identity to SEQ ID NO: 17.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, and the second polynucleotide comprises a nucleic acid sequence which has at least 70%, 75%, 80%, 85%, 90%, 95%, 96%, 97%, 98%, 99% or 100% sequence identity to any one of SEQ ID NO: 11 and 16-18. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, and the second polynucleotide comprises a nucleic acid sequence which has at least 70%, 75%, 80%, 85%, 90%, 95%, 96%, 97%, 98%, 99% or 100% sequence identity to SEQ ID NO: 13 or 17.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to SEQ ID NO: 11, and the third polynucleotide comprises a nucleic acid sequence which has at least 70%, 75%, 80%, 85%, 90%, 95%, 96%, 97%, 98%, 99% or 100% sequence identity to SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to any one of SEQ ID NOs: 16-18, and the third polynucleotide comprises a nucleic acid sequence which has at least 70%, 75%, 80%, 85%, 90%, 95%, 96%, 97%, 98%, 99% or 100% sequence identity to SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to SEQ ID NOs: 17, and the third polynucleotide comprises a nucleic acid sequence which has at least 70%, 75%, 80%, 85%, 90%, 95%, 96%, 97%, 98%, 99% or 100% sequence identity to SEQ ID NO: 15.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to SEQ ID NO: 11, and the third polynucleotide comprises a nucleic acid sequence which has at least 70%, 75%, 80%, 85%, 90%, 95%, 96%, 97%, 98%, 99% or 100% sequence identity to SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to SEQ ID NO: 11, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to SEQ ID NO: 11, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to any one of SEQ ID NOs: 16-18, and the third polynucleotide comprises a nucleic acid sequence which has at least 70%, 75%, 80%, 85%, 90%, 95%, 96%, 97%, 98%, 99% or 100% sequence identity to SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to SEQ ID NO: 17, and the third polynucleotide comprises a nucleic acid sequence which has at least 70%, 75%, 80%, 85%, 90%, 95%, 96%, 97%, 98%, 99% or 100% sequence identity to SEQ ID NO: 15. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to any one of SEQ ID NOs: 16-18, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 13, the second polynucleotide comprises a nucleic acid sequence which has at least 70%, 75%, 80%, 85%, 90%, 95%, 96%, 97%, 98%, 99% or 100% sequence identity to SEQ ID NOs: 17, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to any one of SEQ ID NOs: 16-18, and the third polynucleotide comprises a nucleic acid sequence which has at least 70%, 75%, 80%, 85%, 90%, 95%, 96%, 97%, 98%, 99% or 100% sequence identity to SEQ ID NO: 9. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 6, the second polynucleotide comprises a nucleic acid sequence which has at least 70%, 75%, 80%, 85%, 90%, 95%, 96%, 97%, 98%, 99% or 100% sequence identity to SEQ ID NOs: 17, and the third polynucleotide comprises a nucleic acid sequence which has at least 70%, 75%, 80%, 85%, 90%, 95%, 96%, 97%, 98%, 99% or 100% sequence identity to SEQ ID NO: 9.

In some embodiments, the first polynucleotide comprises a nucleic acid sequence which consists of SEQ ID NO: 13 and the second polynucleotide comprises a nucleic acid sequence which consists of any one of SEQ ID NO: 11 and 15-18. In some embodiments, the first polynucleotide comprises a nucleic acid sequence which consists of SEQ ID NO: 13 and the second polynucleotide comprises a nucleic acid sequence which consists of any one of SEQ ID NOs: 16-18, preferably SEQ ID NO: 17. In some embodiments, the first polynucleotide comprises a nucleic acid sequence which consists of SEQ ID NO: 13 and the second polynucleotide comprises a nucleic acid sequence which consists of SEQ ID NO: 15.

In some embodiments, the first polynucleotide comprises a nucleic acid sequence which consists of SEQ ID NO: 6 and the second polynucleotide comprises a nucleic acid sequence which consists of any one of SEQ ID NO: 11 and 16-18. In some embodiments, the first polynucleotide comprises a nucleic acid sequence which consists of SEQ ID NO: 6 and the second polynucleotide comprises a nucleic acid sequence which consists of SEQ ID NO: 11 or 17.

In some embodiments, the first polynucleotide comprises a nucleic acid sequence which consists of SEQ ID NO: 13, the second polynucleotide comprises a nucleic acid sequence which consists of SEQ ID NO: 11, and the third polynucleotide comprises a nucleic acid sequence which consists of SEQ ID NO: 15. In some embodiments, the first polynucleotide comprises a nucleic acid sequence which consists of SEQ ID NO: 13, the second polynucleotide comprises a nucleic acid sequence which consists of any one of SEQ ID NOs: 16-18, preferably SEQ ID NO: 17, and the third polynucleotide comprises a nucleic acid sequence which consists of SEQ ID NO: 15.

In some embodiments, the first polynucleotide comprises a nucleic acid sequence which consists of SEQ ID NO: 6, the second polynucleotide comprises a nucleic acid sequence which consists of SEQ ID NO: 11, and the third polynucleotide comprises a nucleic acid sequence which consists of SEQ ID NO: 15. In some embodiments, the first polynucleotide comprises a nucleic acid sequence which consists of SEQ ID NO: 6, the second polynucleotide comprises a nucleic acid sequence which consists of any one of SEQ ID NOs: 16-18 preferably SEQ ID NO: 17, and the third polynucleotide comprises a nucleic acid sequence which consists of SEQ ID NO: 15. In some embodiments, the first polynucleotide comprises a nucleic acid sequence which consists of SEQ ID NO: 13, the second polynucleotide comprises a nucleic acid sequence which consists of SEQ ID NO: 11, and the third polynucleotide comprises a nucleic acid sequence which consists of SEQ ID NO: 9. In some embodiments, the first polynucleotide comprises a nucleic acid sequence which consists of SEQ ID NO: 13, the second polynucleotide comprises a nucleic acid sequence which consists of any one of SEQ ID NO: 16-18, preferably SEQ ID NO: 17, and the third polynucleotide comprises a nucleic acid sequence which consists of SEQ ID NO: 9. In some embodiments, the first polynucleotide comprises a nucleic acid sequence which consists of SEQ ID NO: 6, the second polynucleotide comprises a nucleic acid sequence which consists of SEQ ID NO: 11, and the third polynucleotide comprises a nucleic acid sequence which consists of SEQ ID NO: 9. In some embodiments, the first polynucleotide comprises a nucleic acid sequence which consists of SEQ ID NO: 6, the second polynucleotide comprises a nucleic acid sequence which consists of any one of SEQ ID NO: 16-18, preferably SEQ ID NO: 17, and the third polynucleotide comprises a nucleic acid sequence which consists of SEQ ID NO: 9.

### Polypeptide+Chemokine Compositions

In some embodiments, there are provided compositions comprising at least one polynucleotide and at least one chemokine, or agonist or antagonist variant thereof, wherein the composition corresponds to the above described chemokine mixtures and polynucleotide mixtures. Thus, there is provided a composition comprising a first polynucleotide comprising a nucleic acid sequence which encodes a first chemokine or an agonist or antagonist variant thereof which is suitable for binding to one of CCR5, CCR4 and/or CCR6, and comprising a second chemokine or an agonist or antagonist variant thereof which is suitable for binding to a different one of CCR5, CCR4 and/or CCR6 from the first chemokine or agonist or antagonist thereof, wherein the composition is suitable for modulating an immune response. The first polynucleotide can be any polynucleotide described above, and the second chemokine, or agonist or antagonist variant thereof, can be any chemokine, or agonist or antagonist variant thereof, described above. The composition may also comprise a second polynucleotide which comprises a nucleic acid sequence which encodes a third chemokine, or agonist or antagonist variant thereof, or the composition may comprise a third chemokine, or agonist or antagonist variant thereof, wherein the third chemokine, or agonist or antagonist variant thereof, is suitable for binding to a different one of CCR5, CCR4 and/or CCR6 from each of the first and second chemokines, or agonist or antagonist variants thereof. The second polynucleotide may be any polynucleotide described above, and the third chemokine, or agonist or antagonist variant thereof, may be any chemokine, or agonist or antagonist variant thereof, described above.

Thus, in some embodiments, the composition comprises first and a second polynucleotides, each encoding first and second chemokines, or agonist or antagonist variants thereof, respectively, and comprises a chemokine, or agonist or antagonist variant thereof. In other embodiments, the composition comprises a first polynucleotide encoding a first chemokine, or agonist or antagonist variant thereof, and comprises a second and third chemokine, or agonist or antagonist variant thereof.

In some embodiments, the composition comprises a first polynucleotide encoding a first chemokine, or agonist or antagonist variant thereof, and a second chemokine, or agonist or antagonist variant thereof, according to any combinations shown in Table 4. The first polynucleotide corresponds to any of the relevant polynucleotides described above, and the second chemokine, or agonist or antagonist variant thereof, corresponds to any of the relevant chemokines, or agonist or antagonist variants thereof, described above.

TABLE 4

| First polynucleotide, encoding | |
|---|---|
| a first chemokine or agonist | Second chemokine, or agonist |
| or antagonist variant thereof | or antagonist variant thereof |
| CCL17 | CCL5 |
| CCL17 | CCL20 |
| CCL17 | SEQ ID NO: 26 |
| CCL17 | Met-CCL5 |
| CCL17 | SEQ IDNO: 20 |
| Met-CCL17 | Met-CCL5 |
| Met-CCL17 | Met-CCL20 |
| Met-CCL17 | SEQ ID NO: 20 |
| CCL20 | CCL5 |
| CCL20 | Met-CCL5 |
| Met-CCL20 | Met-CCL5 |
| CCL20 | SEQ ID NO: 20 |
| CCL20 | SEQ ID NO: 26 |
| CCL5 | CCL17 |
| CCL5 | CCL20 |
| Met-CCL5 | Met-CCL17 |
| Met-CCL5 | CCL17 |
| Met-CCL5 | Met-CCL20 |
| Met-CCL5 | CCL20 |
| SEQ ID NO: 20 | Met-CCL17 |
| SEQ ID NO: 20 | CCL17 |
| SEQ ID NO: 20 | Met-CCL20 |
| SEQ ID NO: 20 | CCL20 |
| SEQ ID NO: 26 | CCL17 |
| SEQ ID NO: 26 | CCL20 |

In some embodiments, the composition comprises a first polynucleotide encoding a first chemokine, or an agonist or antagonist variant thereof; a second chemokine, or agonist or antagonist variant thereof; and comprises a second polynucleotide, encoding a third chemokine, or agonist or antagonist variant thereof, or comprises a third chemokine, or agonist or antagonist variant thereof, according to any of the combinations shown in Table 5 (where column three is labelled as “third chemokine, or agonist or antagonist variant thereof”, this includes the chemokine encoded by the second polynucleotide). The first and second polynucleotides independently correspond to any of the polynucleotides described above, and each of the chemokines, or agonist or antagonist variants thereof, independently corresponds to any of the chemokines, or agonist or antagonist variants, described above.

TABLE 5

| First polynucleotide, | | |
|---|---|---|
| encoding a first | Second chemokine, or | Third chemokine, or |
| chemokine or agonist or | agonist or antagonist | agonist or antagonist |
| antagonist thereof variant | variant thereof | variant thereof |
| CCL17 | CCL5 | CCL20 |
| CCL17 | CCL20 | CCL5 |
| CCL17 | SEQ ID NO: 26 | CCL20 |
| CCL17 | CCL20 | SEQ ID NO: 26 |
| CCL17 | Met-CCL5 | CCL20 |
| CCL17 | CCL20 | Met-CCL5 |
| CCL17 | SEQ ID NO: 20 | CCL20 |
| CCL17 | CCL20 | SEQ ID NO: 20 |
| Met-CCL17 | Met-CCL5 | Met-CCL20 |
| Met-CCL17 | Met-CCL20 | Met-CCL5 |
| Met-CCL17 | Met-CCL20 | SEQ ID NO: 20 |
| Met-CCL17 | SEQ ID NO: 20 | Met-CCL20 |
| CCL20 | CCL5 | CCL17 |
| CCL20 | CCL17 | CCL5 |
| CCL20 | Met-CCL5 | CCL17 |
| CCL20 | CCL17 | Met-CCL5 |
| Met-CCL20 | Met-CCL5 | Met-CCL17 |
| Met-CCL20 | Met-CCL17 | Met-CCL5 |
| CCL20 | SEQ ID NO: 20 | CCL17 |
| CCL20 | CCL17 | SEQ ID NO: 20 |
| Met-CCL20 | SEQ ID NO: 20 | Met-CCL17 |
| Met-CCL20 | Met-CCL17 | SEQ ID NO: 20 |
| CCL20 | SEQ ID NO: 26 | CCL17 |
| CCL20 | CCL17 | SEQ ID NO: 26 |
| CCL5 | CCL17 | CCL20 |
| CCL5 | CCL20 | CCL17 |
| Met-CCL5 | CCL17 | CCL20 |
| Met-CCL5 | CCL20 | CCL17 |
| Met-CCL5 | Met-CCL17 | Met-CCL20 |
| Met-CCL5 | Met-CCL20 | Met-CCL17 |
| SEQ ID NO: 20 | CCL17 | CCL20 |
| SEQ ID NO: 20 | CCL20 | CCL17 |
| SEQ ID NO: 20 | Met-CCL17 | Met-CCL20 |
| SEQ ID NO: 20 | Met-CCL20 | Met-CCL17 |
| SEQ ID NO: 26 | CCL17 | CCL20 |
| SEQ ID NO: 26 | CCL20 | CCL17 |

In some embodiments, the composition comprises a first polynucleotide encoding a first chemokine, or agonist or antagonist variant thereof; a second chemokine, or agonist or antagonist variant thereof; and a third chemokine, or agonist or antagonist variant thereof. In some embodiments, the first polynucleotide comprises a nucleic acid sequence which has at least 70%, 75%, 80%, 85%, 90%, 95%, 96%, 97%, 98%, 99% or 100% sequence identity to one of SEQ ID NOs: 3, 11, 16-18 and 21-24; the second chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NOs: 5 or 12; and the third chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NO: 8 or 14.

### Agonist Compositions

In some embodiments, the composition comprises a first polynucleotide encoding a first chemokine, or agonist variant thereof; a second chemokine, or agonist or thereof; and a third chemokine, or agonist variant thereof. In some embodiments, the first polynucleotide comprises a nucleic acid sequence which has at least 70%, 75%, 80%, 85%, 90%, 95%, 96%, 97%, 98%, 99% or 100% sequence identity to any one of SEQ ID NOs: 21-24; the second chemokine, or agonist variant thereof, comprises an amino acid sequence which has at least 70%, 75%, 80%, 85%, 90%, 95%, 96%, 97%, 98%, 99% or 100% sequence identity to SEQ ID NO: 5; and the third chemokine, or agonist variant thereof, comprises an amino acid sequence which has at least 70%, 75%, 80%, 85%, 90%, 95%, 96%, 97%, 98%, 99% or 100% sequence identity to SEQ ID NO: 8.

In some embodiments, the composition comprises a first polynucleotide encoding a first chemokine, or agonist variant thereof, wherein the first polynucleotide comprises a nucleic acid sequence comprising any one of SEQ ID NOs: 21-24; a second chemokine, or agonist variant thereof, comprises the amino acid sequence of SEQ ID NO: 5, and a third chemokine, or agonist variant thereof, comprising the amino acid sequence of SEQ ID NO: 8.

In some embodiments, the composition comprises a first polynucleotide encoding a first chemokine, or agonist variant thereof, wherein the first polynucleotide comprises a nucleic acid sequence consisting of any one of SEQ ID NOs: 21-24; a second chemokine, or agonist variant thereof, which consists of SEQ ID NO: 5, and a third chemokine, or agonist variant thereof, which consists of SEQ ID NO: 8.

### Antagonist Composition

In some embodiments, the composition comprises a first polynucleotide encoding a first chemokine, or antagonist variant thereof; a second chemokine, or antagonist variant thereof; and a third chemokine, or antagonist variant thereof. In some embodiments, the first polynucleotide comprises a nucleic acid sequence which has at least 70%, 75%, 80%, 85%, 90%, 95%, 96%, 97%, 98%, 99% or 100% sequence identity to any one of SEQ ID NOs: 16-18; the second chemokine, or antagonist variant thereof, comprises an amino acid sequence which has at least 70%, 75%, 80%, 85%, 90%, 95%, 96%, 97%, 98%, 99% or 100% sequence identity to SEQ ID NO: 12; and the third chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NO: 14. In some embodiments, the first polynucleotide comprises a nucleic acid sequence which has at least 70%, 75%, 80%, 85%, 90%, 95%, 96%, 97%, 98%, 99% or 100% sequence identity to any one of SEQ ID NOs: 16-18; the second chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NOs: 5; and the third chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NO: 8.

In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 17; a second chemokine, or antagonist variant thereof, which comprises an amino acid sequence which has at least 70%, 75%, 80%, 85%, 90%, 95%, 96%, 97%, 98%, 99% or 100% sequence identity to SEQ ID NO: 12; and a third chemokine, or agonist or antagonist variant thereof, which comprises an amino acid sequence which has at least 70%, 75%, 80%, 85%, 90%, 95%, 96%, 97%, 98%, 99% or 100% sequence identity to SEQ ID NO: 14. In some embodiments, the first polynucleotide comprises a nucleic acid sequence which has at least 70%, 75%, 80%, 85%, 90%, 95%, 96%, 97%, 98%, 99% or 100% sequence identity to SEQ ID NO: 17; the second chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NOs: 5; and the third chemokine, or agonist or antagonist variant thereof, comprises an amino acid sequence which has at least 70%, 75%, 80%, 85%, 90%, 95%, 96%, 97%, 98%, 99% or 100% sequence identity to SEQ ID NO: 8.

In some embodiments, the composition comprises a first polynucleotide encoding a first chemokine, or antagonist variant thereof, wherein the first polynucleotide comprises a nucleic acid sequence comprising any one of SEQ ID NOs: 16-18; a second chemokine, or antagonist variant thereof, comprises the amino acid sequence of SEQ ID NO: 12, and a third chemokine, or agonist variant thereof, comprising the amino acid sequence of SEQ ID NO: 14. In some embodiments, the composition comprises a first polynucleotide encoding a first chemokine, or antagonist variant thereof, wherein the first polynucleotide comprises a nucleic acid sequence comprising any one of SEQ ID NOs: 16-18; a second chemokine, or antagonist variant thereof, comprises the amino acid sequence of SEQ ID NO: 5, and a third chemokine, or agonist variant thereof, comprising the amino acid sequence of SEQ ID NO: 8. In some embodiments, the composition comprises a first polynucleotide encoding a first chemokine, or antagonist variant thereof, wherein the first polynucleotide comprises a nucleic acid sequence comprising SEQ ID NO: 17; a second chemokine, or antagonist variant thereof, comprises the amino acid sequence of SEQ ID NO: 12, and a third chemokine, or agonist variant thereof, comprising the amino acid sequence of SEQ ID NO: 14. In some embodiments, the composition comprises a first polynucleotide encoding a first chemokine, or antagonist variant thereof, wherein the first polynucleotide comprises a nucleic acid sequence comprising SEQ ID NO: 17; a second chemokine, or antagonist variant thereof, comprises the amino acid sequence of SEQ ID NO: 5, and a third chemokine, or agonist variant thereof, comprising the amino acid sequence of SEQ ID NO: 8.

In some embodiments, the composition comprises a first polynucleotide encoding a first chemokine, or antagonist variant thereof, wherein the first polynucleotide comprises a nucleic acid sequence consisting of any one of SEQ ID NOs: 16-18; a second chemokine, or antagonist variant thereof, consisting of SEQ ID NO: 12, and a third chemokine, or agonist variant thereof, consisting of SEQ ID NO: 14. In some embodiments, the composition comprises a first polynucleotide encoding a first chemokine, or antagonist variant thereof, wherein the first polynucleotide comprises a nucleic acid sequence consisting of SEQ ID NOs: 16-18; a second chemokine, or antagonist variant thereof, consisting of SEQ ID NO: 5, and a third chemokine, or agonist variant thereof, consisting of SEQ ID NO: 8. In some embodiments, the composition comprises a first polynucleotide encoding a first chemokine, or antagonist variant thereof, wherein the first polynucleotide comprises a nucleic acid sequence consisting of SEQ ID NO: 17; a second chemokine, or antagonist variant thereof, consisting of SEQ ID NO: 12, and a third chemokine, or agonist variant thereof, consisting of SEQ ID NO: 14. In some embodiments, the composition comprises a first polynucleotide encoding a first chemokine, or antagonist variant thereof, wherein the first polynucleotide comprises a nucleic acid sequence consisting of SEQ ID NO: 17; a second chemokine, or antagonist variant thereof, consisting the amino acid sequence of SEQ ID NO: 5, and a third chemokine, or agonist variant thereof, consisting of the amino acid sequence of SEQ ID NO: 8.

### Nucleic Acid Construct Mixture

Also provided herein are nucleic acid construct mixtures corresponding to the above described polynucleotide mixtures and compositions. In particular, the nucleic acid construct mixtures of the present invention comprise nucleic acid constructs which correspond to the polynucleotides in each of the polynucleotide mixtures and compositions of the present invention. For example, where a polynucleotide mixture comprises first and second polynucleotides, the nucleic acid construct mixture comprises a first nucleic acid construct which comprises the first polynucleotide, and a second nucleic acid construct which comprises the second polynucleotide. Where a polynucleotide mixture also comprises a third polynucleotide, the nucleic acid construct mixture comprises a third nucleic acid construct which comprises the third polynucleotide. Similarly, where a composition comprises first and second polynucleotides, the nucleic acid construct mixture comprises a first nucleic acid construct which comprises the first polynucleotide, and a second nucleic acid construct which comprises the second polynucleotide.

### Nucleic Acid Construct

Also provided herein are nucleic acid constructs, which each encode all of the polynucleotides in a polynucleotide mixture or composition of the invention. Thus, where a polynucleotide mixture of the invention comprises first and second polynucleotides, the nucleic acid construct comprises both the first and second polynucleotides. Where a polynucleotide mixture of the invention comprises first, second and third polynucleotides, the nucleic acid construct comprises the first, second and third polynucleotides. Where a composition of the invention comprises a first polynucleotide, the nucleic acid construct comprises the first polynucleotide. Where a composition of the invention comprises first and second polynucleotides, the nucleic acid construct comprises the first and second polynucleotides.

Each of the nucleic acid constructs described above, including in the nucleic acid construct mixture, may comprises further elements required for expression of the polynucleotide(s) comprised therein. For example, each nucleic acid construct may comprise a promoter for initiation of expression, which is operably linked to the polynucleotide, and/or a polyadenylation site for termination of expression. Where a nucleic acid construct comprises more than one polynucleotide, there may be a promoter operably linked to each of the polynucleotides, or there may be a single promoter which is operably linked in such a way as to enable expression of all of the polynucleotides.

### Host Cell

In some embodiments, there is provided a host cell which comprises the polynucleotide mixture, nucleic acid construct mixture or the nucleic acid construct described above. The host cell may be prokaryotic or eukaryotic, and may include bacterial cells, fungal cells such as yeast, plant cells, insect cells, or mammalian cells. In a preferred embodiment, the mammalian cell is a human cell. Preferably, the host cell expresses the polynucleotide(s) comprised therein.

### Pharmaceutical Composition

In some embodiments, there is provided a pharmaceutical composition which comprises the chemokine mixture, polynucleotide mixture, composition, nucleic acid construct mixture, nucleic acid construct, or host cell of the present invention. The pharmaceutical composition further comprises a pharmaceutically-acceptable carrier, excipient or diluent. Suitable pharmaceutical carriers are described in “Remington's Pharmaceutical Sciences” by E. W. Martin, 1995. Examples of pharmaceutical compositions include any solid (tablets, pills, capsules, granules, etc.) or liquid (solutions, suspensions, emulsions, etc.) compositions for oral, topical or parenteral administration.

In some embodiments, the pharmaceutical composition comprises further a therapeutic or prophylactic, and/or an adjuvant. The adjuvant may be any adjuvant known in the art, for example, GM-CSF or G-CSF.

### Use

The chemokine mixture, polynucleotide mixture, composition, nucleic acid construct mixture, nucleic acid construct, host cell and pharmaceutical composition of the invention are for use as an immunostimulant or an immunosuppressant. In particular, the chemokine mixture, polynucleotide mixture, composition, nucleic acid construct mixture, nucleic acid construct, host cell and pharmaceutical composition of the invention targets regulatory T-cells, which modulate the immune response. In some embodiments, the chemokine mixture, polynucleotide mixture, composition, nucleic acid construct mixture, nucleic acid construct, host cell and pharmaceutical composition of the invention is for use in the treatment or prevention of a disease or disorder characterized by altered levels of CCR1, 4, 5, 6, and 8 or their binding chemokines, or a disease or disorder associated with a dysregulated immune response. In some embodiments, the treatment includes using the chemokine mixture, polynucleotide mixture, composition, nucleic acid construct mixture, nucleic acid construct, host cell and pharmaceutical composition as an adjuvant.

In some embodiments, the disease or disorder is cancer, a viral infection, Alzheimer's disease, an autoimmune disease, an inflammatory disease or condition, an allergy or a disease or condition associated with a dysregulated immune response.

The viral infection may be a HIV infection, HIV/AIDS, a HSV infection, for example, HSV1 or HSV2, influenza, a coronavirus infection, for example, a human coronavirus infection, such as SARS-CoV-2, or a SARS-like virus, human cytomegalovirus, Epstein-barr virus, a Rhinovirus infection or hepatitis virus B or C.

The cancer may be a solid tumour such as prostate cancer, breast cancer or lymphomas.

The autoimmune disease may be arthritis, such as rheumatoid arthritis, Crohn's disease or chronic obstructive pulmonary disease.

Dysregulated immune responses may include conditions considered to have features of innate responses such as Alzheimers disease or multiple sclerosis, or overactive responses to infections such as infectious mononucleosis. Diseases and conditions associated with a dysregulated immune responses also include diseases and conditions associated with a runaway immune response, for example, a cytokine storm, such as SARS-CoV-2. Diseases and conditions associated with a dysregulated immune response may also include rheumatoid arthritis or CNS or pulmonary inflammatory disease.

Allergies may include feline allergies, dust mite allergies and hayfever. In some embodiments, the chemokine mixture, polynucleotide mixture, composition, nucleic acid construct mixture, nucleic acid construct, host cell and pharmaceutical composition may be injected together with an allergen, such as feline hair allergens, in order to induce tolerance to the allergen or decrease the immune response to the allergen.

In some embodiments, the chemokine mixture, polynucleotide mixture, composition, nucleic acid construct mixture, nucleic acid construct, host cell and pharmaceutical composition of the invention is used as an immunosuppressant, to reduce or prevent an immune response. In these embodiments, the chemokine mixture, polynucleotide mixture or composition targets activated regulatory T-cells, which then downregulate the immune response, such as a T-cell response or an antibody response. Thus, the agonist mixtures and compositions described above can be used as such an immunosuppressant, by targeting and recruiting activated T-cells. In these embodiments, the diseases and/or conditions to be treated are preferably those which are associated with an overactive or runaway immune response, or are autoimmune diseases, inflammatory diseases and allergies. Specific examples of these diseases and conditions are described above.

In some embodiments, the chemokine mixture, polynucleotide mixture, composition, nucleic acid construct mixture, nucleic acid construct, host cell and pharmaceutical composition of the invention is used as an immunostimulant. In these embodiments, the chemokine mixture, polynucleotide mixture or composition targets regulatory T-cells, but blocks recruitment of activated regulatory T-cells. This may be either by antagonising CC chemokine receptors which are markers of activated regulatory T-cells, or by agonising CC chemokine receptors which are markers of regulatory T-cells (e.g. CCR4, CCR8 and CCR6) but antagonising CC chemokine receptors which are markers of activated T-cells (e.g. CCR5). In this latter embodiment, regulatory T-cells are recruited, but they are not activated, such that they do not suppress the immune response. This blocking of regulatory T-cells permits an immune response, such as a T-cell response or an antibody response. Thus, the antagonist mixtures and compositions described above can be used as an immunostimulant, by targeting and blocking regulatory T-cells. In these embodiments, the diseases and/or conditions to be treated are preferably cancer, a viral infection or Alzheimer's disease, and specific examples of these diseases and conditions are described above. These antagonist mixtures and compositions can also be used as adjuvants, thereby increasing the immune response induced by therapeutic or prophylactic treatments.

### Method of Treatment

In some embodiments, there is provided a method of treatment of a patient in need thereof, wherein the method comprises administering, to the patient, a chemokine mixture, polynucleotide mixture, composition, nucleic acid construct mixture, nucleic acid construct, host cell or pharmaceutical composition according to the present invention and described above. In some embodiments, the patient is a human or non-human animal, preferably a human. In some embodiments, the chemokine mixture, polynucleotide mixture, composition, nucleic acid construct mixture, nucleic acid construct, host cell and pharmaceutical composition is administered as a therapeutic or prophylactic treatment. In other embodiments, the chemokine mixture, polynucleotide mixture, composition, nucleic acid construct mixture, nucleic acid construct, host cell and pharmaceutical composition is administered as an adjuvant. The treatment may be of a disease or disorder characterized by altered levels of CCR1, 4, 5, 6, and 8 or their binding chemokines, or a disease or disorder associated with an aberrant immune response, as described above. Thus, in some embodiments, the chemokine mixture, polynucleotide mixture, composition, nucleic acid construct mixture, nucleic acid construct, host cell or pharmaceutical composition agonises regulatory T-cells, to suppress or prevent an immune response. In other embodiments, the chemokine mixture, polynucleotide mixture, composition, nucleic acid construct mixture, nucleic acid construct, host cell or pharmaceutical composition antagonises regulatory T-cells, to permit or increase an immune response.

### Administration

The chemokine mixtures, polynucleotide mixtures, compositions, nucleic acid construct mixture, nucleic acid constructs, host cells and pharmaceutical compositions of the invention may be administered by any suitable method known to those skilled in the art. For example, administration may be accomplished orally or parenterally. Methods of parenteral delivery include topical, intra-arterial, intramuscular, subcutaneous, intramedullary, intrathecal, intraventricular, intravenous, intraperitoneal, mucosal or intranasal administration. In some embodiments, for example, the treatment of arthritis, the chemokine mixture, polynucleotide mixture, composition, nucleic acid construct mixture, nucleic acid construct, host cell and pharmaceutical composition may be injected directly into the joint to be treated. In some embodiments, where the chemokine mixture, polynucleotide mixture, composition, nucleic acid construct mixture, nucleic acid construct, host cell and pharmaceutical composition is for the treatment or prevention of allergies, the chemokine mixture, polynucleotide mixture, composition, nucleic acid construct mixture, nucleic acid construct, host cell and pharmaceutical composition may be injected subcutaneously, in the style of an epi-pen. In embodiments where the chemokine mixture, polynucleotide mixture, composition, nucleic acid construct mixture, nucleic acid construct, host cell and pharmaceutical composition is for the treatment of a disease or condition of a respiratory disease associated with a cytokine storm (e.g. SARS-CoV-2 and influenza), the chemokine mixture, polynucleotide mixture, composition, nucleic acid construct mixture, nucleic acid construct, host cell and pharmaceutical composition may be administered to the lungs by means of an inhaler.

Where the chemokine mixture, polynucleotide mixture, composition, nucleic acid construct mixture, nucleic acid construct, host cell and pharmaceutical composition of the invention is used as a prophylactic treatment, it may be administered at least once, at least twice or at least three times, with each administration being separated by at least a week, at least two weeks or at least three weeks. Preferably, the chemokine mixture, polynucleotide mixture, composition, nucleic acid construct mixture, nucleic acid construct, host cell or pharmaceutical composition is administered twice, with each administration separated by three weeks.

Where the chemokine mixture, polynucleotide mixture, composition, nucleic acid construct mixture, nucleic acid construct, host cell and pharmaceutical composition is used as a therapeutic treatment, it may be administered once every week, once every two weeks, once every three weeks, or once a month. For example, the chemokine mixture, polynucleotide mixture, composition, nucleic acid construct mixture, nucleic acid construct, host cell and pharmaceutical composition may be administered upon occurrence or diagnosis of the disease or condition and, thereafter, may be administered on a maintenance schedule every two weeks.

### Use in the Manufacture of a Medicament

In some embodiments, the chemokine mixture, polynucleotide mixture, composition, nucleic acid construct mixture, nucleic acid construct, host cell or pharmaceutical composition of the present invention is used in the manufacture of a medicament. The medicament is for use in the treatment or prevention of a disease or disorder characterized by altered levels of CCR1, 4, 5, 6, and 8 or their binding chemokines, or a disease or disorder associated with an aberrant immune response, or is for use as an immunostimulant or an immunosuppressant, as described above.

### Kit

In some embodiments, there is provided a kit comprising a chemokine mixture, polynucleotide mixture, composition, nucleic acid construct mixture, nucleic acid construct, host cell or pharmaceutical composition of the present invention. The kit may be for use in treating or preventing a disease or disorder characterized by altered levels of CCR1, 4, 5, 6, and 8 or their binding chemokines, or a disease or disorder associated with an aberrant immune response, or is for use as an immunostimulant or an immunosuppressant, as described above.

In some embodiments, the kit comprises ready-made chemokine mixtures, polynucleotide mixtures, compositions, nucleic acid construct mixtures or pharmaceutical compositions of the invention. In other embodiments, the kit comprises separate chemokines, polynucleotides or nucleic acid constructs in separate containers. The mixtures and compositions of the invention are then prepared from the kit by mixing appropriate amounts of each chemokine, polynucleotide or nucleic acid construct together. The kit may also comprise a pharmaceutically-acceptable carrier, diluent or excipient, which can be combined with the mixtures or compositions in a suitable amount, to form a pharmaceutical composition.

### EXAMPLES

### Example 1

The human herpes viruses HHV-6A and HHV-6B can integrate in the telomeric region of the host genome, and are integrated in approximately 1% of human populations, in roughly equal proportions: 0.2% HHV-6A and 0.4% HHV-6B (Tweedy, 2016 #18). These appear to be ancient events, and have led to Mendellian inherited lineages, making these integrations appear as endogenous virus genomes. While the inherited human chromosomally integrated HHV-6B (iciHHV-6B) genomes appear to co-segregate with circulating virus HHV-6B, the inherited chromosomally integrated HHV-6A (iciHHV-6A) genomes appear distinct (Tweedy, 2015; Tweedy, 2016; Greninger, 2018). Our analyses by deep next generation sequencing, showed one of the oldest lineages related to an integration event in the telomere of chromosome 17p. Surprisingly, our analyses of the genome showed it had all the genes intact as well as known cis acting sequences required for virus replication. As described herein, the encoded immunomodulatory genes include unexpectedly one with a novel spliced product with distinct properties (as shown in FIG. 2).

In the circulating HHV-6A virus, the U83A gene encodes a chemokine-like molecule, which can mediate immune cell chemotaxis with a unique specificity via interaction with an array of human chemokine receptors (Catusse, 2009; Catusse, 2007; Clark, 2013; Dewin, 2006). This specificity was distinct from that of any other human chemokine or microbial peptide. We hypothesized, that if the integrated genome is from an ancestral virus infecting a homo sapien or hominid ancestor, then the immunomodulatory genes may be distinct. While we found that, in the immunomodulatory gene, chemokine U83A, the gene structure was maintained across the genome, we unexpectedly found that, in iciHHV-6A, iciU83A, (FIG. 1) the transcript is distinct (cDNA, FIG. 2) and encodes for altered protein products (FIG. 2).

The circulating virus U83A gene is spliced using non-canonical splice donor and acceptor sites contained within an unusual direct repeat sequence, CT-AC within TACC (French, 1999; Tweedy, 2015). In the iciHHV-6A genome, these cis acting elements are maintained, but there is a distinct non-synonymous SNP proximal to the provisional splice acceptor site, which affects the acceptor splicing sequence consensus site and as such was placed to disrupt the unusual splicing event (FIG. 1). Moreover, our earlier results had shown evidence for the full length unspliced transcript expressed in two individuals with heart disease and integrated iciHHV-6A (Tweedy, 2015). Therefore, it was not clear whether any splicing event at all occurred at this locus in iciHHV-6A. This is shown in our annotations for the first genome sequence for the iciHHV-6A, where only the full-length gene product is shown (NCBI NC_001664.4 reference genome sequence for HHV-6A and KT895199.1 for iciHHV-6A). Remarkably, although in the virus the full length U83A encodes the full signal sequence resulting in the mature secreted product, this is in fact only rarely expressed in circulating virus. This is due to variation in a poly-T tract which disrupts expression of the gene by giving a frame-shift mutation, so the signal sequence is no longer made and the product not secreted. However, in the iciHHV-6A genome, the iciU83A gene is genetically fixed to encode the full signal sequence with a fixed length poly-T tract (Tweedy et al., 2015). Furthermore, it was not clear whether any splicing occurred at this ancestral iciU83A gene. Transcriptomics analyses using RNAseq on circulating virus gene expression identified only an anti-sense spliced transcript mapping to the U83 loci or restricted gene expression in integrated iciHHV-6A/B genomes, consisting of immediate early genes involved in gene control and not further describing U83A (Peddu, 2019). An earlier review on virus chemokines noted restricted gene expression, and also noted lack of splicing U83A in transcripts identified in people harbouring iciHHV-6A genomes, citing our previous genomic analyses citing two iciHHV-6A integrated genomes with only full length transcript expression of U83A detected in two patient donors (Pontejo et al., 2018; Tweedy et al., 2015).

By investigating the individual gene expression in transfected cells in vitro, we have shown that the integrated U83A gene (iciU83A), can be expressed and spliced, as described herein. This was identified by using both genomic prediction and by cDNA analyses of ciU83A gene in expression vectors transfected into cells and then characterised using RT-PCR followed by sequence determination. This showed that, despite the previous in vivo and cellular characterisations of iciHHV-6A individuals, and despite the SNP proximal to the splice donor site, the non-consensus splicing can be utilised (FIG. 2). This mutation causes a coding mutation from that in the virus, from GAT(Asp) to GGT(Gly) (FIGS. 1 and 2) in the full-length human integrated iciU83A gene. In addition, we have surprisingly found that this same coding mutation, from the frameshift due to the splicing, precisely disrupts the spliced stop codon that had led to the truncated version of the virus U83A gene, U83A-N (as it only includes the encoded N-terminal half of the molecule) (FIG. 2).

In the virus genome, U83A and U83A-N encode, respectively, the full length and the truncated protein product from the spliced gene (Dewin et al., 2006). These function as paired agonist and antagonist versions of the U83A chemokine, and are central to orchestrating immune cell attraction to the virus replicative site for virus dissemination or immune evasion. Surprisingly, in the human chromosomally integrated HHV-6A (iciU83A), the spliced product is no longer truncated at this site. Instead, it is now extended to a downstream stop codon and results in an extended truncated product iciU83A-N (FIG. 2). This now has a hydrophobic tag of 8 amino acids (FIG. 2). This can act to associate with the membrane or mediate multimers, both leading to stabilisation and altered presentation.

The spliced product has discrete functional domains. We have previously demonstrated that the encoded N-terminal domain dictates specificity of chemokine receptor interactions (Dewin et al., 2006). This could be delineated to a 17 amino acid peptide region, with the determinant of specificity between CCR2 and CCR5 interactions determined by a single arginine residue (Clark et al., 2013). In the iciU83A-N molecule this N-terminal domain remains intact (FIGS. 1 and 2), representing the receptor specificity as previously defined. In fact, in comparisons of the virus U83A-N spliced molecule to the full length U83A, all of the receptor specificity was maintained, with only the C-terminal signaling domain being abrogated in the spliced truncated molecule. This functional N-terminal domain is also preserved in the iciU83A-N molecule, with the encoded N-terminal binding domain being completely intact, but the C-terminal signaling domain being removed. However, surprisingly, the iciU83A-N molecule is not only truncated, it also has a C-terminal extension of the eight amino acid constituting a hydrophobic extension (FIG. 2). This is composed of an aromatic residue rich domain, which, via multiple tryptophan residues, can increase membrane association as well as unique multimerisation of the molecule. Such tryptophan ‘tagging’ experimentally has increased stability and multimerisation of covalently attached peptides as well as disrupting lipid membrane interactions, increasing bacteriocidal activities, and promoting multimerisation (Kamei et al., 2018; Singh et al., 2017; Yau et al., 1998). Therefore, this hydrophobic C-terminal tag in the iciU83A-N encoded molecule provides superior and distinct activities to other virus encoded chemokine molecules.

The specificity reported derived from the maintained N-terminal domain includes targeting CCR1, 4, 5, 6 and 8 receptors (Catusse et al., 2009; Catusse et al., 2007; Dewin et al., 2006). This unique combination allows targeting of immunesuppresive T-regulator lymphocytes, particularly via CCR4 and CCR6. The human CCR6 is monospecific for CCL20. Therefore, expanding receptor interactions including CCR6 is a unique property of the iciU83A-N molecule. The unique application of iciU83A-N is in the ability to act as an antagonist of these receptors. This is because the C-terminally signaling moiety is no longer present. Antagonism of CCR4 in particular has been demonstrated as a novel mechanism for increasing immunity to a target antigen (Bayry et al., 2008) with utility also in uncovering immune-reactivity to tumors by altering the tumour microenvironment (Vilgelm et al., 2019).

The iciU83A and iciU83A-N genes have some unusual cis acting features. We had shown previously that the virus U83A gene has a poly-T motif towards the N-terminal encoding region. This gives rise to instability of the 5′ end of the gene because variations in the number of the T bases can result in frame shift mutations which cause premature termination of the encoded peptide, thereby controlling expression of the U83A gene (FIG. 1) (Dewin et al., 2006; Tweedy et al., 2015). This appears to be controlled by a herpesvirus DNA editing mechanism, which promotes variation in a number of human herpesvirus. For example, in herpes simplex virus, human alpha herpesvirus 1 and 2, DNA editing occurs at poly C or G tracts, which can occur in this GC rich genome, these give rise to homopolymer frame shift mutations (HFM), resulting due to individual variations in the homopolymer tracts; in contrast, in HHV-6, which has an AT rich genome, HFM occur here in a poly T tract (Tweedy et al., 2016; Tweedy et al., 2017; Dewin et al., 2006; Tweedy et al., 2015). Only with an in frame set of poly T bases can the full gene be expressed which then encodes the N-terminal signal sequence required for co-translational insertion into the endoplasmic reticulum, followed by cleavage and processing for secretion of the mature U83A chemokine-like molecule. In the circulating virus, this is a rare variant, as most have disrupted U83A genes via the poly T tract, and evidence has been presented that this can vary during a single infection (Dewin et al., 2006; Tweedy et al., 2015). However, distinct from the virus, the integrated genome of iciHHV-6A, ciU83A genomes integrated at the 17p locus at the subtelomere/telomere region, only have the poly T tract that permits the full length ciU83A molecule to be produced. Deep sequencing shows this is the dominant or only form, unlike in circulating virus (Tweedy et al., 2015; Tweedy et al., 2015; Tweedy et al., 2016; Tweedy et al., 2017). This form, iciU83A, has only two non-synonymous SNPs, giving coding changes compared to circulating virus U83A (FIG. 2). One of these are in the N-terminal regions, but are part of natural variation in the exogenous viruses (Clark et al., 2013). The second is the mutation proximal to the splice acceptor site (FIG. 2). Since the N-terminal region is maintained compared to the virus U83A, this shows sharing of receptor specificities. Only the C-terminal signaling domain has this single non-synonymous mutation.

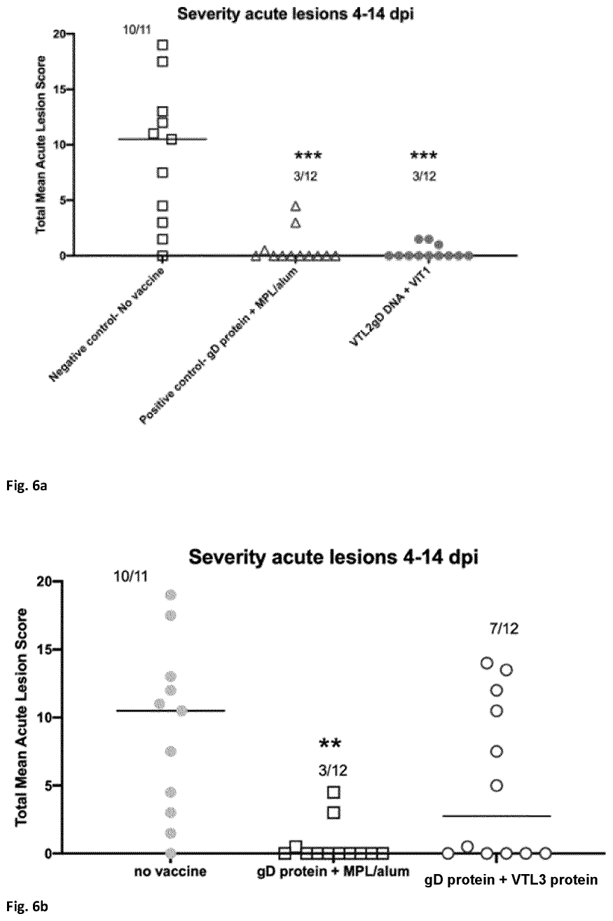

Analyses of the length of the poly T tract in strain variants and other integrated virus genomes shows that the full length gene can only be stabilized if the poly T tract is disrupted. Therefore, to fix the gene in the full-length encoded functional version, we mutated this poly T tract, while retaining the same codon usage and coding potential. We discovered that this fixes the gene in the functional version, encoding a signal sequence so that the mature product can be secreted (FIG. 1) and we introduced it here for function for iciU83A and iciU83A-N(SEQ ID NOs 17, 21 and 23; FIG. 6a).

U83A and iciU83A share two other novel features in their gene structures, which affect gene expression. First, both contain a direct repeat TACC, which is novel to this gene. In addition, the TACC motif forms part of a non-consensus splice donor and acceptor pair CT-AC, which we previously identified in the disrupted smaller gene product (French et al., 1999). The splice donor/acceptor pair in the circulating virus gene U83A, is however recognised by the cellular splicing apparatus, most likely via the minor spliceosome, since it is spliced when individually expressed in human cell lines (French et al., 1999; Lin et al., 2010). Although both these features are present in the iciU83A gene, the predicted splicing effects are completely different. The U83A gene characteristically is spliced to introduce a stop codon at the splice site, resulting in a truncated U83A-N product half the size of the full length product. However, this stop codon, TGA, is mutated in the iciU83A-N, to TGG (FIGS. 1 and 2) (while in frame with the full length iciU83A gene this give rise to the coding mutation Asp-Gly; FIG. 2) and, since this is proximal to the splice acceptor site, could also disrupt splicing.

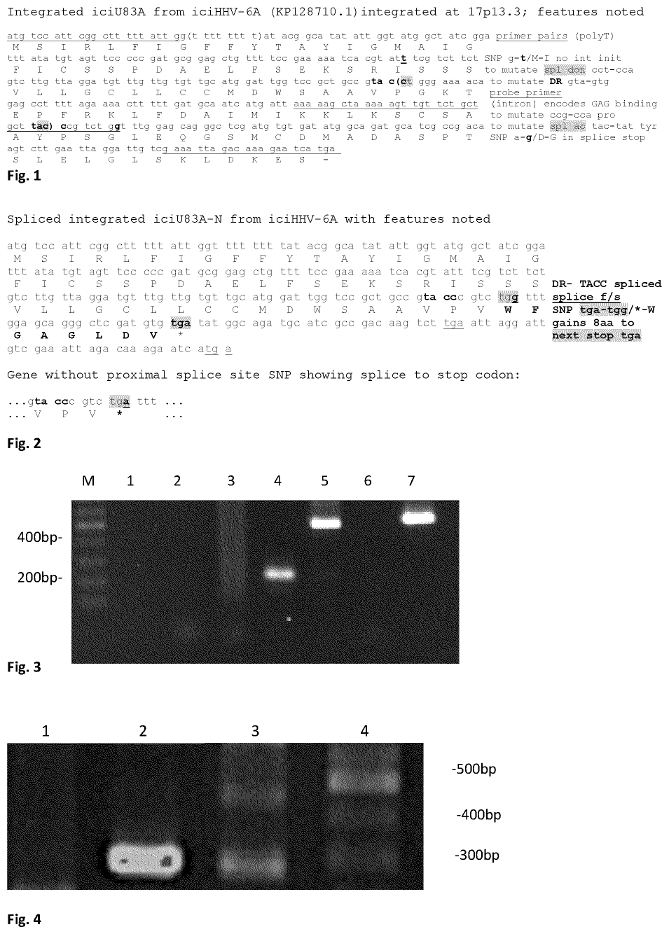

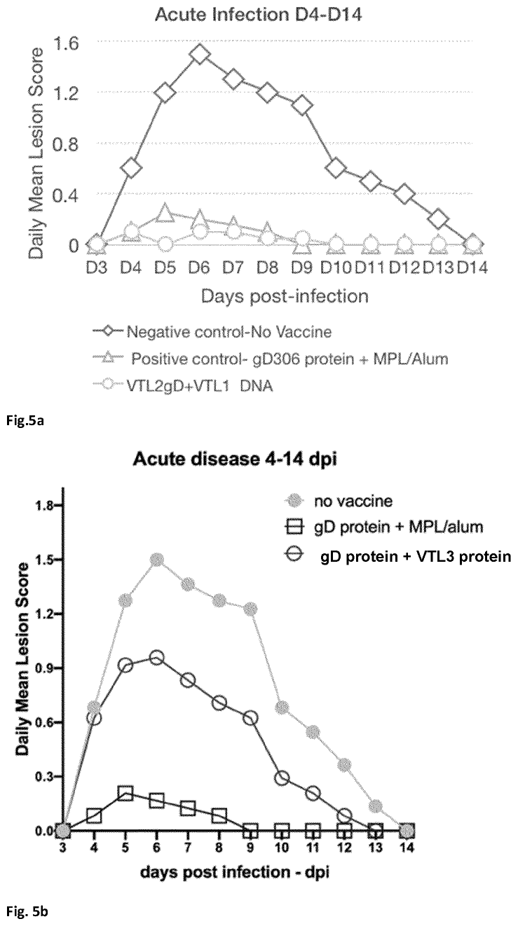

Using plasmid DNA expression vectors, containing a human cytomegalovirus IE gene promoter and SV40 virus polyadenylation site, we cloned this product and transduced into cell lines, HEK293, using transfection reagent. RNA was extracted then analysed by reverse transcription polymerase chain reaction (RT-PCR). The results showed that this splice donor site is utilised, and moreover that the spliced product, now reads through the site of the previous stop codon, and unusually extends the coding region by eight amino acids as described above (FIGS. 3 and 4).

In the circulating virus, the encoded full length U83A is rare, due to control by both the poly T tract disrupting the gene, as well as the non-consensus cellular splicing, truncating the full length product. Late in infection the splicing can be suppressed, resulting in read through of the full-length gene product (Dewin et al., 2006; Tweedy et al., 2015). In order to simulate this effect in the absence of controls exerted by the circulating virus gene expression, both the direct repeat and the splice donor/acceptor pair can be mutated. This has been done on the iciU83A gene while maintaining both codon usage and coding potential as shown herein. In SEQ ID Nos: 21-23, we have modified full-length iciU83A to stabilize expression for the uses described herein. SEQ ID NO: 21 removes N-terminal heterogeneity; SEQ ID NO: 22 prevents splicing; SEQ ID NO: 23 removes N-terminal heterogeneity and prevents splicing. Therefore, these products are now uniquely fixed, to remove heterogeneity (SEQ ID NO: 21 and 23), or to fix the full length version (SEQ ID NO: 22 and 23), for utility for immunostimulation as reviewed in Pontejo et al. 2018 and as shown below.

As described above, the integrated iciHHV-6A genome retains the AT compositional bias distinct from that of the human host. Increased protein expression has been demonstrated for other virus genes when the biased composition is matched to that of the human host. To do this, we followed the latest compilations and predictive programs, changing the codon usage to match that of the human genome for both U83A and iciU83A as well as U83A-N and iciU83A-N cDNAs (including utilizing public databases as HIVE db). These were then further modified to adjust the poly T tract as well as any remaining of the TACC motif and the non-consensus donor/acceptor sites. These gene constructs can be used where optimum expressed protein concentration is required for example, for the uses described herein (FIG. 6a), SEQ ID NO: 24 (SEQ ID NO: 24 shows iciU83A mutated to prevent N-terminal heterogeneity, disrupt the TACC direct repeats and remove the splice donor/acceptor sites to fix the gene expression in a stable full-length agonist form with maximized human codon usage (including using HIVE db)).

### Example 2

In order to evaluate the efficacy of the novel virokine derived from iciU83A-N (referred to herein as “VTL1”, “VIT1” or “VIT”) and the novel chemokine cocktail comprising CCL5, CCL17 and CCL20 (referred to herein as “VTL3”) as an immune regulator and immunotherapeutic, these were trialled in a preclinical model of infectious disease, in order to distinguish effects on induction or inhibition of protective immunity.

Assays were conducted using a guinea pig model to evaluate the utility of the chemokine cocktail VTL3 (i.e. a mixture of mature CCL5 (SEQ ID NO: 2), mature CCL17 (SEQ ID NO: 5) and mature CCL20 (SEQ ID NO: 8)) in regulating responses to acute disease, latency, recurrences and virus infection.

### 2.1 Methods and Materials

### Viruses

Challenge virus used HSV-2 strain MS (ATCC-VR540) (Gudnadottir 1964) which was propagated primary rabbit kidney cells at low passage with subsequent titration on rabbit kidney cell monolayers, as described (Bernstein 1986).

### Vaccines

The HSV2 subunit protein vaccine was composed of truncated gD2 (306), as formulated by G. Cohen (University of Pennsylvania) from Sf9 (Spodoptera frugiperda) cells (GIBCO BRL), which were infected with a recombinant baculovirus expressing this gD2 protein as described in Willis 1998. The gD2 protein, 5 μg, was mixed with 50 μg adjuvant MPL (Sigma-Aldrich L6895), made as an aqueous formulation (Ballridge 1999) and stored at 4° C. until addition to the gD2 protein and adsorped to 500 μg aluminum hydroxide, Alhydrogel (Accurate Chemical & Scientific), as described previously in Bourne 2003. The gD2 protein was also formulated with chemokines CCL5, CCL17 and CCL20 (Peprotech) in sterile saline solution. This mixture of CCL5, CCL17 and CCL20 is referred to herein as VTL3.

For the VTL1 vaccine, 200 μg gD DNA (referred to herein as VTL2gD) was formulated with 100 μg VTL1 (SEQ ID NO: 17). The DNA constructs encoding VTL1 (mutated iciU83A-N, SEQ ID NO:17) were formulated with 0.25% bupivacaine as described (Bernstein, 1999 #120).

The DNA immunisations included formulations of HSV2 glycoprotein genes encoding gD, and human CCL5 genes were synthesized, sequence verified and expressed from independent plasmids constructed in pCMV6neo expression plasmid vector containing human CMV transcriptional 5′ enhancer, promoter and start site plus SV40 3′ polyadenylation site (Origene).

### Animals

Female Hartley guinea pigs (Charles River Laboratories), pathogen free, were used (250-350 g).

### Immunisation

In evaluating the preventative vaccine, VTL3, cohorts of n=12/group were used as follows: group 1—no vaccine; group 2—5 μg gD2 protein+MPL/Alum; group 3—5 μg gD2 protein+VTL3 (5 μg each CCL5, CCL17 and CCL20, endotoxin free, Peprotech); group 4 and 5—DNA formulations of expression plasmids containing the full length HSV2 gD DNA (200 μg VTL2gD) together with 100 μg VTL1 DNA (group 4) or CCL5 DNA (group 5). All DNA immunisations were formulated in 0.25% bupivacaine as described in Bernstein, Tepe et al., 1999). All immunisations were injected intramuscularly, i.m., followed by an identical boost after an interval of three weeks.

### Virus Challenge Preclinical Model

A day before the virus challenge, the animals were bled by toenail clipping and the subsequent separated sera stored at −20° C. Three weeks after the second immunisation, animals were challenged with virus intravaginally, i.vag., as described in Bernstein 2010. For the virus challenge, the animals were inoculated with the virus through rupture of the vaginal closure using a calcium alignate moistened tipped swab and dosing 0.1 ml of virus suspension of 10⁶ plaque forming units, PFU, of HSV2 strain MS, in order to test for evidence of induction or inhibition of immunity from the vaccine formulations as assayed by effects on acute and recurrent disease, acute and recurrent virus replication, establishment of latent infection and development of neutralising antibodies. Cervicovaginal secretions were collected by swabs on days 1, 2, 3, 8 post-inoculation (PI) then stored for assay of virus PFU on rabbit kidney cells cultivated in BME (GIBCO) and 10% FBS (Hyclone, Thermo Fisher Scientific) as described in Stanberry 1987.

Each guinea pig was examined daily and scoring conducted for primary genital skin disease. The scoring scale was from 0 to 4, with 0 indicating no disease, 1 for reddening or swelling, 2 for 1-3 small vesicles, 3 for >3 large merging lesions and 4 for several large ulcers with maceration. Animals were also evaluated from days 14-63 post-virus challenge to detect any recurrent herpetic lesions, by scoring and also totalling the number of lesions days as well as to evaluate any recurrent shedding of virus by vaginally swabbing three times per week. The swabs were stored at −80° C. until being processed for PCR analyses as a marker for virus shedding. At the completion of the study the guinea pigs were sacrificed, and dorsal root ganglia (DRG) removed aseptically, and stored at −80° C. prior to DNA extraction for evaluation by PCR for evidence of latent virus infection.

### Neutralising Antibody Assay

Serum samples were prepared in 96 well microtiter plates, 50 μl per well in serial 2-fold dilutions. Then 50 μl of HSV-2 strain MS containing 3 log 10 PFU was added to each well followed by incubation at 37° C. for 1 h with a 0.1 ml suspension of 5 log 10 BHK cells added to each well. The plates were incubated for 3 days at 37° C. in an incubator with 5% CO₂. The culture media was removed and the cells stained with crystal violet, washed and inspected for virus plaques. The effective neutralisation titre was determined as the reciprocal of the maximal serum dilution with no virus plaques, indicating 100% protection from CPE, cytopathic effect.

### Quantification of Virus DNA by PCR

DNA quantification by PCR was performed on vaginal swabs and DRG DNA. The DNA extractions were conducted as described in Bernstein et al 2010, using QIAamp DNA mini kit (QIAGEN) according to the manufacturer's protocol using tissue homogenized on ice in 500 μl of 2% FBS BME and swabs in vaginal swab media. To detect virus replication, the gB gene was amplified by PCR with 35 cycles using two sets of primers as described in Jerome 2002 and Bernstein 2010, with 50 ng purified DNA in each PCR reaction, 100 pmol each primer, Promega Master mix (PROMEGA) in a reaction volume of 25 μl.

### Statistics

Statistics were performed for all in vivo examples using Graphpad Prism with one-way ANOVA using Dunnett's test for multiple comparisons for the different vaccine treatments vs no vaccine. Where non-Gaussian distributions, non-parametric comparisons using Wilcoxon test were used. Significance was noted at P values <0.05 (*), <0.01 (**), <0.001 (***). “The immunisations were compared to the no vaccine control. Fisher's exact test was used for incidence data, with two tailed comparisons.

### 2.2 Results

### Incidence and Severity of Acute Disease

FIGS. 5 and 6 show the mean daily lesion score and total mean lesion score, respectively. FIG. 5a shows that the total lesions experienced were significantly reduced or eliminated in the VTL1 immunised animals compared to the negative control. Those without immunisation had a mean total lesion score (days 4-14 post-inoculation) for severity of 8.29(SD6.57) compared to the positive control gD protein vaccine of 0.67(SD 1.48), and the VTL1 vaccines of the immunogen formulation VTL2gD+ the VTL1 immunomodulator of 0.33(SD 0.62). These results were better than the gD subunit protein positive control immunisation and showed highly significant, almost complete, protection compared to the negative control (p<0.0001). The gD subunit protein has been clinical trialled as a vaccine, showing partial protection, so this positive control is used to compare all of the immunisation formulations. These were also markedly improved over previous experimentation using full length HSV2gD2 plasmid expressed on its own using a similar protocol, which showed total lesions score of 2.7 (+/−0.7) compared to the negative control in that experiment of 5.9(+/−0.5) (Strasser et al 2000). FIG. 6a also shows a significantly reduced total mean lesion score for animals immunised VTL1, compared to the negative control, with the VIT1 vaccine showing almost complete protection. Thus, the VTL1 DNA vaccine, containing the cDNA of human chromasomally-integrated virus-encoded chemokine-like molecule, virokine, showed efficient protection, exceeding that of the adjuvanted subunit protein vaccine previously used in clinical trials.

FIGS. 5b and 6b show that VTL3 does not significantly decrease daily mean lesions or total mean lesions in immunised animals. Thus, VTL3 blocks the immunity induced by gD2 (positive control) to acute infection. The VTL3 vaccine treatment removed the immune response elicited by gD, shown by the significantly lowered scores associated with the VTL3 formulation with gD compared to the gD-only protein vaccine.

### Effect on Virus Vaginal Replication

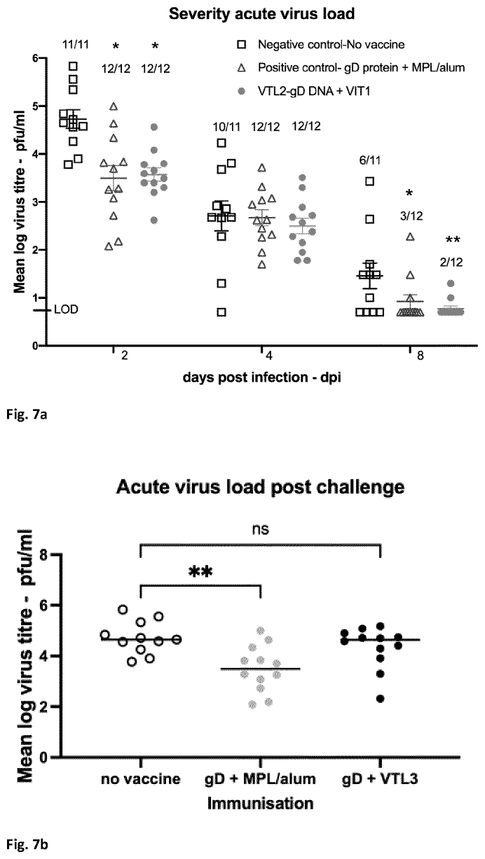

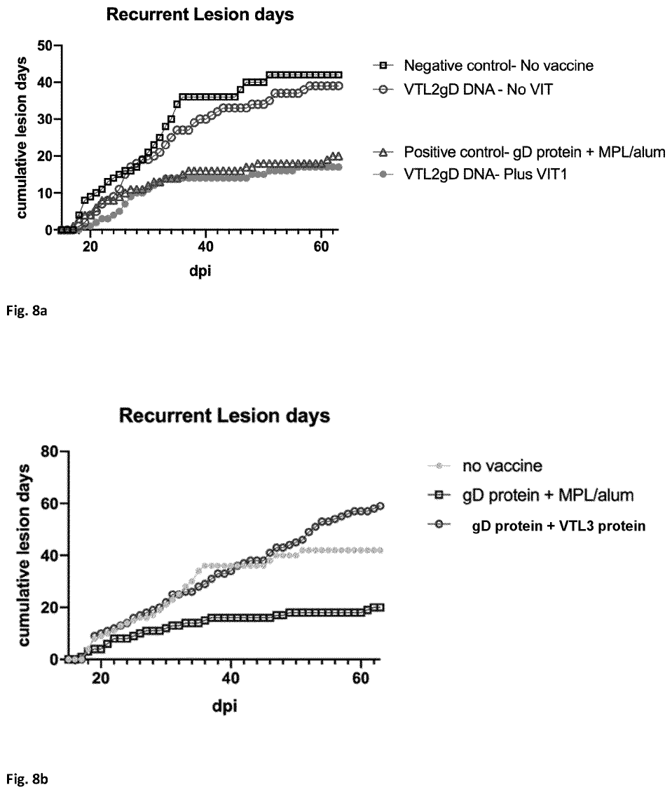

The results on the effect of immunising with the VTL1 and VTL3 formulations with gD on lesion development post-virus challenge were compared to effects on virus shedding during the primary disease. For VTL1, the analyses of the significantly lowered vaginal virus load correlates with the disease protection shown, with close to log reduction of virus shed, similar to the gD only protein formulation (positive control). The positive control (gD2/MPL-Alum protein formulation), By 8 days post virus challenge the VIT1 vaccine formulation has significantly reduced virus shedding to undetectable levels in almost all animals, p<0.01 (FIG. 7a).

In contrast, even at 2 days post-virus challenge, the VTL3 vaccine had completely blocked the reduced virus shedding effect observed with the positive control (gD only, formulated with mpl/alum) (FIG. 7b). The immunity induced against gD to prevent virus replication is blocked.

### Effects on Recurrent Disease

The in vivo preclinical HSV2 model extended follow-up to 63 days post-virus challenge after the two immunisation schedule with the vaccine formulations. In this guinea pig preclinical model of HSV2 infection, after clearance of the acute primary infection, the virus may reactivate from latency to cause recurrent disease as in people. The assays performed on the samples include DNA PCR of vaginal shedding swabs and DNA PCR of the sites for latent infection, namely, the dorsal root ganglion (DRG) and spinal cord. The efficacy endpoints were the effects on recurrent disease, asymptomatic shedding and latent viral burden. The limit on detection were marked and measured for virus quantification at 0.7 log pfu/ml and for qPCR undetectable below the limit of detection at 0.5 log microgm copies DNA/ml. Therefore, although the VTL3 was able to block protective immunity which can be induced by the gD protein, here its ability to also block immunity against recurrent infection is tested and compared to VIT (also referred to herein as “VTL1”).

### Effect in Recurrent Lesions and Lesion Days

The effects of the vaccine (VTL3) treatment on disease recurrence were analysed 15 to 63 days post-infection challenge with HSV2 virus. Cumulative daily lesions were plotted, and total mean lesion scores per individual were compared.

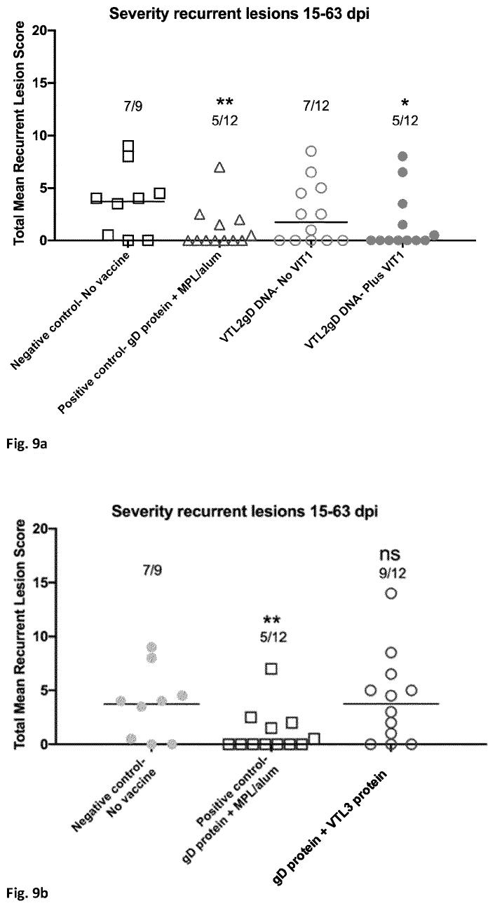

This showed that the VTL2gD DNA vaccine could only prevent recurrent disease in combination with VIT1, demonstrating the utility of VIT1 (i.e. VTL1) as a therapeutic for inducing immunity that prevented recurrent disease. Distinctly, addition of the VIT1 chemokine DNA to the VTL2gD DNA immunisation induced effective control of recurrent lesions (p<0.05). This was similar to the positive control gD protein subunit vaccine formulation (p<0.01), indicating clinical utility (FIGS. 8a and 9a). Both the VTL2gD DNA+VIT1 and gD protein subunit vaccine formulation (positive control) reduced lesion days in those with disease (FIG. 8a), while most of the animals were completely protected from any disease recurrences (7/12; 58%; FIG. 9a).

In contrast, FIGS. 8b and 9b show that immunisation with the VTL3+gD formulation removed this effect, as, despite the presence of the gD immunogen, there was no induction of immunity that reduced the lesion days or significantly reduced the number of recurrent lesions, compared to the negative control (no vaccine). In contrast, there was an increase in lesion days (FIG. 8b) compared to the negative control (no vaccine treatment).

### Effect on Virus Reactivation Shown by Asymptomatic Shedding

The effects of the vaccine treatments were tested for reductions on recurrent virus shedding after evidence for virus reactivation after day 20 post-virus challenge. To do this, the DNA load assayed in vaginal swabs by quantitative PCR was used as a surrogate for virus secretion.

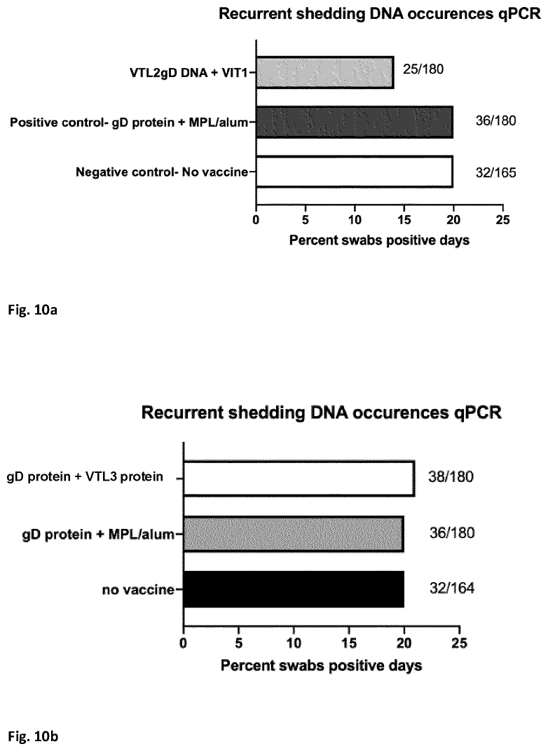

Analyses of reactivated virus in recurrent shedding events and the total mean load in vaccinated compared to unvaccinated animals were carried out. While the gD protein subunit vaccine had no effect, there was a trend for reduced virus shedding for the VTL2gD DNA+VIT formulation, and the animals who received the VIT1 vaccine had almost half the overall load (p=0.1) and a third of shedding events (20% reduced to 14 recurrences). This reduction was significant compared to the positive control (gD only protein vaccine) (FIG. 10a).

In contrast to the VIT1+gD immunisation effects, the VTL3+gD immunisation had no effect on virus shedding as this vaccine showed no significant change in recurrent shedding compared to the negative control (no vaccine; FIG. 10b).

### Effect on Latent Viral Burden

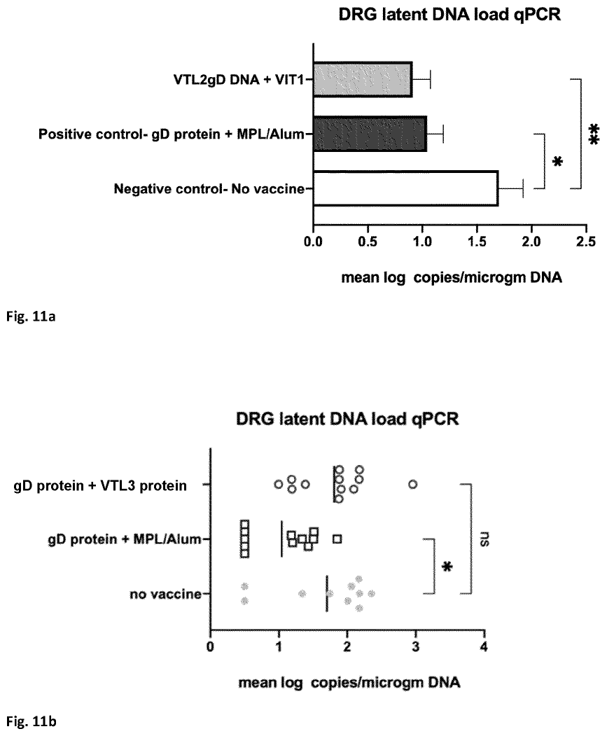

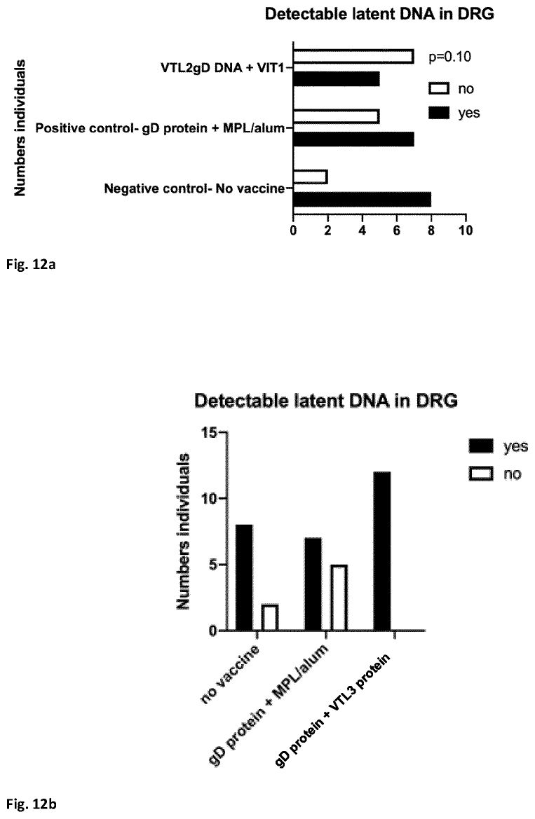

The effects of immunisation with the VIT1+gD and VTL3+gD formulations on establishment of latency at sites in the dorsal root ganglia (DRG) and the spinal cord were assayed. At the end of the study, day 63 post-virus challenge, the DNA present was quantified using qPCR at these sites of latency. Similar to the trend on virus secretion, analyses of the total mean DRG loads showed that the positive control and the VTL2gD DNA+VIT1 vaccine significantly induced immunity to reduce levels compared to the negative control, with the VTL2gD DNA+VIT1 vaccine halving amounts, p<0.01 (FIG. 11a). Over half of the animals treated with gD DNA+VIT1 were protected from detectable DNA in the DRG, compared to <20% of the animals who received no vaccine (FIG. 12a).

In contrast, immunisation with the VTL3+gD vaccine formulation did not significantly reduce the DRG latent DNA load (FIG. 11b), and VTL3 did not induce immunity to lower detectable DNA in the DRG (FIG. 12b). Even the negative control showed some animals protected from latency after a natural infection without immunisation and this effect was removed by immunisation with VTL3.

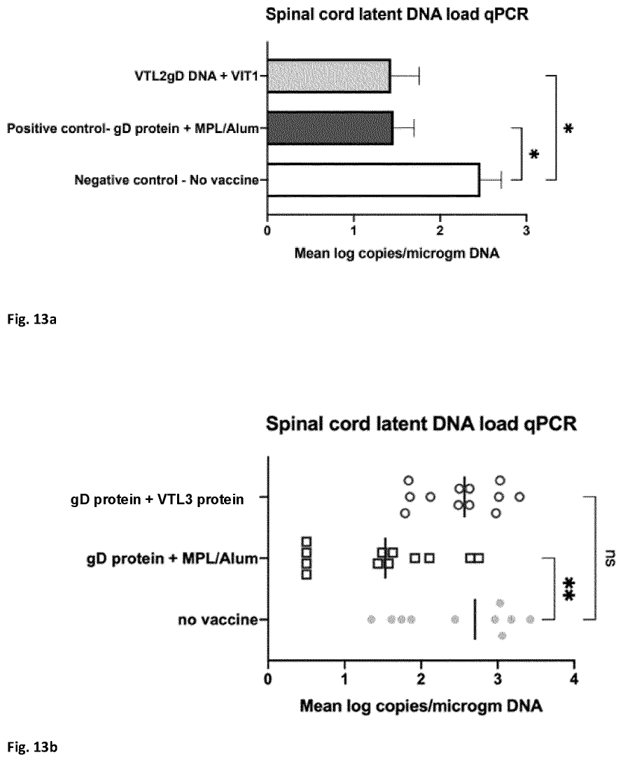

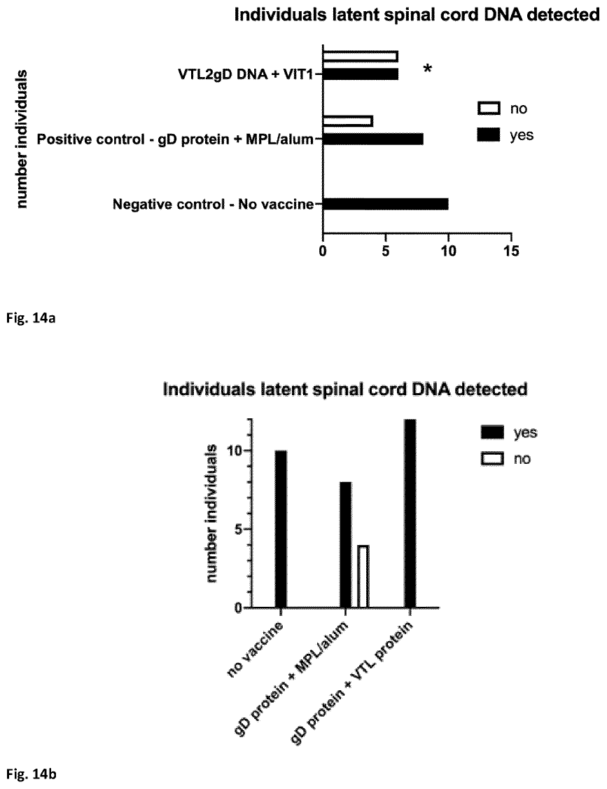

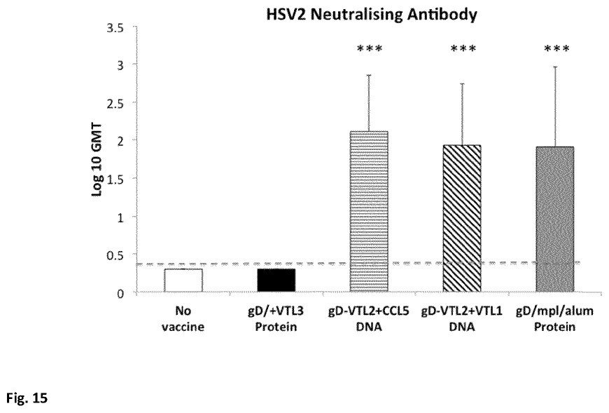

Analyses of latent DNA detected in the spinal cord showed similar effects with both the positive control (gD protein subunit) and VIT1+gD vaccine formulations significantly inducing immunity to reduce the latent DNA load in the spinal cord (p<0.05; FIG. 13a), as well as the numbers of animals harbouring latent virus DNA (FIG. 14a).

In contrast, VTL3 completely abrogated the significant reduction of latent DNA load in the spinal cord seen in the positive control (p<0.05; FIG. 13b). In addition, as shown in the latent infected DRG analyses, all animals treated with VTL3 had established latent infections which were the same as the negative control (no vaccine), despite exposure to the known efficient immunogen gD2 protein (FIG. 14b). Therefore, formulation of the gD2 immunogen with VTL3 completely inhibited all induction of immunity in this preclinical model.

### Effect on Induction of Neutralising Antibodies

The VIT1 and VTL3 gD immunisations were polar opposites in effects on inducing immunity in the guinea pig virus challenge experiment, as assayed by acute and recurrent disease protection and inhibition of acute or recurrent virus replication. Therefore, we further directly tested the ability of the VTL1+gD and VTL3+gD formulations to induce or inhibit antibody production. The gD protein immunogen (a truncated gD protein with the transmembrane region deleted) has previously been shown to effectively induce antibodies, while the addition of CpG, alum or mpl/alum induces neutralising antibodies similary (Bourne et al 2003; Awasthi et al 2017; Ghiasi et al 1994). The induction of neutralising antibodies was shown as a correlate of protection in the previous clinical trial of the gD protein formulation with mpl/alum (named ASO4), which showed partial protection (Belshe et al., 2014). This was used as the positive control in the animal model here, and has been previously shown to induce neutralising antibodies in this model (Bourne et al 2003). In previous comparisons of immunisations in the same guinea pig model, gD alone and gD with mpl/alum induced similar levels of neutralising antibodies (Bernstein et al 2010). In the immunisations with the formulations analysed herein, the gD protein+mp/alum formulation was used as a positive control and it induced effective neutralising antibodies as demonstrated previously, while the negative control (no vaccine) induced no detectable virus neutralising antibodies (FIG. 15).

The efficacy of immunisation with the VTL1+gD and VTL3+gD formulations was evaluated compared to the known immunogen gD (positive control), as described in the Materials and Methods section above.

The results (FIG. 15) showed high levels of neutralising antibody after the immunisations with either the positive control (gD2) or gD2 DNA formulated with VIT1 (i.e. VTL1). Impressively, the VTL3 formulation completely abrogated the antibody response. There was no antibody induction with the VTL3 treatment, and this gave undetectable levels similar to the negative control (no vaccine treatment). This demonstrates that VTL3 can act as a powerful immune regulator and prevent antibody stimulation even in the presence of a powerful immunogen. This appears to be a specific inhibitor as a mixture, since immunisation with gD and only one of the three chemokines in VTL3, CCL5, still results in an immunostimulant effect. Only the trivalent cytokine formulation, VTL3, showed complete abrogation of the immune response. Furthermore, the VTL1 antagonist molecule would block the chemokine receptors to which VTL3 binds, leading to maintenance of the immunostimulation.

Such a potent immune regulator formulation has clear applications in conditions of a dysregulated or aberrant immune response, such as is seen in autoimmune disease, in patients with conditions aggravated by autoimmune antibody responses, such as rheumatoid arthritis, or after chronic infectious disease such as COVID-19, rhinovirus exacerbated chronic obstructive pulmonary disease or other inflammatory conditions.

Other potential applications include the treatment or prevention of allergic responses to allergens, where the allergen could be used with the VTL3 formulation to remove immune responses to the allergen, in a vaccine to induce a type of immune tolerance. Therapeutic formulations for these conditions fulfil a growing unmet medical need, which the utility of this new formulation could address.

### 2.3 Summary

The positive control showed protection from virus challenge and efficiently induced neutralising antibodies. It is well established in preclinical animal models that gD2 functions efficiently on its own as an immunogen, as protein or DNA, and that its immunogenicity can be moderately increased with adjuvants such as MPL and alum (Bernstein et al. 1999, Bourne et al. 2003, Bernstein et al. 2010). In clinical trials, gD subunit protein vaccine with MPL and alum showed partial protection (Belshe, Leone et al. 2012).

In contrast, the evaluations here showed that the human chemokine cocktail VTL3 has a blocking effect on these activities of gD2. VTL3 was formulated to comprise the natural chemokine ligands of chemokine receptors CCR5, CCR4 and CCR6, namely CCL5 (SEQ ID NO: 2), CCL17 (SEQ ID NO: 5) and CCL20 (SEQ ID NO: 8). Therefore, VTL3 chemoattracts the T-regulatory subset by activating rather than blocking these cognate chemokine receptors. The results instead show complete blockade of all protective effects stimulated by known immunogen gD protein.

In contrast, the VTL1 formulation was highly effective against acute, primary disease and virus replication. The VTL1 vaccine had an effect on reactivated virus infections reducing recurrent virus shedding. This was not seen with the protein subunit vaccine positive control, which has previously had some efficacy in clinical trials, but required greater activity, which was provided herein by the VTL1 formulation. Also, with VTL1, there were reductions in both primary and recurrent disease, which was not seen in the absence of VTL1, as well as significant reductions in the detection of latent burden, with over half animals being completely protected. There were no adverse side effects from the immunisations; only one animal died in the study from the effects of the virus infection itself, and only in the negative control (no vaccine) group, with two further animals in this negative control (no vaccine) group having severe virus infections which prevented sample collection. In comparison, the VTL1 and VTL3 formulated vaccines were safe, showing either infection and disease protection, or removal of induced immunity, with no adverse side effects.

The results above are summarised in Table 6.

TABLE 6

| | Negative | Positive | | Immune |
|---|---|---|---|---|
| | control- | control- | | regulator - |
| | No | gD | VTL2gD | gD protein + |
| | vaccine | protein | DNA + VIT1 | VTL3 protein |
| Primary | − | + | ++ | − |
| disease | | | | |
| protection | | | | |
| Reduce | − | + | ++ | − |
| virus | | | Undetectable | |
| replication | | | d8 | |
| Reduce | − | + | + | − |
| recurrent | | | (Positive plus | |
| disease | | | VIT, Negative | |
| | | | minus VIT) | |
| Reduce | − | + | + trend | − |
| latent viral | | | (Positive plus | |
| burden | | | VIT, Negative | |
| | | | minus VIT) | |
| Induce | − | + | ++ | − |
| neutralising | | | | |
| antibodies | | | | |

The cellular recruitment offered by the virokines (VTL1) show enhanced effects on recurrences. It is well established that cellular immunity controls herpesvirus latency. In the present case, immunisation with a cellular immunodulator, VLT1, with known immunogen gD increases the immunostimulatory actions, which is consistent with the ability of the VTL1 molecule to antagonise all the chemokine receptors on T-regs, thereby preventing their recruitment to dampen down the immune response. This mode of action can block receptors present on the regulatory T-cell subsets—therefore, blocking the regulator can increase the stimulation of the immunogens. Conversely, the agonist mixture of activators of the receptors on the regulatory T-cell subsets (i.e. the chemokines in VTL3) led to inhibition of the immunogen's (gD) effects, removing induction of the immune response.

Antibody effects can prevent initial infection and can be stimulated by appropriate antigenic presentation, for example, presentation of gD2 used in the Examples above. The VTL3 immune regulatory formulation was effective in completely blocking induction of neutralising antibodies. This was also consistent with the effect of VTL3 in blocking any induced immunity from initial acute or recurrent infection, as shown herein.

VTL1 is a human adapted molecule, so its effects in a human setting, rather than guinea pigs as used herein, are likely to further improve outcomes. Comparisons of affinity for the binding domain in VTL1 with that of human chemokines showed, for example, that interactions were 10-100× increased, even using ex vivo human cells as the source of the chemokine receptors (Catusse et al 2007). In contrast to the marked inhibitory effects of VTL3, VTL1 effectively induced neutralising antibodies with known immunogen gD2. Therefore, the protective or inhibitory effects in the human system are likely to be higher for VTL1, as well as for VTL3, as a mixture of human chemokines.

As discussed above treatment with VTL3 results in blocking of immune responses and related activities to the known immunogen gD2. This warrants further investigation in a clinical setting as a preventative and therapeutic treatment for preventing immune stimulation and other runaway immune responses, such as in autoimmunity or inflammatory diseases. In particular, VTL3 attracts, and induces inhibition of the immune response to a known clinically effective immunogen (i.e. gD2) by, regulatory T-cells, as shown in the Examples above.

Examples of uses for VTL3 include treatment of pathogenic conditions with excess immune responses. For example, in rheumatoid arthritis, immune cells bearing CCR5 can be recruited to a site of disease at a joint. Therefore, blocking this recruitment of CCR5+ activated immune cells, by inducing regulatory T-cells, could be used as a beneficial treatment. Moreover, combined with known immunogens, such as allergens, unwanted immune responses, for example anaphylaxis, could be prevented by desensitising an individual by immunising the individual with the allergen together with the VTL3 formulation, in order to induce regulatory responses and inhibit pathogenic responses. There are well described allergens, such as feline hair immunogens, which could be targeted as well as common immunogens for hayfever or which provoke asthmatic attacks.

Another example of a use for VTL3 could be in the treatment of runaway immune responses to infectious disease. For example, in chronic obstructive pulmonary disease, there is an immune response to virus immunogens, such as rhinovirus, which results in obstructed airways. In this case, known immunogens from rhinovirus could be selected for immunisation with the VTL3 formulation to prevent disease. Moreover, in infections such as SARS-CoV2, severe late-stage disease is characterised by high levels of antibodies, including autoantibodies, runaway immune responses and cytokine storms, and these could be treated with an immune regulatory agent such as VTL3, to promote a regulatory response. This could be administered as an aerosol, for example, with an inhaler, to prevent local runaway immune responses in the lung, or it could be administered intramuscularly, as shown here to prevent systemic responses.

### Description of the Sequence Listing

is the amino acid sequence of the CCL5 protein precursor.
SEQ ID NO: 1
MKVSAAALAVILIATALCAPASASPYSSDTTPCCFAYIARPLPRAHIKEYFYTSGKCSNPAVVFVTRKNRQVCANP
EKKWVREYINSLEMS
is the amino acid sequence of the CCL5 mature protein.
SEQ ID NO: 2
SPYSSDTTPCCFAYIARPLPRAHIKEYFYTSGKCSNPAVVFVTRKNRQVCANPEKKWVREYINSLEMS
is the nucleotide sequence of the cDNA encoding the CCL5 mature
protein. Free text: Mature CCL5 cDNA.
SEQ ID NO: 3
tccccatatt cctcggacac cacaccctgc tgctttgcct acattgcccg cccactgccc cgtgcccaca
tcaaggagta tttctacacc agtggcaagt gctccaaccc agcagtcgtc tttgtcaccc gaaagaaccg
ccaagtgtgt gccaacccag agaagaaatg ggttcgggag tacatcaact ctttggagat gagctag
is the amino acid sequence of the CCL17 protein precursor.
SEQ ID NO: 4
MAPLKMLALVTLLLGASLQHIHAARGTNVGRECCLEYFKGAIPLRKLKTWYQTSEDCSRDAIVFVTVQGRAICSDP
NNKRVKNAVKYLQSLERS
is the amino acid sequence of the CCL17 mature protein.
SEQ ID NO: 5
ARGTNVGRECCLEYFKGAIPLRKLKTWYQTSEDCSRDAIVFVTVQGRAICSDPNNKRVKNAVKYLQSLERS
is the nucleotide sequence of the cDNA encoding the CCL17 mature
protein. Free text: Mature CCL17 cDNA.
SEQ ID NO: 6
gccccactga agatgctggc cctggtcacc ctcctcctgg gggcttctct gcagcacatc cacgcagctc
gagggaccaa tgtgggccgg gagtgctgcc tggagtactt caagggagcc attcccctta gaaagctgaa
gacgtggtac cagacatctg aggactgctc cagggatgcc atcgtttttg taactgtgca gggcagggcc
atctgttcgg accccaacaa caagagagtg aagaatgcag ttaaatacct gcaaagcctt gagaggtctt
ga
is the amino acid sequence of the CCL20 protein precursor.
SEQ ID NO: 7
MCCTKSLLLAALMSVLLLHLCGESEAASNFDCCLGYTDRILHPKFIVGFTRQLANEGCDINAIIFHTKKKLSVCAN
PKQTWVKYIVRLLSKKVKNM
is the amino acid sequence of the CCL20 mature protein.
SEQ ID NO: 8
ASNFDCCLGYTDRILHPKFIVGFTRQLANEGCDINAIIFHTKKKLSVCANPKQTWVKYIVRLLSKKVKNM
is the nucleotide sequence of the cDNA encoding the CCL20 mature
protein. Free text: Mature CCL20 cDNA.
SEQ ID NO: 9
gcaagcaact ttgactgctg tottggatac acagaccgta ttcttcatcc taaatttatt gtgggcttca
cacggcagct ggccaatgaa ggctgtgaca tcaatgctat catctttcac acaaagaaaa agttgtctgt
gtgcgcaaat ccaaaacaga cttgggtgaa atatattgtg cgtctcctca gtaaaaaagt caagaacatg
taa
is the amino acid sequence of Met-CCL5. Free text: Met-CCL5.
SEQ ID NO: 10
MSPYSSDTTPCCFAYIARPLPRAHIKEYFYTSGKCSNPAVVFVTRKNRQVCANPEKKWVREYINSLEMS
is the nucleotide sequence of the cDNA encoding Met-CCL5. Free
text: Met-CCL5 cDNA.
SEQ ID NO: 11
atgtccccat attcctcgga caccacaccc tgctgctttg cctacattgc ccgcccactg ccccgtgccc
acatcaagga gtatttctac accagtggca agtgctccaa cccagcagtc gtctttgtca cccgaaagaa
ccgccaagtg tgtgccaacc cagagaagaa atgggttcgg gagtacatca actctttgga gatgagctag
is the amino acid sequence of Met-CCL17. Free text: Met-CCL17.
SEQ ID NO: 12
MARGTNVGRECCLEYFKGAIPLRKLKTWYQTSEDCSRDAIVFVTVQGRAICSDPNNKRVKNAVKYLQSLERS
is the nucleotide sequence of the cDNA encoding Met-CCL17. Free
text: Met-CCL17 cDNA.
SEQ ID NO: 13
atggctcgag ggaccaatgt gggccgggag tgctgcctgg agtacttcaa gggagccatt ccccttagaa
agctgaagac gtggtaccag acatctgagg actgctccag ggatgccatc gtttttgtaa ctgtgcaggg
cagggccatc tgttcggacc ccaacaacaa gagagtgaag aatgcagtta aatacctgca aagccttgag
aggtcttga
is the amino acid sequence of Met-CCL20. Free text: Met-CCL20.
SEQ ID NO: 14
MASNFDCCLGYTDRILHPKFIVGFTRQLANEGCDINAIIFHTKKKLSVCANPKQTWVKYIVRLLSKKVKNM
is the nucleotide sequence of the cDNA encoding Met-CCL20. Free
text: Met-CCL20 cDNA.
SEQ ID NO: 15
atggcaagca actttgactg ctgtcttgga tacacagacc gtattcttca tcctaaattt attgtgggct
tcacacggca gctggccaat gaaggctgtg acatcaatgc tatcatcttt cacacaaaga aaaagttgtc
tgtgtgcgca aatccaaaac agacttgggt gaaatatatt gtgcgtctcc tcagtaaaaa agtcaagaac
atgtaa
is the nucleotide sequence of VTL101 iciU83A-N cDNA. Free text:
VTL101.
SEQ ID NO: 16
gtcgaaatgt ccattcggct ttttattggt tttttttata cggcatatat tggtatggct atcggattta
tatgtagttc ccccgatgcg gagctgtttt ccgaaaaatc acgtatttcg tcttctgtct tgttaggatg
tttgttgtgt tgcatggatt ggtccgctgc cgtacccgtc tggtttggag cagggctcga tgtgtga
is the nucleotide sequence of the VTL1016 variant of VTL101
iciU83A-N, mutated in the poly T tract with own Kozak sequence.
Free text: VTL1 variant VTL1016 or VIT1.
SEQ ID NO: 17
gtcgaaatgt ccattcggct ttttattggt ttcttttata cggcatatat tggtatggct atcggattta
tatgtagttc ccccgatgcg gagctgtttt ccgaaaaatc acgtatttcg tcttctgtct tgttaggatg
tttgttgtgt tgcatggatt ggtccgctgc cgtacccgtc tggtttggag cagggctcga tgtgtga
is the nucleotide sequence of the VTL1017 variant of VTL101
iciU83A-N, which has been human codon optimised and manually
adjusted with no poly T and no TACC. Free text: VTL1 variant
VTL1017
SEQ ID NO: 18
gtcgaaatgt ccatccgcct tttcattggc ttcttttaca cagcatacat cgggatggct ataggcttca
tttgctcctc tccagacgcg gagctgtttt cagagaaaag ccggatatct agtagcgtgc tgctcggatg
tctgctctgt tgcatggact ggtccgctgc cgtcccagtg tggttcggcg ctggactgga tgtgtga
is the amino acid sequence encoded by human endogenous
chromasomally-integrated human betaherpesvirus 6A VTL1,
and the VTL1016 and VTL1017 variants thereof, showing
the additional C-terminal extension of 8 amino acids.
SEQ ID NO: 19
MSIRLFIGFFYTAYIGMAIGFICSSPDAELFSEKSRISSSVLLGCLLCCMDWSAAVPVWFGAGLD
is the amino acid sequence encoded by VTL101, VTL1016 and
VTL1017 cleaved after the signal sequence to give the mature
secreted product.
SEQ ID NO: 20
FICSSPDAELFSEKSRISSSVLLGCLLCCMDWSAAVPVWFGAGLDV
is the nucleotide sequence of the VTL1018 variant of VTL101
iciU83A-N, which has been mutated to disrupt the poly T region,
and retaining own Kozak sequence. Free text: VTL1 variant
VTL1018.
SEQ ID NO: 21
gtcgaaatgt ccattcggct ttttattggt ttcttttata cggcatatat tggtatggct atcggattta
tatgtagttc ccccgatgcg gagctgtttt ccgaaaaatc acgtatttcg tottctgtct tgttaggatg
tttgttgtgt tgcatggatt ggtccgctgc cgtacctggg aaaacagagc cttttagaaa actttttgat
gcaatcatga ttaaaaagct aaaaagttgt tctgctgctt acccgtctgg tttggagcag ggctcgatgt
gtgatatggc agatgcatcg ccgacaagtc ttgaattagg attgtcgaaa ttagacaaag aatcatga
is the nucleotide sequence of the VTL1019 variant of VTL101
iciU83A-N, which has been mutated to disrupt the splicing via
the direct repeat TACC and the splice donor/acceptor sites,
and retaining Kozak consensus site. Free text: VTL1 variant
VTL1019.
SEQ ID NO: 22
gtcgaaatgt ccattcggct ttttattggt tttttttata cggcatatat tggtatggct atcggattta
tatgtagttc ccccgatgcg gagctgtttt ccgaaaaatc acgtatttcg tottctgtct tgttaggatg
tttgttgtgt tgcatggatt ggtccgctgc cgtgccaggg aaaacagagc cttttagaaa actttttgat
gcaatcatga ttaaaaagct aaaaagttgt tctgctgctt atccatctgg tttggagcag ggctcgatgt
gtgatatggc agatgcatcg ccgacaagtc ttgaattagg attgtcgaaa ttagacaaag aatcatga
is the nucleotide sequence of the VTL1020 variant of VTL101
iciU83A-N, which has been mutated to disrupt the poly T region
and the splicing, and retaining own Kozak sequence. Free text:
VTL1 variant VTL 1020
SEQ ID NO: 23
gtcgaaatgt ccattcggct ttttattggt ttcttttata cggcatatat tggtatggct atcggattta
tatgtagttc ccccgatgcg gagctgtttt ccgaaaaatc acgtatttcg tottctgtct tgttaggatg
tttgttgtgt tgcatggatt ggtccgctgc cgtgccaggg aaaacagagc cttttagaaa actttttgat
gcaatcatga ttaaaaagct aaaaagttgt tctgctgctt atccatctgg tttggagcag ggctcgatgt
gtgatatggc agatgcatcg ccgacaagtc ttgaattagg attgtcgaaa ttagacaaag aatcatga
is the nucleotide sequence of the VTL1021 variant of VTL101
iciU83A-N, which has been maximized for human codon usage and
manually adjusted to disrupt the splicing, as underlined and
retain Kozak sequence. Free text: VTL1 variant VTL1021.
SEQ ID NO: 24
gtcgaaatga gcatcagact gttcatcggc ttcttctaca ccgcctacat cggcatggcc atcggcttca
tctgcagcag ccccgacgcc gagctgttca gcgagaagag cagaatcagc agcagcgtgc tgctgggctg
cctgctgtgc tgcatggact ggagcgccgc cgtgccaggc aagaccgagc ccttcagaaa gctgttcgac
gccatcatga tcaagaagct gaagagctgc agcgccgcct atcctagcgg cctggagcag ggcagcatgt
gcgacatggc cgacgccagc cccaccagcc tggagctggg cctgagcaag ctggacaagg agagctga
is the amino acid sequence encoded by VTL1018, VTL1019, VTL1020
and VTL201. Free text: VTL1 agonist protein.
SEQ ID NO: 25
MSIRLFIGFFYTAYIGMAIGFICSSPDAELFSEKSRISSSVLLGCLLCCMDWSAAVPGKTEPFRKLFDAIMIKKLK
SCSAAYPSGLEQGSMCDMADASPTSLELGLSKLDKES
is the amino acid sequence encoded by VTL1018, VTL1019, VTL1020
and VTL201, with the signal sequence cleaved to give the mature
secreted product. Free text: VTL1 agonist mature protein.
SEQ ID NO: 26
FICSSPDAELFSEKSRISSSVLLGCLLCCMDWSAAVPGKTEPFRKLFDAIMIKKLKSCSAAYPSGLEQGSMCDMAD
ASPTSLELGLSKLDKES
is the nucleotide sequence encoding the CCL5 protein precursor,
including the signal sequence.
SEQ ID NO: 27
ATGAAGGTCTCCGCGGCAGCCCTCGCTGTCATCCTCATTGCTACTGCCCTCTGCGCTCCTGCATCTGCCTCCCCAT
ATTCCTCGGACACCACACCCTGCTGCTTTGCCTACATTGCCCGCCCACTGCCCCGTGCCCACATCAAGGAGTATTT
CTACACCAGTGGCAAGTGCTCCAACCCAGCAGTCGTCTTTGTCACCCGAAAGAACCGCCAAGTGTGTGCCAACCCA
GAGAAGAAATGGGTTCGGGAGTACATCAACTCTTTGGAGATGAGCTAG
is the nucleotide sequence encoding the CCL17 protein precursor,
including the signal sequence.
SEQ ID NO: 28
ATGGCCCCACTGAAGATGCTGGCCCTGGTCACCCTCCTCCTGGGGGCTTCTCTGCAGCACATCCACGCAGCTCGAG
GGACCAATGTGGGCCGGGAGTGCTGCCTGGAGTACTTCAAGGGAGCCATTCCCCTTAGAAAGCTGAAGACGTGGTA
CCAGACATCTGAGGACTGCTCCAGGGATGCCATCGTTTTTGTAACTGTGCAGGGCAGGGCCATCTGTTCGGACCCC
AACAACAAGAGAGTGAAGAATGCAGTTAAATACCTGCAAAGCCTTGAGAGGTCTTGA
is the nucleotide sequence encoding the CCL20 protein precursor,
including the signal sequence.
SEQ ID NO: 29
ATGTGCTGTACCAAGAGTTTGCTCCTGGCTGCTTTGATGTCAGTGCTGCTACTCCACCTCTGCGGCGAATCAGAAG
CAGCAAGCAACTTTGACTGCTGTCTTGGATACACAGACCGTATTCTTCATCCTAAATTTATTGTGGGCTTCACACG
GCAGCTGGCCAATGAAGGCTGTGACATCAATGCTATCATCTTTCACACAAAGAAAAAGTTGTCTGTGTGCGCAAAT
CCAAAACAGACTTGGGTGAAATATATTGTGCGTCTCCTCAGTAAAAAAGTCAAGAACATGTAA

### REFERENCES

Awasthi S, Hook L M, Shaw C E, Pahar B, Stagray J A, Liu D, Veazey R S, Friedman H M. “An HSV-2 Trivalent Vaccine Is Immunogenic in Rhesus Macaques and Highly Efficacious in Guinea Pigs”. PLoS Pathog. (2017) January 19; 13(1):e1006141. doi: 10.1371/journal.ppat.1006141. eCollection 2017 Jan.PMID: 28103319.
Baldridge J R, Crane R T. Monophosphoryl lipid A (MPL) formulations for the next generation of vaccines. Methods 1999; 19:103-7.
Bar-On, Y. M., Y. Goldberg, M. Mandel, O. Bodenheimer, L. Freedman, N. Kalkstein, B. Mizrahi, S. Alroy-Preis, N. Ash, R. Milo and A. Huppert (2021). “Protection of BNT162b2 Vaccine Booster against Covid-19 in Israel.” N Engl J Med. September 15:NEJMoa2114255. doi: 10.1056/NEJMoa2114255. PMID: 34525275.
Barsheshet, Y., G. Wildbaum, E. Levy, A. Vitenshtein, C. Akinseye, J. Griggs, S. A. Lira and N. Karin (2017). “CCR8(+)FOXp3(+) Treg cells as master drivers of immune regulation.” Proc Natl Acad Sci USA 114(23): 6086-6091.
Bayry, J., E. Z. Tchilian, M. N. Davies, E. K. Forbes, S. J. Draper, S. V. Kaveri, A. V. Hill, M. D. Kazatchkine, P. C. Beverley, D. R. Flower and D. F. Tough (2008). “In silico identified CCR4 antagonists target regulatory T cells and exert adjuvant activity in vaccination.” Proc Natl Acad Sci USA 105(29): 10221-10226.
Belshe, R. B., P. A. Leone, D. I. Bernstein, A. Wald, M. J. Levin, J. T. Stapleton, I. Gorfinkel, R. L. Morrow, M. G. Ewell, A. Stokes-Riner, G. Dubin, T. C. Heineman, J. M. Schulte, C. D. Deal and W. Herpevac Trial for (2012). “Efficacy results of a trial of a herpes simplex vaccine.” N Engl J Med 366(1): 34-43.
Belshe B., Heineman T. C., Bernstein D. I., Bellamy A. R., Ewell M., van der Most R., et al. “Correlate of immune protection against HSV-1 genital disease in vaccinated women”, J Infect Dis (2014), Vol. 209, Issue 6, Pages 828-36.
Bendtsen, J. D., Nielsen, H., von Heijne, G. and Brunak, S, Improved prediction of signal peptides: SIgnaIP 3.0, Journal of Molecular Biology, 16 Jul. 2004, 340(4), 783-795.
Bernstein D I, Stanberry L R, Harrison C J, Kappes J C, Myers M G. Antibody response, recurrence patterns and subsequent herpes simplex virus type 2 (HSV-2) re-infection following initial HSV-2 infection of guinea-pigs: effects of acyclovir. J General Virol (1986); 67(Pt 8):1601-12.
Bernardini G., Hedrick J., Sozzani S., Luini W., Spinetti G., Weiss M., “Identification of the C C chemokines TARC and macrophage inflammatory protein-1 beta as novel functional ligands for the CCR8 receptor”, Eur J Immunol 1998 Vol. 28 Issue 2 Pages 582-8,
Bernstein D I, Tepe E R, Mester J C, Arnold R L, Stanberry L R, Higgins T. “Effects of DNA immunization formulated with bupivacaine in murine and guinea pig models of genital herpes simplex virus infection. Vaccine. 1999 Apr. 9; 17(15-16):1964-9.
Bernstein D I, Farley N, Bravo F J, Earwood J, McNeal M, Fairman J, and Cardin R. “The Adjuvant CLDC Increases Protection of a Herpes Simplex Type 2 Glycoprotein D Vaccine in Guinea Pigs”. Vaccine (2010); 28: 3748-3753.
Bobanga, I. D., Petrosiute, A., and Huang, A. Y. (2013). Chemokines as Cancer Vaccine Adjuvants. Vaccines (Basel) 1, 444-462.
Bourne N, Bravo F J, Francotte M, Bernstein D I, Myers M G, Slaoui M, Stanberry L R. “Herpes simplex virus (HSV) type 2 glycoprotein D subunit vaccines and protection against genital HSV-1 or HSV-2 disease in guinea pigs”. J Infect Dis. 2003 Feb. 15; 187(4):542-9. doi: 10.1086/374002. Epub 2003 Feb. 7.
Catusse, J., C. M. Parry, D. R. Dewin and U. A. Gompels (2007). “Inhibition of HIV-1 infection by viral chemokine U83A via high-affinity CCR5 interactions that block human chemokine-induced leukocyte chemotaxis and receptor internalization.” Blood 109(9): 3633-3639.
Catusse, J., Clark, D. J., and Gompels, U. A. (2009). CCR5 signalling, but not DARC or D6 regulatory, chemokine receptors are targeted by herpesvirus U83A chemokine which delays receptor internalisation via diversion to a caveolin-linked pathway. J Inflamm (Lond) 6, 22.
Catusse, J., Parry, C. M., Dewin, D. R., and Gompels, U. A. (2007). “Inhibition of HIV-1 infection by viral chemokine U83A via high-affinity CCR5 interactions that block human chemokine-induced leukocyte chemotaxis and receptor internalization”. Blood 109, 3633-3639.
Clark, D. J., Catusse, J., Stacey, A., Borrow, P., and Gompels, U. A. (2013). “Activation of CCR2+ human proinflammatory monocytes by human herpesvirus-6B chemokine N-terminal peptide”. J Gen Virol 94, 1624-1635.
Dewin, D. R., Catusse, J., and Gompels, U. A. (2006). “Identification and characterization of U83A viral chemokine, a broad and potent beta-chemokine agonist for human CCRs with unique selectivity and inhibition by spliced isoform”. J Immunol 176, 544-556.
Dong, X., C. Wang, X. Liu, W. Gao, X. Bai and Z. Li (2020). “Lessons Learned Comparing Immune System Alterations of Bacterial Sepsis and SARS-CoV-2 Sepsis.” Front Immunol 11: 598404.
Eyre, D. W., D. Taylor, M. Purver, D. Chapman, R. Fowler, K. B. Pouwels, A. S. Walker and T. E. A. Peto (2021). “The impact of SARS-CoV-2 vaccination on Alpha & Delta variant transmission.” MedRxiv.
J. M. Fox, P. Najarro, G. L. Smith, S. Struyf, P. Proost and J. E. Pease. “Structure/function relationships of CCR8 agonists and antagonists. Amino-terminal extension of CCL1 by a single amino acid generates a partial agonist”, J Biol Chem 2006 Vol. 281 Issue 48 Pages 36652-61. Accession Number: 17023422 DOI: 10.1074/jbc.M605584200. https://www.ncbi.nlm.nih.gov/pubmed/17023422.
French, C., Menegazzi, P., Nicholson, L., Macaulay, H., DiLuca, D., and Gompels, U. A. (1999). “Novel, nonconsensus cellular splicing regulates expression of a gene encoding a chemokine-like protein that shows high variation and is specific for human herpesvirus 6”. Virology 262, 139-151.
Ghiasi H, Kaiwar R, Nesburn A B, Slanina S, Wechsler S L. “Expression of seven herpes simplex virus type 1 glycoproteins (gB, gC, gD, gE, gG, gH, and gI): comparative protection against lethal challenge in mice”. J Virol. 1994 April; 68(4):2118-26. doi: 10.1128/JVI.68.4.2118-2126.1994.
Greninger, A. L., Knudsen, G. M., Roychoudhury, P., Hanson, D. J., Sedlak, R. H., Xie, H., Guan, J., Nguyen, T., Peddu, V., Boeckh, M., et al. (2018). Comparative genomic, transcriptomic, and proteomic reannotation of human herpesvirus 6. BMC Genomics 19, 204.
Gudnadottir M, Helgadottir H, Bjarnason O, Jonsdottir K. Virus isolated from the brain of a patient with multiple sclerosis. Exp Neurol 1964; 9:85-95.
Hughes, C. E. and R. J. B. Nibbs (2018). “A guide to chemokines and their receptors.” FEBS J 285(16): 2944-2971.
Iellem, A., M. Mariani, R. Lang, H. Recalde, P. Panina-Bordignon, F. Sinigaglia and D. D'Ambrosio (2001). “Unique chemotactic response profile and specific expression of chemokine receptors CCR4 and CCR8 by CD4(+)CD25(+) regulatory T cells.” J Exp Med 194(6): 847-853.
Ikebuchi, R., M. Fujimoto, Y. Nakanishi, H. Okuyama, T. Moriya, Y. Kusumoto and M. Tomura (2019). “Functional Phenotypic Diversity of Regulatory T Cells Remaining in Inflamed Skin.” Front Immunol 10: 1098.
Jerome K R, Huang M L, Wald A, Selke S, Corey L. Quantitative stability of DNA after extended storage of clinical specimens as determined by real-time PCR. J Clin Microbiol 2002 July; 40(7):2609-11.
Kallikourdis, M., K. G. Andersen, K. A. Welch and A. G. Betz (2007). “Alloantigen-enhanced accumulation of CCR5+ ‘effector’ regulatory T cells in the gravid uterus.” Proc Natl Acad Sci USA 104(2): 594-599.
Kamei, N., Tamiwa, H., Miyata, M., Haruna, Y., Matsumura, K., Ogino, H., Hirano, S., Higashiyama, K., and Takeda-Morishita, M. (2018). Hydrophobic Amino Acid Tryptophan Shows Promise as a Potential Absorption Enhancer for Oral Delivery of Biopharmaceuticals. Pharmaceutics 10.
Karin, N. (2018). “Chemokines and cancer: new immune checkpoints for cancer therapy.” Curr Opin Immunol 51: 140-145.
Katou, F., H. Ohtani, T. Nakayama, K. Ono, K. Matsushima, A. Saaristo, H. Nagura, O. Yoshie and K. Motegi (2001). “Macrophage-derived chemokine (MDC/CCL22) and CCR4 are involved in the formation of T lymphocyte-dendritic cell clusters in human inflamed skin and secondary lymphoid tissue.” Am J Pathol 158(4): 1263-1270.
Korbecki, J., S. Grochans, I. Gutowska, K. Barczak and I. Baranowska-Bosiacka (2020). “CC Chemokines in a Tumor: A Review of Pro-Cancer and Anti-Cancer Properties of Receptors CCR5, CCR6, CCR7, CCR8, CCR9, and CCR10 Ligands.” Int J Mol Sci 21(20).
Korbecki, J., K. Kojder, D. Siminska, R. Bohatyrewicz, I. Gutowska, D. Chlubek and I. Baranowska-Bosiacka (2020). “CC Chemokines in a Tumor: A Review of Pro-Cancer and Anti-Cancer Properties of the Ligands of Receptors CCR1, CCR2, CCR3, and CCR4.” Int J Mol Sci 21(21).
Lin, C. F., Mount, S. M., Jarmolowski, A., and Makalowski, W. (2010). Evolutionary dynamics of U12-type spliceosomal introns. BMC Evol Biol 10, 47.
Lu, L., J. Barbi and F. Pan (2017). “The regulation of immune tolerance by FOXP3.” Nat Rev Immunol 17(11): 703-717.
Luo, C. T. and M. O. Li (2018). “Foxo transcription factors in T cell biology and tumor immunity.” Semin Cancer Biol 50: 13-20.
Mikhak, Z., Fukui, M., Farsidjani, A., Medoff, B. D., Tager, A. M., and Luster, A. D. “Contribution of CCR4 and CCR8 to antigen-specific T(H)2 cell trafficking in allergic pulmonary inflammation”. J Allergy Clin Immunol 2009, 123, 67-73. e3Accession Number: 19062085 PMCID: PMC2782398 DOI: 10.1016/j.jaci.2008.09.049.
Miyara, M. and S. Sakaguchi (2007). “Natural regulatory T cells: mechanisms of suppression.” Trends Mol Med 13(3): 108-116.
Mohan, T., W. Zhu, Y. Wang and B. Z. Wang (2018). “Applications of chemokines as adjuvants for vaccine immunotherapy.” Immunobiology 223(6-7): 477-485.
Mohr, A., M. Atif, R. Balderas, G. Gorochov and M. Miyara (2019). “The role of FOXP3(+) regulatory T cells in human autoimmune and inflammatory diseases.” Clin Exp Immunol 197(1): 24-35.
Nielsen H., sirigos, K. D., Brunak, S. and von Heijne, G, “A brief history of protein sorting prediction”. The Protein Journal, 22 May 2019, 38:200-216.
Ohue, Y. and H. Nishikawa (2019). “Regulatory T (Treg) cells in cancer: Can Treg cells be a new therapeutic target?” Cancer Sci 110(7): 2080-2089.
Peligero-Cruz, C., T. Givony, A. Sebe-Pedros, J. Dobes, N. Kadouri, S. Nevo, F. Roncato, R. Alon, Y. Goldfarb and J. Abramson (2020). “IL18 signaling promotes homing of mature Tregs into the thymus.” Elife 9. DOI: 10.7554/eLife.58213.
Pontejo, S. M., Murphy, P. M., and Pease, J. E. (2018). Chemokine Subversion by Human Herpesviruses. J Innate Immun 10, 465-478.
Proudfoot A E, Power C A, Hoogewerf A J, Montjovent M O, Borlat F, Offord R E and Wells T N, J Biol Chem, “Extension of recombinant human RANTES by the retention of the initiating methionine produces a potent antagonist”, J Biol Chem., 1996 Feb. 2; 271(5):2599-603. doi: 10.1074/jbc.271.5.2599.
Proudfoot A E, Buser R, Borlat F, Alouani S, Soler D, Offord R E, Schröder J and Wells T N, “Amino-terminally modified RANTES analogues demonstrate differential effects on RANTES receptors”, Protein Cemistry and Structure, 5 Nov. 1999, vol. 274(45), p. 32478-32485.
Proudfoot A E & Uguccioni M, “Modulation of chemokine responses: synergy and cooperativity”, Frontiers in Immunology, 19 May 2016, vol. 7, article 183.
Schlecker, E., A. Stojanovic, C. Eisen, C. Quack, C. S. Falk, V. Umansky and A. Cerwenka (2012). “Tumor-infiltrating monocytic myeloid-derived suppressor cells mediate CCR5-dependent recruitment of regulatory T cells favoring tumor growth.” J Immunol 189(12): 5602-5611.
Singh, S., Datta, A., Schmidtchen, A., Bhunia, A., and Malmsten, M. (2017). “Tryptophan end-tagging for promoted lipopolysaccharide interactions and anti-inflammatory effects”. Sci Rep 7, 212.
Snelgrove, S. L., L. D. Abeynaike, S. Thevalingam, J. A. Deane and M. J. Hickey (2019). “Regulatory T Cell Transmigration and Intravascular Migration Undergo Mechanistically Distinct Regulation at Different Phases of the Inflammatory Response.” J Immunol 203(11): 2850-2861.
Stanberry, L. R., Bernstein, D. I., Burke, R. L., Pachl, C., Myers, M. G. “Vaccination with recombinant herpes simplex virus glycoproteins: protection against initial and recurrent genital herpes”. J Infect Dis (1987) May; 155(5):914-20. [PubMed: 3031173].
Strasser, J. E., Arnold, R. L., Pachuk, C., Higgins, T. J., and Bernstein, D. I. (2000). “Herpes simplex virus DNA vaccine efficacy: effect of glycoprotein D plasmid constructs”. J Infect Dis 182, 1304-1310.
Tang, H. L. and J. G. Cyster (1999). “Chemokine Up-regulation and activated T cell attraction by maturing dendritic cells.” Science 284(5415): 819-822.
Tweedy, J., Spyrou, M. A., Hubacek, P., Kuhl, U., Lassner, D., and Gompels, U. A. (2015). “Analyses of germline, chromosomally integrated human herpesvirus 6A and B genomes indicate emergent infection and new inflammatory mediators”. J Gen Virol 96, 370-389.
Tweedy, J., Spyrou, M. A., Pearson, M., Lassner, D., Kuhl, U., and Gompels, U. A. (2016). “Complete Genome Sequence of Germline Chromosomally Integrated Human Herpesvirus 6A and Analyses Integration Sites Define a New Human Endogenous Virus with Potential to Reactivate as an Emerging Infection”. Viruses 8.
Tweedy, J. G., Escriva, E., Topf, M., and Gompels, U. A. (2017). Analyses of Tissue Culture Adaptation of Human Herpesvirus-6A by Whole Genome Deep Sequencing Redefines the Reference Sequence and Identifies Virus Entry Complex Changes. Viruses 10.
Vilgelm, A. E., and Richmond, A. (2019). “Chemokines Modulate Immune Surveillance in Tumorigenesis, Metastasis, and Response to Immunotherapy”. Front Immunol 10, 333.
Villarreal, D. O., A. L'Huillier, S. Armington, C. Mottershead, E. V. Filippova, B. D. Coder, R. G. Petit and M. F. Princiotta (2018). “Targeting CCR8 Induces Protective Antitumor Immunity and Enhances Vaccine-Induced Responses in Colon Cancer.” Cancer Res 78(18):5340-5348.
Willis S H, Rux A H, Peng C, Whitbeck J C, Nicola A V, Lou H, et al. Examination of the kinetics of herpes simplex virus glycoprotein D binding to the herpesvirus entry mediator, using surface plasmon resonance. J Virol (1998); 72:5937-47.
Wu, M., H. Fang and S. T. Hwang (2001). “Cutting edge: CCR4 mediates antigen-primed T cell binding to activated dendritic cells.” J Immunol 167(9): 4791-4795.
Yau, W. M., Wimley, W. C., Gawrisch, K., and White, S. H. (1998). “The preference of tryptophan for membrane interfaces”. Biochemistry 37; 14713-14718.

1. A chemokine mixture comprising a first chemokine, or an agonist or antagonist variant thereof, which is suitable for binding to CCR4 and a second chemokine, or an agonist or antagonist variant thereof, which is suitable for binding to CCR5 and/or CCR6, wherein the chemokine mixture is suitable for modulating an immune response.

2. The chemokine mixture according to claim 1, wherein the first chemokine or agonist or antagonist variant thereof comprises CCL17 or an agonist or antagonist variant thereof, wherein preferably the agonist or antagonist variant thereof comprises an amino acid sequence which has at least 70% sequence identity to SEQ ID NO: 5, or SEQ ID NO: 12.

3. The chemokine mixture according to claim 1, wherein the second chemokine or agonist or antagonist variant thereof comprises CCL5 or CCL20 or an agonist or antagonist variant thereof, wherein preferably the agonist or antagonist variant thereof comprises an amino acid sequence which has at least 70% sequence identity to one of SEQ ID NOs: 2, 8, 10, 14 or 26.

4. (canceled)

5. The chemokine mixture according to claim 1, wherein the chemokine mixture comprises a third chemokine or an agonist or antagonist variant thereof, wherein the third chemokine or agonist or antagonist variant thereof is suitable for binding to a different one of CCR5 and CCR6 from the second chemokine or agonist or antagonist variant thereof.

6. The chemokine mixture according to claim 5, wherein the third chemokine or agonist or antagonist variant thereof comprises CCL20 or an agonist or antagonist variant thereof, or comprises an amino acid sequence which has at least 70% sequence identity to SEQ ID NO: 8 or SEQ ID NO: 14.

7. (canceled)

8. The chemokine mixture according to claim 5, wherein:
a) the first chemokine comprises CCL17, the second chemokine comprises CCL5 or SEQ ID NO: 26, and the chemokine mixture comprises a third chemokine which comprises CCL20; or
b) the first chemokine comprises Met-CCL17, the second chemokine comprises Met-CCL5 or SEQ ID NO: 20, and the chemokine mixture comprises a third chemokine which comprises Met-CCL20, or
c) the first chemokine comprises CCL17, the second chemokine comprises Met-CCL5 or SEQ ID NO: 20, and the chemokine mixture comprises a third chemokine which comprises CCL20.

9. (canceled)

10. A polynucleotide mixture comprising a first polynucleotide and a second polynucleotide, wherein the first polynucleotide comprises a nucleic acid sequence which encodes a first chemokine, or an agonist or antagonist variant thereof, which is suitable for binding to CCR4, and is optionally also suitable for binding to CCR8, and the second polynucleotide comprises a nucleic acid sequence which encodes a second chemokine, or an agonist or antagonist variant thereof, which is suitable for binding to CCR5 and/or CCR6, wherein the polynucleotide mixture is suitable for modulating an immune response.

11. The polynucleotide mixture according to claim 10, wherein the first polynucleotide comprises a nucleic acid sequence which encodes CCL17 or an agonist or antagonist variant thereof, and wherein preferably the first polynucleotide further comprises a nucleic acid sequence which encodes a signal sequence.

12. The polynucleotide mixture according to claim 10, wherein the second polynucleotide comprises a nucleic acid sequence which encodes CCL5 or an agonist or antagonist variant thereof or CCL20, or an agonist or antagonist variant thereof, or one of SEQ ID NOs: 20 and 26, and wherein preferably the second polynucleotide further comprises a nucleic acid sequence which encodes a signal sequence.

13. The polynucleotide mixture according to claim 10, wherein the first polynucleotide comprises a nucleic acid sequence which has at least 70% sequence identity to one of SEQ ID NOs: 6 and 13, and wherein the second polynucleotide comprises a nucleic acid sequence which has at least 70% sequence identity to one of SEQ ID NOs: 3, 9, 11, 15, 16-18 and 21-24, and wherein preferably the first polynucleotide and the second polynucleotide further comprises a nucleic acid sequence which encodes a signal sequence.

14. The polynucleotide mixture according to claim 10 wherein the second chemokine or functional variant thereof is suitable for binding to CCL5 and the polynucleotide mixture comprises a third polynucleotide, wherein the third polynucleotide comprises a nucleic acid sequence which encodes a third chemokine, or an agonist or antagonist variant thereof, wherein the third chemokine or agonist or antagonist variant thereof is suitable for binding to CCR6, and wherein preferably the third polynucleotide further comprises a nucleic acid sequence which encodes a signal sequence.

15. The polynucleotide mixture according to claim 14, wherein the third chemokine or agonist or antagonist variant thereof comprises CCL20 or an agonist or antagonist variant thereof or wherein the third polynucleotide comprises a nucleic acid sequence which has at least 70% sequence identity to SEQ ID NO: 13 or SEQ ID NO: 15, and wherein preferably the third polynucleotide further comprises a nucleic acid sequence which encodes a signal sequence.

16. (canceled)

17. The polynucleotide mixture according to claim 10, wherein:
a) the first polynucleotide comprises a nucleic acid sequence which encodes CCL17, the second polynucleotide comprises a nucleic acid sequence which encodes CCL5 or which has an amino acid sequence which has at least 70% sequence identity to one of SEQ ID NO: 21-24, and the polynucleotide mixture comprises a third polynucleotide, wherein the third polynucleotide comprises a nucleic acid sequence which encodes CCL20; or
b) the first polynucleotide comprises a nucleic acid sequence which encodes Met-CCL17, the second polynucleotide comprises a nucleic acid sequence which encodes Met-CCL5 or which has at least 70% sequence identity to one of SEQ ID NOs: 16-18, and the polynucleotide mixture comprises a third polynucleotide, wherein the third polynucleotide comprises a nucleic acid sequence which encodes Met-CCL20, or
c) the first polynucleotide comprises an amino acid sequence which encodes CCL17, the second polynucleotide which encodes Met-CCL5 or which comprises an amino acid sequence which has at least 70% sequence identity to one of SEQ ID NOs: 16-18, and the polynucleotide mixture comprises a third polynucleotide, wherein the third polynucleotide comprises a nucleic acid sequence which encodes CCL20;
and wherein preferably the first polynucleotide and the second polynucleotide and the third polynucleotide further comprises a nucleic acid sequence which encodes a signal sequence.

18. The polynucleotide mixture according to claim 10, wherein:
a) the first polynucleotide comprises a nucleic acid sequence which has at least 70% sequence identity to SEQ ID NO. 6, the second polynucleotide comprises a nucleic acid sequence which has at least 70% sequence identity to one of SEQ ID NOs: 3 and 21-24, and the polynucleotide mixture comprises a third polynucleotide, wherein the third polynucleotide comprises a nucleic acid sequence which at least 70% sequence identity to SEQ ID NO: 9; or
b) the first polynucleotide comprises a nucleic acid sequence which has at least 70% sequence identity to SEQ ID NO: 13, the second polynucleotide comprises a nucleic acid sequence which has at least 70% sequence identity to one of SEQ ID NOs: 11 and 16-18, and the polynucleotide mixture comprises a third polynucleotide, wherein the third polynucleotide comprises a nucleic acid sequence which has at least 70% sequence identity to SEQ ID NO: 15, or
c) the first polynucleotide comprises an amino acid sequence which has at least 70% sequence identity to SEQ ID NO: 6, the second polynucleotide comprises an amino acid sequence which has at least 70% sequence identity to one of SEQ ID NOs: 11 and 16-18, and the polynucleotide mixture comprises a third polynucleotide, wherein the third polynucleotide comprises a nucleic acid sequence which has at least 70% sequence identity to SEQ ID NO: 9;
and wherein preferably the first polynucleotide and the second polynucleotide and the third polynucleotide further comprises a nucleic acid sequence which encodes a signal sequence.

19. (canceled)

20. A composition comprising a first polynucleotide comprising a nucleic acid sequence which encodes a first chemokine or an agonist or antagonist variant thereof which is suitable for binding to one of CCR5, CCR4 and/or CCR6, and comprising a second chemokine or an agonist or antagonist variant thereof which is suitable for binding to a different one of CCR5, CCR4 and/or CCR6 from the first chemokine or agonist or antagonist thereof, wherein the composition is suitable for modulating an immune response.

21-22. (canceled)

23. The composition of claim 20, wherein the first polynucleotide encodes CCL5, CCL17, CCL20, or an agonist or antagonist variant thereof, or encodes an amino acid sequence having at least 70% sequence identity to one of SEQ ID Nos: 20 and 26, or comprises a nucleic acid sequence which has at least 70% sequence identity to one of SEQ ID NOs: 3, 6, 9, 11, 13, 15-18 and 21-24, and wherein preferably the first polynucleotide further comprises a nucleic acid sequence which encodes a signal sequence.

24. The composition of claim 20, wherein the second chemokine or agonist or antagonist variant thereof comprises CCL5, CCL17, CCL20, or an agonist or antagonist variant thereof, or comprises an amino acid sequence which has at least 70% sequence identity to one of SEQ ID Nos: 20 and 26, or wherein the composition comprises a second polynucleotide, wherein the second polynucleotide comprises a nucleic acid sequence which has at least 70% sequence identity to a different one of SEQ ID NOs: 3, 6, 9, 11, 13, 15-18 and 21-24 from the first polynucleotide, and wherein preferably the second polynucleotide further comprises a nucleic acid sequence which encodes a signal sequence.

25. The composition of claim 20, wherein the composition comprises a second polynucleotide which comprises a nucleic acid sequence which encodes a third chemokine or agonist or antagonist variant thereof, or the composition comprises a third chemokine or agonist or antagonist variant thereof, wherein the third chemokine or agonist or antagonist variant thereof is suitable for binding to a different one of CCR5, CCR4 and/or CCR6 from each of the first and second chemokines or agonist or antagonist variants thereof, and optionally wherein the third chemokine or agonist or antagonist variant thereof comprises CCL5, CCL17, CCL20, or wherein the third chemokine or agonist or antagonist variant thereof comprises an amino acid sequence which has at least 70% sequence identity to one of SEQ ID NOs: 20 and 26, and wherein preferably the second polynucleotide, and optionally the third polynucleotide, further comprises a nucleic acid sequence which encodes a signal sequence.

26-28. (canceled)

29. The composition of claim 20, wherein the first chemokine or agonist or antagonist variant thereof comprises CCL5 or an agonist or antagonist variant thereof, or comprises an amino acid sequence having at least 70% sequence identity to one of SEQ ID NOs: 20 and 26, and the second chemokine or agonist or antagonist variant thereof comprises CCL17 or an agonist or antagonist variant thereof, and the composition comprises a third chemokine or agonist or antagonist variant thereof which comprises CCL20 or an agonist or antagonist variant thereof.

30-31. (canceled)

32. The composition of claim 29, wherein the first chemokine, or agonist or antagonist variant thereof, comprises an amino acid sequence having at least 70% sequence identity to SEQ ID NO: 20, the second chemokine or agonist or antagonist variant thereof is Met-CCL17 and the third chemokine or agonist or antagonist variant thereof is Met-CCL20.

33-72. (canceled)


SEQUENCE LISTING (29 sequences)

SEQ ID NO: 1 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 2 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 3 - DNA, 207 bp, synthetic construct
  misc_feature 1..207: note = Mature CCL5 cDNA
  source 1..207: mol_type = other DNA
tccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 4 - protein, 94 aa, Homo sapiens
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 5 - protein, 71 aa, Homo sapiens
ARGTNVGRECCLEYFKGAIPLRKLKTWYQTSEDCSRDAIVFVTVQGRAICSDPNNKRVKNAVKYLQSLERS

SEQ ID NO: 6 - DNA, 216 bp, synthetic construct
  misc_feature 1..216: note = Mature CCL17 cDNA
  source 1..216: mol_type = other DNA
gctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttga

SEQ ID NO: 7 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 8 - protein, 70 aa, Homo sapiens
ASNFDCCLGYTDRILHPKFIVGFTRQLANEGCDINAIIFHTKKKLSVCANPKQTWVKYIVRLLSKKVKNM

SEQ ID NO: 9 - DNA, 213 bp, synthetic construct
  misc_feature 1..213: note = Mature CCL20 cDNA
  source 1..213: mol_type = other DNA
gcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaa

SEQ ID NO: 10 - protein, 69 aa, synthetic construct
  REGION 1..69: note = Met-CCL5
MSPYSSDTTPCCFAYIARPLPRAHIKEYFYTSGKCSNPAVVFVTRKNRQVCANPEKKWVREYINSLEMS

SEQ ID NO: 11 - DNA, 210 bp, synthetic construct
  misc_feature 1..210: note = Met-CCL5 cDNA
  source 1..210: mol_type = other DNA
atgtccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 12 - protein, 72 aa, synthetic construct
  REGION 1..72: note = Met-CCL17
MARGTNVGRECCLEYFKGAIPLRKLKTWYQTSEDCSRDAIVFVTVQGRAICSDPNNKRVKNAVKYLQSLERS

SEQ ID NO: 13 - DNA, 219 bp, synthetic construct
  misc_feature 1..219: note = Met-CCL17 cDNA
  source 1..219: mol_type = other DNA
atggctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttga

SEQ ID NO: 14 - protein, 71 aa, synthetic construct
  REGION 1..71: note = Met-CCL20
MASNFDCCLGYTDRILHPKFIVGFTRQLANEGCDINAIIFHTKKKLSVCANPKQTWVKYIVRLLSKKVKNM

SEQ ID NO: 15 - DNA, 216 bp, synthetic construct
  misc_feature 1..216: note = Met-CCL20 cDNA
  source 1..216: mol_type = other DNA
atggcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaa

SEQ ID NO: 16 - DNA, 207 bp, synthetic construct
  misc_feature 1..207: note = VTL101
  source 1..207: mol_type = other DNA
gtcgaaatgtccattcggctttttattggttttttttatacggcatatattggtatggctatcggatttatatgtagttcccccgatgcggagctgttttccgaaaaatcacgtatttcgtcttctgtcttgttaggatgtttgttgtgttgcatggattggtccgctgccgtacccgtctggtttggagcagggctcgatgtgtga

SEQ ID NO: 17 - DNA, 207 bp, synthetic construct
  misc_feature 1..207: note = VTL1 variant VTL1016
  source 1..207: mol_type = other DNA
gtcgaaatgtccattcggctttttattggtttcttttatacggcatatattggtatggctatcggatttatatgtagttcccccgatgcggagctgttttccgaaaaatcacgtatttcgtcttctgtcttgttaggatgtttgttgtgttgcatggattggtccgctgccgtacccgtctggtttggagcagggctcgatgtgtga

SEQ ID NO: 18 - DNA, 207 bp, synthetic construct
  misc_feature 1..207: note = VTL1 variant VTL1017
  source 1..207: mol_type = other DNA
gtcgaaatgtccatccgccttttcattggcttcttttacacagcatacatcgggatggctataggcttcatttgctcctctccagacgcggagctgttttcagagaaaagccggatatctagtagcgtgctgctcggatgtctgctctgttgcatggactggtccgctgccgtcccagtgtggttcggcgctggactggatgtgtga

SEQ ID NO: 19 - protein, 66 aa, Homo sapiens
MSIRLFIGFFYTAYIGMAIGFICSSPDAELFSEKSRISSSVLLGCLLCCMDWSAAVPVWFGAGLDV

SEQ ID NO: 20 - protein, 46 aa, synthetic construct
  REGION 1..46: note = cDNA encoding mature VTL1 protein
FICSSPDAELFSEKSRISSSVLLGCLLCCMDWSAAVPVWFGAGLDV

SEQ ID NO: 21 - DNA, 348 bp, synthetic construct
  misc_feature 1..348: note = VTL1 variant VTL1018
  source 1..348: mol_type = other DNA
gtcgaaatgtccattcggctttttattggtttcttttatacggcatatattggtatggctatcggatttatatgtagttcccccgatgcggagctgttttccgaaaaatcacgtatttcgtcttctgtcttgttaggatgtttgttgtgttgcatggattggtccgctgccgtacctgggaaaacagagccttttagaaaactttttgatgcaatcatgattaaaaagctaaaaagttgttctgctgcttacccgtctggtttggagcagggctcgatgtgtgatatggcagatgcatcgccgacaagtcttgaattaggattgtcgaaattagacaaagaatcatga

SEQ ID NO: 22 - DNA, 348 bp, synthetic construct
  misc_feature 1..348: note = VTL1 variant VTL1019
  source 1..348: mol_type = other DNA
gtcgaaatgtccattcggctttttattggttttttttatacggcatatattggtatggctatcggatttatatgtagttcccccgatgcggagctgttttccgaaaaatcacgtatttcgtcttctgtcttgttaggatgtttgttgtgttgcatggattggtccgctgccgtgccagggaaaacagagccttttagaaaactttttgatgcaatcatgattaaaaagctaaaaagttgttctgctgcttatccatctggtttggagcagggctcgatgtgtgatatggcagatgcatcgccgacaagtcttgaattaggattgtcgaaattagacaaagaatcatga

SEQ ID NO: 23 - DNA, 348 bp, synthetic construct
  misc_feature 1..348: note = VTL1 variant VTL1020
  source 1..348: mol_type = other DNA
gtcgaaatgtccattcggctttttattggtttcttttatacggcatatattggtatggctatcggatttatatgtagttcccccgatgcggagctgttttccgaaaaatcacgtatttcgtcttctgtcttgttaggatgtttgttgtgttgcatggattggtccgctgccgtgccagggaaaacagagccttttagaaaactttttgatgcaatcatgattaaaaagctaaaaagttgttctgctgcttatccatctggtttggagcagggctcgatgtgtgatatggcagatgcatcgccgacaagtcttgaattaggattgtcgaaattagacaaagaatcatga

SEQ ID NO: 24 - DNA, 348 bp, synthetic construct
  misc_feature 1..348: note = VTL1 variant VTL1021
  source 1..348: mol_type = other DNA
gtcgaaatgagcatcagactgttcatcggcttcttctacaccgcctacatcggcatggccatcggcttcatctgcagcagccccgacgccgagctgttcagcgagaagagcagaatcagcagcagcgtgctgctgggctgcctgctgtgctgcatggactggagcgccgccgtgccaggcaagaccgagcccttcagaaagctgttcgacgccatcatgatcaagaagctgaagagctgcagcgccgcctatcctagcggcctggagcagggcagcatgtgcgacatggccgacgccagccccaccagcctggagctgggcctgagcaagctggacaaggagagctga

SEQ ID NO: 25 - protein, 113 aa, synthetic construct
  REGION 1..113: note = VTL1 agonist protein
MSIRLFIGFFYTAYIGMAIGFICSSPDAELFSEKSRISSSVLLGCLLCCMDWSAAVPGKTEPFRKLFDAIMIKKLKSCSAAYPSGLEQGSMCDMADASPTSLELGLSKLDKES

SEQ ID NO: 26 - protein, 93 aa, synthetic construct
  REGION 1..93: note = VTL1 agonist mature protein
FICSSPDAELFSEKSRISSSVLLGCLLCCMDWSAAVPGKTEPFRKLFDAIMIKKLKSCSAAYPSGLEQGSMCDMADASPTSLELGLSKLDKES

SEQ ID NO: 27 - DNA, 276 bp, Homo sapiens
  source 1..276: mol_type = other DNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 28 - DNA, 285 bp, Homo sapiens
  source 1..285: mol_type = other DNA
atggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttga

SEQ ID NO: 29 - DNA, 291 bp, Homo sapiens
  source 1..291: mol_type = other DNA
atgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaa